(19) United States
(12) Patent Application Publication
(10) Pub. No.: US 2023/0234928 A1
(43) Pub. Date: Jul. 27, 2023

(54) Novel Compounds for Treating Fibrosis and Inflammatory Conditions

(71) Applicant: MAKScientific, LLC, Burlington, MA (US)

(72) Inventors: Alexandros Makriyannis, Watertown, MA (US); Kiran Vemuri, Boston, MA (US)

(21) Appl. No.: 18/007,199

(22) PCT Filed: Jul. 27, 2021
(86) PCT No.: PCT/US2021/043332

§ 371 Date: Jan. 27, 2023

Related U.S. Application Data
(60) Provisional application No. 63/056,892, filed on Jul. 27, 2020.

(51) Int. Cl. C07D 233/90; A61P 3/10

(52) U.S. Cl. CPC C07D 233/90; A61P 3/10; C07B 2200/05

(57) ABSTRACT

Several biological targets have been implicated in the pathogenesis of lung, liver, renal and prostrate fibrotic proliferative diseases. While cannabinoid receptor-mediated signaling has emerged as a novel signaling pathway regulating fibrogenesis and inflammation, the present invention relates generally to biologically active compounds capable of interacting with one or more biological targets including the CB1 and/or the CB2 cannabinoid receptors, and comprise of neutral antagonists, neutral-peripheral antagonists, peripheral antagonists/inverse-agonists, compounds with mixed properties including CB1 antagonist/CB2 agonist properties, and allosteric properties for treating fibrosis of the liver, lung, kidney and the prostrate, and inflammatory conditions, including benign prostatic hyperplasia. Also, aspects of the invention are concerned with imidazoles, triazoles, thiazoles, oxazoles, pyrazoles and dihydropyrazoles containing the 4-(λ2-azaneyl)thiomorpholine 1,1-dioxide group or the 4λ2-thiomorpholine 1,1-dioxide group.

### BACKGROUND

The present invention relates generally to biologically active compounds comprising of imidazoles, triazoles, thiazoles, oxazoles, pyrazoles and dihydropyrazoles capable of interacting with one or more biological targets including the CB1 and/or the CB2 cannabinoid receptors, and comprise of neutral antagonists, inverse-agonists, neutral-peripheral antagonists, peripheral antagonists/inverse-agonists, compounds with mixed properties including CB1 antagonist/CB2 agonist properties, and allosteric properties for treating fibrosis of the liver, lung, kidney and the prostrate, and inflammatory conditions, including benign prostatic hyperplasia. Another aspect of the invention is concerned with such compounds having a range of useful applications including use of certain neutral antagonists, inverse-agonists, compounds with mixed properties including CB1 antagonist/CB2 agonist properties, and allosteric properties to treat medical conditions with no or substantially reduced incidence of side-effects. Other aspects of the invention are concerned with new and improved compounds acting as neutral antagonists, inverse-agonists, compounds with mixed properties including CB1 antagonist/CB2 agonist properties, and allosteric properties selective for the CB1 and/or the CB2 receptors and use of these new and improved analogs as peripherally acting or centrally acting compounds, preferentially. Also, aspects of the invention are concerned with imidazoles, triazoles, thiazoles, oxazoles, pyrazoles and dihydropyrazoles containing the 4-(λ²-azaneyl)thiomorpholine 1,1-dioxide group or the 4λ²-thiomorpholine 1,1-dioxide group. In other aspects, the invention relates to solvates, polymorphs and all isotopic variations of compounds disclosed in the invention, combination therapy and methods of administering therapeutically effective amounts of same to provide a physiological effect.

Marijuana (Cannabis sativa) and derivatives have been used for medicinal and recreational purposes. The major active constituent extracted from Cannabis sativa is the classical cannabinoid Δ⁹-tetrahydrocannabinol (Δ⁹-THC). The effects of such cannabinoids are due to an interaction with specific receptors in the brain and the periphery. Presently, two cannabinoid receptors have been characterized: CB1, a central receptor found in the mammalian brain and a number of other sites in peripheral tissues; and CB2, a peripheral receptor found principally in cells related to the immune system. The CB1 receptor is believed to mediate the psychoactive properties associated with classical cannabinoids. Characterization of both CB1 and CB2 receptors has been made possible by X-ray and cryoelectron microscopy techniques while utilizing specific compounds that have affinity for either CB1, CB2, or both. CB1 and CB2 compounds with unique pharmacological properties and therapeutic value have been disclosed in WO200674445, WO2008154015 and WO2010104488.

### SUMMARY

Briefly stated, an embodiment of the invention is concerned with new and improved analogs that can act on the cannabinoid receptors. The inventive cannabimimetic imidazole ligands of this embodiment can be represented by general formula I and their enantiomers, diastereomers, geometric isomers, racemates, tautomers, rotamers, atropisomers, metabolites, in vivo hydrolysable esters, N-oxides, salts, solvates, hydrates, polymorphic forms (crystalline or amorphous) or pro-drugs:

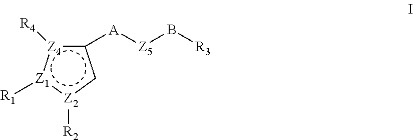

its solvates, polymorphs and isotopic variations thereof, wherein:
the ring system can contain 1 or 2 double bonds;
Z1, Z2, Z3 and Z4 comprise C, N, O or S;
A is N(R5) or a direct bond,

### B is N(R5),

R5 is hydrogen, OH, alkyl or substituted alkyl;

Z5 is optionally present and if present comprises —C(═O)—
R1 comprises -T-(CH₂)ₘ-Q-(CH₂)ₙ—Z,
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q comprises CH═CH, C≡C;
Z comprises
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₈ and X₁₀ each independently comprise H or alkyl
Wherein m is an integer from 0 to 7
j is an integer from 0 to about 6, or
k is an integer from 0 to about 2; or
R1 comprises -T-(CH₂)ₘ-Q-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; Q comprises CH═CH, C≡C; and

Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R1 comprises -T-(CH₂)ₘ-Q-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q comprises CH═CH, C≡C; and
Z comprises 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 comprises -T-(CH₂)ₘ-Q-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; Q comprises CH═CH, C≡C; and

Z comprises

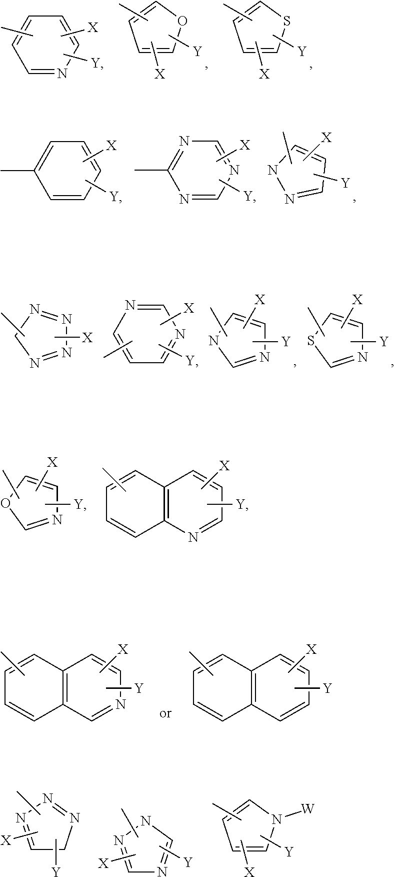

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₈ and X₁₀ each independently comprise H or alkyl
Wherein m is an integer from 0 to 7
j is an integer from 0 to about 6,
k is an integer from 0 to about 2,
W comprises H or alkyl; or
R1 comprises -T-(CH₂)ₘ-Q-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; Q comprises CH═CH, C≡C; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or

R1 comprises -T-(CH₂)ₘ-Q-(CH₂)ₙ—Z;

T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n independently comprises an integer from 0 to about 7;
Q comprises CH═CH, C≡C;
Z comprises

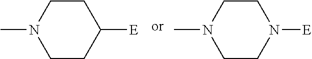

E comprises a C1 to about C4, linear or branched alkyl group, a phenyl group, a substituted phenyl group, a benzyl group or a substituted benzyl group; or

R1 comprises -T-(CH₂)ₘ-Q-(CH₂)ₙ—Z;

T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n independently comprises an integer from 0 to about 7;
Q comprises CH═CH, C≡C; and
Z comprises

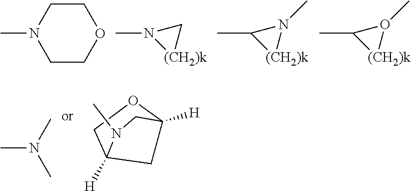

k is an integer from 1 to about 5, and
A₁ and A₂ each independently comprise a C1 to about C4 alkyl group, a phenyl group or a substituted phenyl group; or
R1 comprises —(CH₂)ₙ—Z;
n is an integer from 0 to about 7;
wherein Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₈ and X₁₀ each independently comprise H or alkyl
Wherein m is an integer from 0 to 7
j is an integer from 0 to about 6, or
k is an integer from 0 to about 2; or
R1 comprises —(CH₂)ₙ—Z;
n is an integer from 0 to about 7; and
Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 comprises —(CH₂)ₙ—Z;
n is an integer from 0 to about 7; and
Z comprises a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 comprises —(CH₂)ₙ—Z;
n is an integer from 0 to about 7; and
Z comprises 1-, 2- or 3-pyrrolidinyl, 1-, 2-, 3- or 4-piperidinyl, 2-, 3- or 4-morpholinyl, 2-3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R1 comprises —(CH₂)ₙ—Z;

n is an integer from 0 to about 7; and
Z comprises

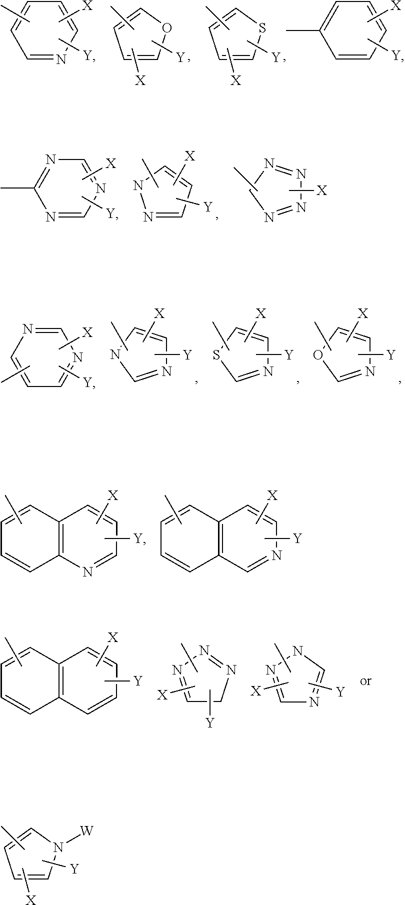

wherein X and Y each independently comprise, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2
W comprises H or alkyl; or
R1 comprises —(CH₂)ₙ—Z;
n is an integer from 0 to about 7; and
Z comprises a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R1 comprises —(CH₂)ₙ—Z;
n comprises an integer from 0 to about 7; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 comprises —(CH₂)ₘ-Q₁-(CH₂)ₙ—Z;
Q₁ comprises NH, O, S, —CH═CH—, —C≡C—, —CO, SO₂ or OSO₂;
m is an integer from 1 to about 7;
n is an integer from 0 to about 7; and
wherein Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2; or
R1 comprises -Q₂-(CH₂)ₙ—Z;
Q₂ is optionally present and if present comprises —CH₂—NH, —CH₂—O, —CH₂—S, —CH₂—SO₂ or —CH₂—OSO₂;
n is an integer from 0 to about 7;
wherein Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2; or
R1 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7;
Z comprises a bicyclic ring, a heterobicyclic ring, a tricyclic ring, a heterotricyclic ring, a polycyclic ring or a heteropolycyclic ring; or
R1 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7;

Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R1 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7;
Z comprises 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and

Z comprises

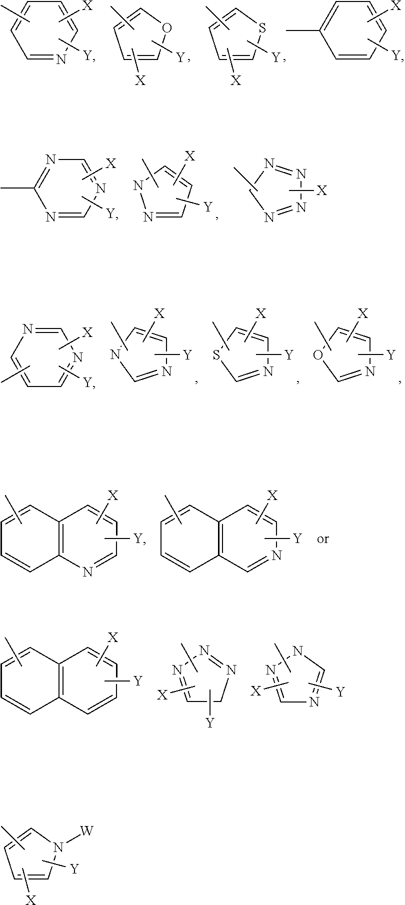

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2
W comprises H or alkyl; or
R1 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and

Z comprises

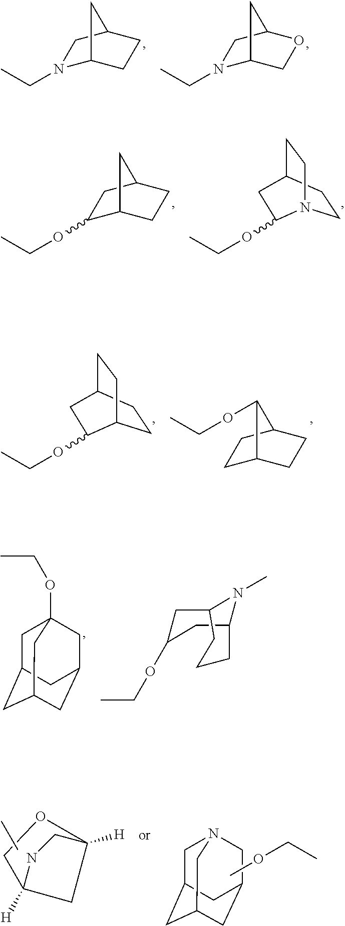

R1 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2; or
R1 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and

Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R1 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and

Z comprises 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R1 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and

Z comprises

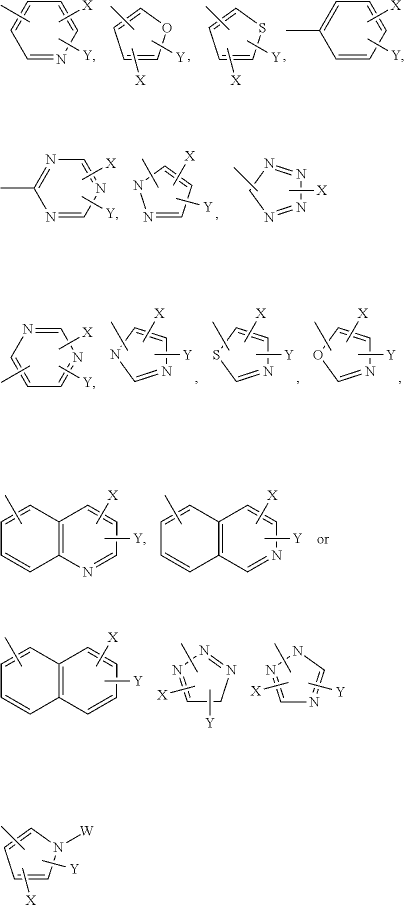

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, al, , NO₂, (CH₂)ₘCN, hydroxyloweralkyl, or alkyl-NX₁X₂,
X₄, X₈, and X each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₈ and X₁₀ each independently comprise H or alkyl
Wherein m is an integer from 0 to 7
j is an integer from 0 to about 6,
k is an integer from 0 to about 2
W comprises H or alkyl; or
R1 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 comprises -T-Q-(CH₂)ₙ—Z,
each n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q comprises CH═CH, C≡C;
Z comprises H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2; or
R1 comprises -T-Q-(CH₂)ₙ—Z;
each n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q comprises CH═CH, C≡C; and

Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R1 comprises -T-Q-(CH₂)ₙ—Z;
each n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q comprises CH═CH, C≡C; and

Z comprises 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R1 comprises -T-Q-(CH₂)ₙ—Z;
each n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q comprises CH═CH, C≡C; and

Z comprises

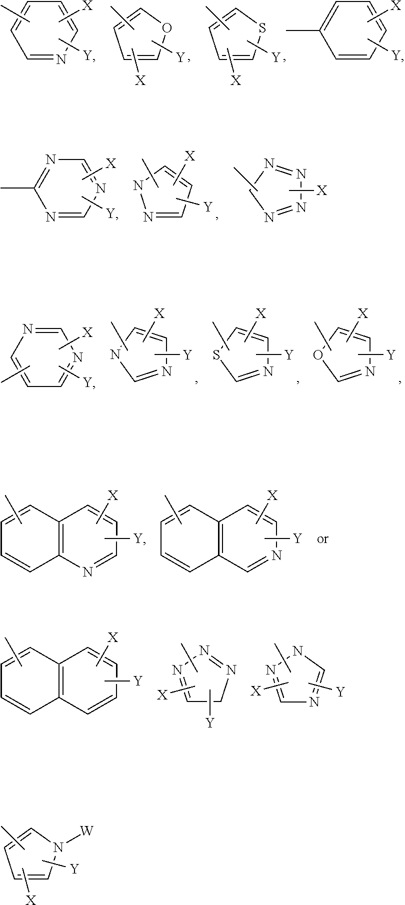

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2,
W comprises H or alkyl; or
R1 comprises -T-Q-(CH₂)ₙ—Z;
each n independently comprises an integer from 0 to about 7; T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q comprises CH═CH, C≡C; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or

R1 comprises -T-Q-(CH₂)ₙ—Z;

T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; each n independently comprises an integer from 0 to about 7;
Q comprises CH═CH, C≡C;
Z comprises

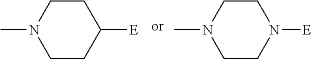

E comprises a C1 to about C4, linear or branched alkyl group, a phenyl group, a substituted phenyl group, a benzyl group or a substituted benzyl group; or

R1 comprises -T-Q-(CH₂)ₙ—Z;

T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; each n independently comprises an integer from 0 to about 7;
Q comprises CH═CH, C≡C; and
Z comprises

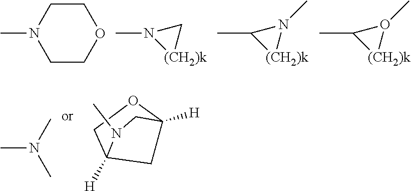

k is an integer from 1 to about 5, and
A₁ and A₂ each independently comprise a C1 to about C4 alkyl group, a phenyl group or a substituted phenyl group;
R2 comprises —(CH₂)ₙ—Z;
n is an integer from 0 to about 7;
wherein Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2; or
R2 comprises —(CH₂)ₙ—Z;
n is an integer from 0 to about 7; and
Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 comprises —(CH₂)ₙ—Z;
n is an integer from 0 to about 7; and
Z comprises a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 comprises —(CH₂)ₙ—Z;
n is an integer from 0 to about 7; and
Z comprises 1-, 2- or 3-pyrrolidinyl, 1-, 2-, 3- or 4-piperidinyl, 2-, 3- or 4-morpholinyl, 2-3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R2 comprises —(CH₂)ₙ—Z;

n is an integer from 0 to about 7; and
Z comprises

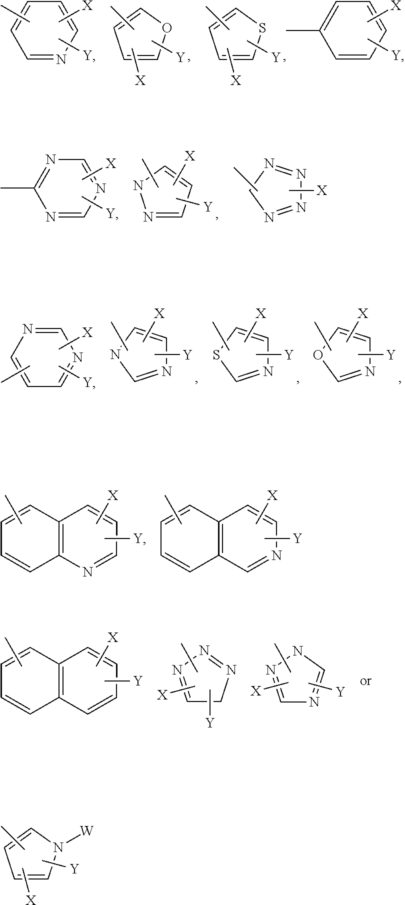

wherein X and Y each independently comprise, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2
W comprises H or alkyl; or
R2 comprises —(CH₂)ₙ—Z;
n is an integer from 0 to about 7; and
Z comprises a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R2 comprises —(CH₂)ₙ—Z;
n comprises an integer from 0 to about 7; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 comprises —(CH₂)ₘ-Q₁-(CH₂)ₙ—Z;
Q₁ comprises NH, O, S, —CH═CH—, —C≡C—, —CO, SO₂ or OSO₂;
m is an integer from 1 to about 7;
n is an integer from 0 to about 7; and
wherein Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2; or
R2 comprises -Q₂-(CH₂)ₙ—Z;
Q₂ is optionally present and if present comprises —CH₂—NH, —CH₂—O, —CH₂—S, —CH₂—SO₂ or —CH₂—OSO₂;
n is an integer from 0 to about 7;
wherein Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2; or
R2 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7;

Z comprises a bicyclic ring, a heterobicyclic ring, a tricyclic ring, a heterotricyclic ring, a polycyclic ring or a heteropolycyclic ring; or

R2 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7;

Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R2 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7;

Z comprises 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R2 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and

Z comprises

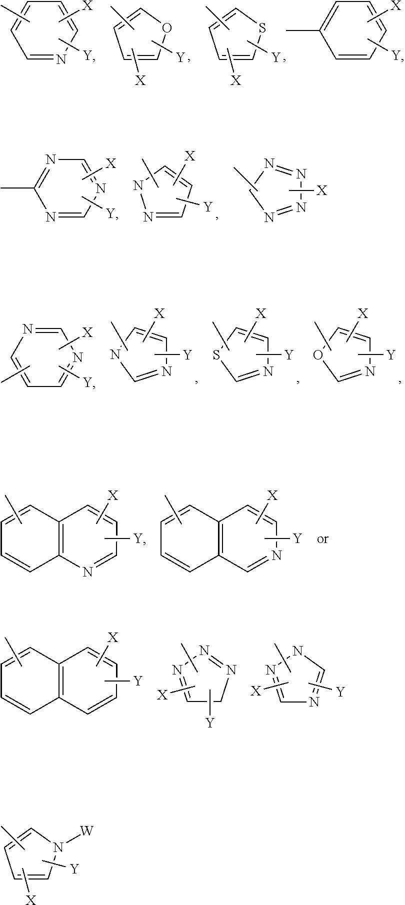

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2
W comprises H or alkyl; or
R2 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and

Z comprises

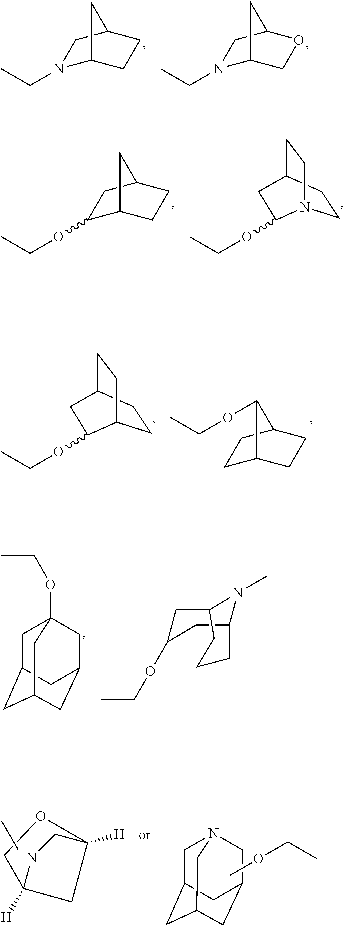

R2 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2; or
R2 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and

Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R2 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and

Z comprises 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R2 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and

Z comprises

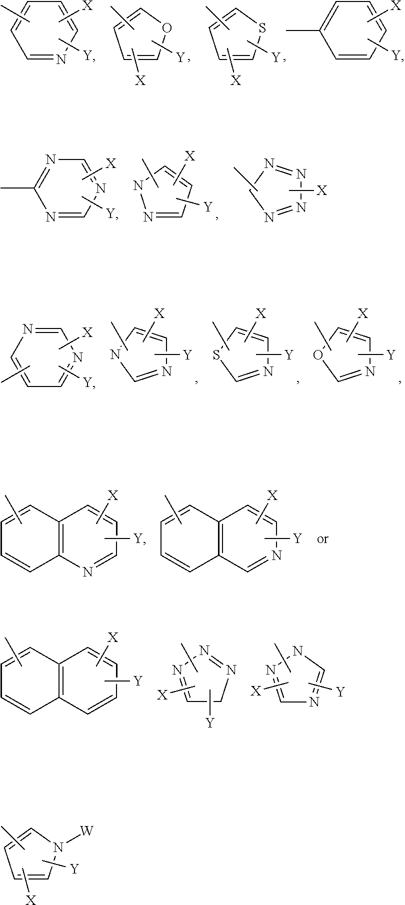

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈,
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₈, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2
W comprises H or alkyl; or
R2 comprises -T-(CH₂)ₙ—Z;
n comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R2 comprises -T-(CH₂)ₘ-Q₁-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ comprises NH, O, S, CH═CH, C≡C, CO, SO₂ or OSO₂; and
Z comprises, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2; or
R2 comprises -T-(CH₂)ₘ-Q₁-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ comprises NH, O, S, CH═CH, C≡C, CO, SO₂ or OSO₂; and

Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R2 comprises -T-(CH₂)ₘ-Q₁-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ comprises NH, O, S, CH═CH, C≡C, CO, SO₂ or OSO₂; and

Z comprises 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or

R2 comprises -T-(CH₂)ₘ-Q₁-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ comprises N, O, S, CH═CH, C≡C, CO, SO₂ or OSO₂; and

Z comprises

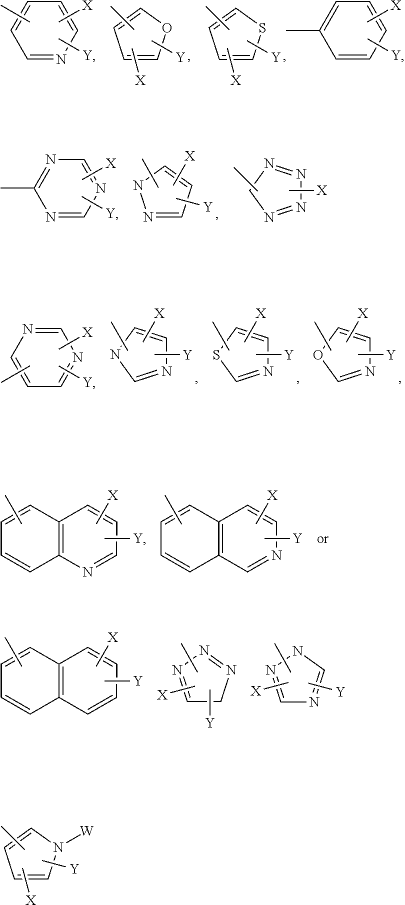

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, aryl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2
W comprises H or alkyl; or
R2 comprises -T-(CH₂)ₘ-Q₁-(CH₂)ₙ—Z;
m and n independently comprises an integer from 0 to about 7;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ comprises NH, O, S, CH═CH, C≡C, CO, SO₂ or OSO₂; and
Z comprises an unsaturated ring having 5 ring atoms and 0 to 2 independently selected heteroatoms as ring members fused to an unsaturated ring having 5 ring atoms and 0 to 2 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members, or

R2 comprises -T-(CH₂)ₘ-Q₁-(CH₂)ₙ—Z;

T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n independently comprises an integer from 0 to about 7;
Q₁ comprises NH, O, S, CH═CH, C≡C, CO, SO₂ or OSO₂; and
Z comprises:

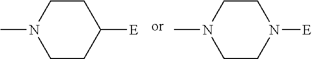

E comprises a C1 to about C4, linear or branched alkyl group, a phenyl group, a substituted phenyl group, a benzyl group or a substituted benzyl group; or
R2 comprises -T-(CH₂)ₘ-Q₁-(CH₂)ₙ—Z;
T comprises a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n independently comprises an integer from 0 to about 7;
Q₁ comprises NH, O, S, CH═CH, C≡C, CO, SO₂ or OSO₂;
Z comprises

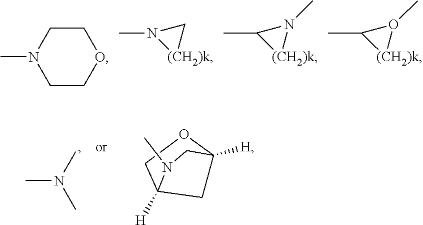

k is an integer from 1 to about 5,
A₁ and A₂ each independently comprise a C1 to about C4 alkyl group, a phenyl group or a substituted phenyl group;

R3 comprises

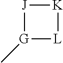

wherein G comprises CH, C(CH₃), C(CN) or N,
L, K and J each independently comprise (CH₂)ₙ, (CH₃)₂, C═O, O, —CHOH, C(CH₃)OM₁, C(CH₂)ₙ(X)Y, NM₁, SO₂ SO or S,
and at least one of L, K or J is SO₂,
n is an integer from 0 to about 7;
M₁ is H, alkyl, C(O)M₂ where
M₂ is H, alkyl, NM₃M₄, OM₅ and M₃, M₄ and M₅ are independently H, OH or alkyl and X and Y each independently comprise, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2;
R4 comprises
H, halogen, CF₃, CF₂H, N₃, NCS, CN, NO₂, NX₁X₂, OH, ONO₂, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2; or

R4 comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring or a heteropolycyclic ring; or

R4 comprises

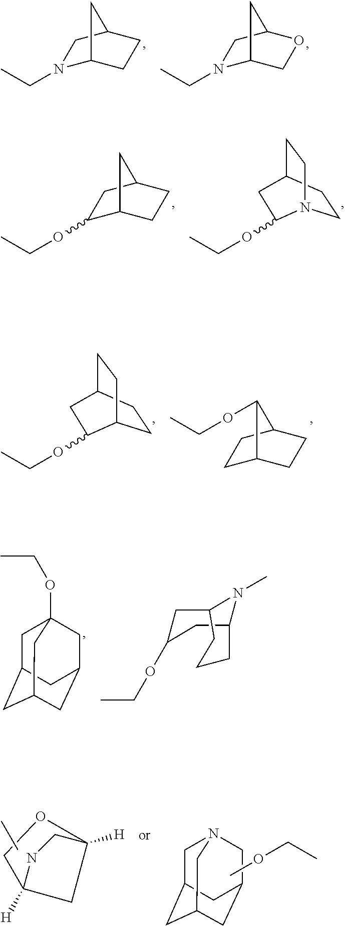

R4 comprises —(CH₂)_{d}—Z;
d is an integer from 1 to about 6;
Z comprises H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2; or
R4 comprises —(CH₂)_{d}—Z;
d is an integer from 1 to about 6; and
Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 comprises —(CH₂)_{d}—Z;
d is an integer from 1 to about 6; and
Z comprises 1-, 2- or 3-pyrrolidinyl, 1-, 2-, 3- or 4-piperidinyl, 2-, 3- or 4-morpholinyl, 2-3- or 4-thiomorpholinyl, 1-, 2- or 3-azetidinyl, 1- or 2-piperazinyl, 2- or 3-tetrahydrofuranyl; or any above group substituted on at least one ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6, or
k is an integer from 0 to about 2; or
R4 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises 1-, 2- or 3-pyrrolidinyl, 1-, 2-, 3- or 4-piperidinyl, 2-, 3- or 4-morpholinyl, 2-3- or 4-thiomorpholinyl, 1-, 2- or 3-azetidinyl, 1- or 2-piperazinyl, 2- or 3-tetrahydrofuranyl; or any above group substituted on at least one available ring atom by an alkyl; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 comprises —(CH₂)ₘ-Q₁-(CH₂)ₙ—Z;
Q₁ comprises NH, O, S, CH═CH, C≡C, CO, SO₂ or OSO₂;
m is an integer from 1 to about 7;
n is an integer from 0 to about 7; and
Z comprises

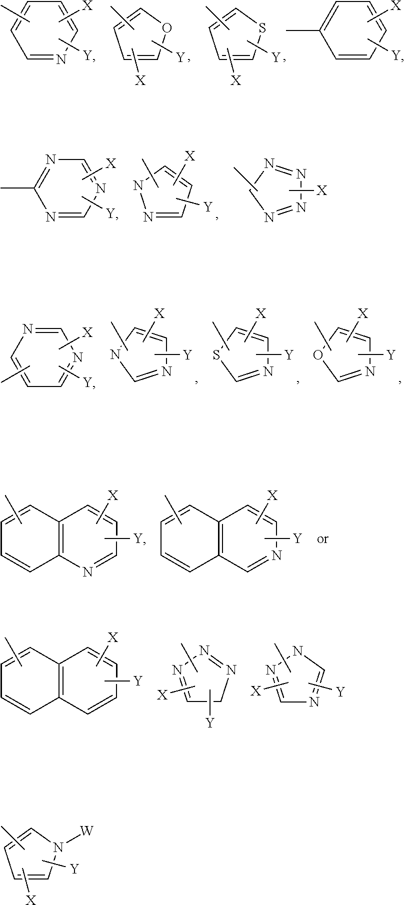

wherein X and Y each independently comprise
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ each independently comprise H or alkyl, or
X₁ and X₂ together comprise part of a heterocyclic ring having about 4 to about 7 ring members and optionally one additional heteroatom selected from O, N or S, or
X₁ and X₂ together comprise part of an imide ring having about 5 to about 6 members,
X₃ comprises H, alkyl, NO₂, (CH₂)ₘCN, hydroxyloweralkyl, or alkyl-NX₁X₂,
X₄, X₅, and X₆ each independently comprise H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ comprises H, alkyl, carbocyclic ring, heterocyclic ring, aromatic ring, heteroaromatic ring, or —CX₉═CHX₁₀ wherein
X₉ and X₁₀ each independently comprise H or alkyl
Wherein m is an integer from 0 to 7
j is an integer from 0 to about 6,
k is an integer from 0 to about 2,
W comprises H or alkyl.

A variation of formula I comprises cannabimimetic imidazole ligands represented by formula II and their enantiomers, diastereomers, geometric isomers, racemates, tautomers, rotamers, atropisomers, metabolites, in vivo hydrolysable esters, N-oxides, salts, solvates, hydrates, polymorphic forms (crystalline or amorphous), pro-drugs drugs and all isotopic variations:

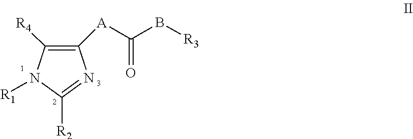

A variation of formula I comprises cannabimimetic thiazole and oxazole ligands represented by formula III and their enantiomers, diastereomers, geometric isomers, racemates, tautomers, rotamers, atropisomers, metabolites, in vivo hydrolysable esters, N-oxides, salts, solvates, hydrates, polymorphic forms (crystalline or amorphous), pro-drugs drugs and all isotopic variations:

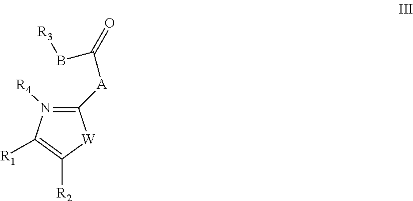

A variation of formula I comprises cannabimimetic triazole ligands represented by formula IV and their enantiomers, diastereomers, geometric isomers, racemates, tautomers, rotamers, atropisomers, metabolites, in vivo hydrolysable esters, N-oxides, salts, solvates, hydrates, polymorphic forms (crystalline or amorphous), pro-drugs drugs and all isotopic variations:

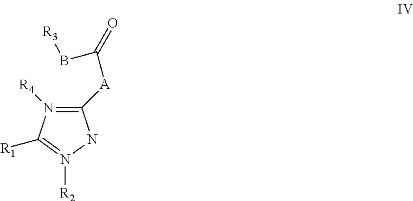

A variation of formula I comprises cannabimimetic pyrazoline or chiral dihydropyrazole ligands represented by formula V and their enantiomers, diastereomers, geometric isomers, racemates, tautomers, rotamers, atropisomers, metabolites, in vivo hydrolysable esters, N-oxides, salts, solvates, hydrates, polymorphic forms (crystalline or amorphous), pro-drugs drugs and all isotopic variations:

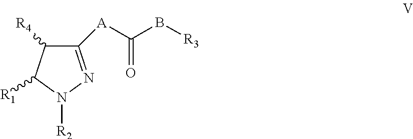

A variation of formula I comprises cannabimimetic pyrazoline or chiral dihydropyrazole ligands represented by formula VI and their enantiomers, diastereomers, geometric isomers, racemates, tautomers, rotamers, atropisomers, metabolites, in vivo hydrolysable esters, N-oxides, salts, solvates, hydrates, polymorphic forms (crystalline or amorphous), pro-drugs drugs and all isotopic variations:

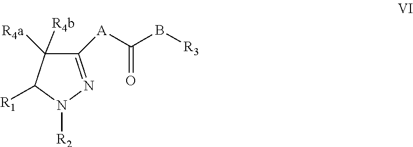

wherein R4a and R4b are each independently H, alkyl or halogen.

A variation of formula I comprises cannabimimetic pyrazoline or chiral dihydropyrazole ligands represented by formula VII and their enantiomers, diastereomers, geometric isomers, racemates, tautomers, rotamers, atropisomers, metabolites, in vivo hydrolysable esters, N-oxides, salts, solvates, hydrates, polymorphic forms (crystalline or amorphous), pro-drugs drugs and all isotopic variations:

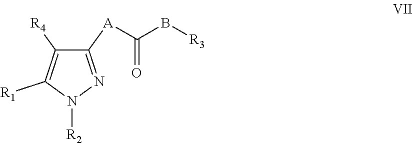

The inventive compounds in any formula, embodiment or variation include any and all possible isomers and steroisomers. In general, the compositions of the invention may be alternately formulated to comprise, consist of, or consist essentially of, any appropriate components herein disclosed. The compositions of the invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

Unless otherwise specifically defined, “acyl” refers to the general formula —C(O)alkyl.

Unless otherwise specifically defined, “acyloxy” refers to the general formula —O-acyl.

Unless otherwise specifically defined, “alcohol” refers to the general formula alkyl-OH and includes primary, secondary and tertiary variations.

Unless otherwise specifically defined, “alkyl” or “lower alkyl” refers to a linear, branched or cyclic alkyl group having from 1 to about 10 carbon atoms, and advantageously 1 to about 7 carbon atoms including, for example, methyl, ethyl, propyl, butyl, hexyl, octyl, isopropyl, isobutyl, tert-butyl, cyclopropyl, cyclohexyl, cyclooctyl, vinyl and allyl. The alkyl group can be saturated or unsaturated. The alkyl group can be unsubstituted, singly substituted or, if possible, multiply substituted, with substituent groups in any possible position. Unless otherwise specifically limited, a cyclic alkyl group includes monocyclic, bicyclic, tricyclic, tetracyclic and polycyclic rings, for example norbornyl, adamantyl and related terpenes.

Unless otherwise specifically defined, “alkoxy” refers to the general formula —O-alkyl.

Unless otherwise specifically defined, “alkylmercapto” refers to the general formula —S-alkyl.

Unless otherwise specifically defined, “alkylamino” refers to the general formula —(NH)-alkyl.

Unless otherwise specifically defined, “di-alkylamino” refers to the general formula —N-(alkyl)₂. Unless otherwise specifically limited di-alkylamino includes cyclic amine compounds such as piperidine and morpholine.

Unless otherwise specifically defined, an aromatic ring is an unsaturated ring structure having about 5 to about 7 ring members and including only carbon as ring atoms. The aromatic ring structure can be unsubstituted, singly substituted or, if possible, multiply substituted, with substituent groups in any possible position.

Unless otherwise specifically defined, “aryl” refers to an aromatic ring system that includes only carbon as ring atoms, for example phenyl, biphenyl or naphthyl. The aryl group can be unsubstituted, singly substituted or, if possible, multiply substituted, with substituent groups in any possible position.

Unless otherwise specifically defined, “aroyl” refers to the general formula —C(═O)-aryl.

Unless otherwise specifically defined, a bicyclic ring structure comprises 2 fused or bridged rings that include only carbon as ring atoms. The bicyclic ring structure can be saturated or unsaturated. The bicyclic ring structure can be unsubstituted, singly substituted or, if possible, multiply substituted, with substituent groups in any possible position. The individual rings may or may not be of the same type. Examples of bicyclic ring structures include naphthalene and bicyclooctane.

Unless otherwise specifically defined, a carbocyclic ring is a non-aromatic ring structure, saturated or unsaturated, having about 3 to about 8 ring members that includes only carbon as ring atoms, for example, cyclohexadiene or cyclohexane. The carbocyclic ring can be unsubstituted, singly substituted or, if possible, multiply substituted, with substituent groups in any possible position.

Unless otherwise specifically defined, “halogen” refers to an atom selected from fluorine, chlorine, bromine and iodine.

Unless otherwise specifically defined, a heteroaromatic ring is an unsaturated ring structure having about 5 to about 8 ring members independently selected from carbon atoms and one or more heteroatoms, including oxygen, nitrogen and/or sulfur, for example, pyridine, furan, quinoline, and their derivatives. The heteroaromatic ring can be unsubstituted, singly substituted or, if possible, multiply substituted, with substituent groups in any possible position.

Unless otherwise specifically defined, a heterobicyclic ring structure comprises 2 fused or bridged rings having ring members independently selected from carbon and one or more heteroatoms, including oxygen, nitrogen and/or sulfur. The heterobicyclic ring structure can be saturated or unsaturated. The heterobicyclic ring can be unsubstituted, singly substituted or, if possible, multiply substituted, with substituent groups in any possible position. The individual rings may or may not be of the same type. Examples of heterobicyclic ring structures include isobenzofuran and indole.

Unless otherwise specifically defined, a heterocyclic ring is a saturated or unsaturated ring structure having about 3 to about 8 ring members independently selected from carbon atoms and one or more heteroatoms, including oxygen, nitrogen and/or sulfur; for example, piperidine, morpholine, piperazine, pyrrolidine, thiomorpholine, 1,1-dioxothiomorpholine and their derivatives. The heterocyclic ring can be unsubstituted, singly substituted or, if possible, multiply substituted, with substituent groups in any possible position.

Unless otherwise specifically defined, a heterotricyclic ring structure comprises 3 fused, bridged, or both fused and bridged rings having ring members independently selected from carbon and one or more heteroatoms, including oxygen, nitrogen and/or sulfur. The heterotricyclic ring structure may be saturated or unsaturated. The heterotricyclic ring structure can be unsubstituted, singly substituted or, if possible, multiply substituted, with substituent groups in any possible position. The individual rings may or may not be of the same type. Examples of heterotricyclic ring structures include carbazole, phenanthroline, phenazine, 2,4,10-trioxaadamantane and tetradecahydro-phenanthroline.

Unless otherwise specifically defined, a heteropolycyclic ring structure comprises more than 3 rings that may be fused, bridged or both fused and bridged and that have ring members independently selected from carbon and one or more heteroatoms, including oxygen, nitrogen and/or sulfur. The heteropolycyclic ring structure can be saturated or unsaturated. The heteropolycyclic ring structure can be unsubstituted, singly substituted or, if possible, multiply substituted, with substituent groups in any possible position. The individual rings may or may not be of the same type. Examples of heteropolycyclic ring structures include azaadamantine, tropane, homotropane and 5-norbornene-2,3-dicarboximide.

Unless otherwise specifically defined, the term “phenacyl” refers to the general formula -phenyl-acyl.

Unless otherwise specifically defined, a polycyclic ring structure comprises more than 3 rings that may be fused, bridged or both fused and bridged, and that includes carbon as ring atoms. The polycyclic ring structure can be saturated or unsaturated. The polycyclic ring structure can be unsubstituted, singly substituted or, if possible, multiply substituted, with substituent groups in any possible position. The individual rings may or may not be of the same type. Examples of polycyclic ring structures include adamantine, bicyclooctane, norbornane and bicyclononanes.

Unless otherwise specifically defined, a spirocycle refers to a ring system wherein a single atom is the only common member of two rings. A spirocycle can comprise a saturated carbocyclic ring comprising about 3 to about 8 ring members, a heterocyclic ring comprising about 3 to about 8 ring atoms wherein up to about 3 ring atoms may be N, S, or O or a combination thereof.

Unless otherwise specifically defined, a tricyclic ring structure comprises 3 rings that may be fused, bridged or both fused and bridged, and that includes carbon as ring atoms. The tricyclic ring structure can be saturated or unsaturated. The tricyclic ring structure can be unsubstituted, singly substituted or, if possible, multiply substituted, with substituent groups in any possible position. and may be substituted or unsubstituted. The individual rings may or may not be of the same type. Examples of tricyclic ring structures include fluorene and anthracene.

Unless otherwise specifically limited the term substituted means substituted by a below-described substituent group in any possible position. Substituent groups for the above moieties useful in the invention are those groups that do not significantly diminish the biological activity of the inventive compound. Substituent groups that do not significantly diminish the biological activity of the inventive compound include, for example, H, halogen, N₃, NCS, CN, NO₂, NX₁X₂, OX₃, C(X₃)₃, OAc, O-acyl, O-aroyl, NH-acyl, NH-aroyl, NHCOalkyl, CHO, C(halogen)₃, COOX₃, SO₃H, PO₃H₂, SO₂NX₁X₂, CONX₁X₂, alkyl, alcohol, alkoxy, alkylmercapto, alkylamino, di-alkylamino, sulfonamide, thioalkoxy or methylene dioxy when the substituted structure has two adjacent carbon atoms, wherein X₁ and X₂ each independently comprise H or alkyl, or X₁ and X₂ together comprise part of a heterocyclic ring having about 4 to about 7 ring members and optionally one additional heteroatom selected from O, N or S, or X₁ and X₂ together comprise part of an imide ring having about 5 to about 6 members and X₃ comprises H, alkyl, hydroxyloweralkyl, or alkyl-NX₁X₂. Unless otherwise specifically limited a substituent group may be in any possible position.

Some of the inventive compounds show a high affinity for at least one of the cannabinoid receptors. Thus, another aspect of the invention is use of at least one of the inventive compounds to interact with a cannabinoid receptor.

Some of the novel heteropyrrole derivatives show selectivity for the CB1 cannabinoid receptor. These inventive CB1 selective analogs are able to interact with the CB1 cannabinoid receptors present in the CNS as well as the periphery without affecting the CB2 receptors to the same degree. Therefore, still another aspect of the invention is use of at least one of the inventive compounds to preferentially interact with a CB1 cannabinoid receptors present either in the CNS or the periphery.

Another aspect of the invention is use of at least one of the inventive compounds to interact with both CB1 and CB2 cannabinoid receptors present either in the CNS or the periphery.

The inventive heteropyrrole analogs described herein, and physiologically acceptable salts thereof, have pharmacological properties when administered in therapeutically effective amounts for providing a physiological response. Thus, another aspect of the invention is the administration of a therapeutically effective amount of at least one of the inventive compounds, or a physiologically acceptable salt thereof, to an individual or animal to provide a physiological response.

The subject matter of this invention also discloses novel processes and methods to synthesize compounds.

A better understanding of the invention will be obtained from the following detailed description of the article and the desired features, properties, characteristics, and the relation of the elements as well as the process steps, one with respect to each of the others, as set forth and exemplified in the description, illustrative embodiments and examples.

### BRIEF DESCRIPTION OF THE DRAWINGS

FIG. 1 is a graph showing competition binding data with hCB1-HEK292 cells (Compound 1);

FIG. 2 is a graph showing in vivo CB1 antagonism (hypothermia) data in male CD1 mice (Compound 1);

FIG. 3A-B show the effects of Compound 1 and rimonabant treatment on lung fibrosis development in a Non-alcoholic steatohepatitis (NASH) mouse model (anti-NASH/fibrotic activity);

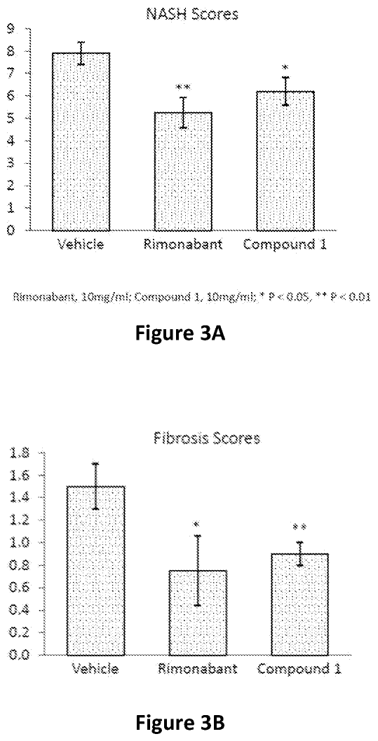

FIG. 4A-B show the effects of Compound 1 in reversing fibrosis in a diabetic high-fat diet mouse Non-alcoholic steatohepatitis (NASH) model; and

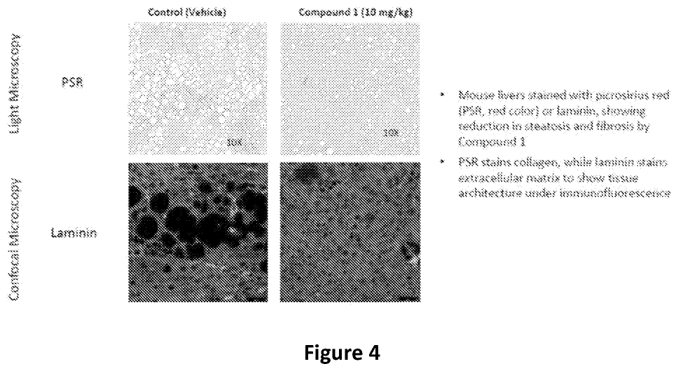

FIG. 5 shows the effects of Compound 1 and rimonabant treatment on survival in a Non-alcoholic steatohepatitis (NASH) mouse model.

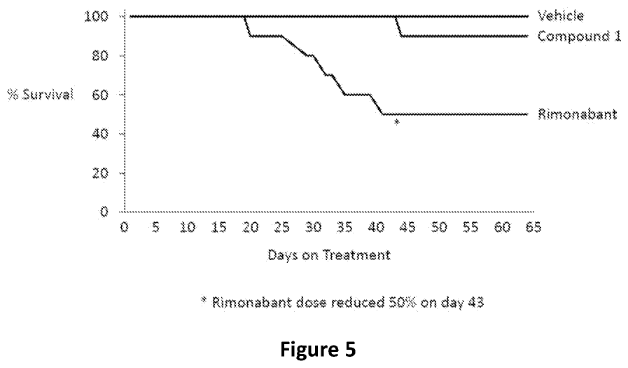

### DETAILED DESCRIPTION

As used herein a “therapeutically effective amount” of a compound, is the quantity of a compound which, when administered to an individual or animal, results in a sufficiently high level of that compound in the individual or animal to cause a physiological response, for example a discernible increase or decrease in stimulation of cannabinoid receptors. The inventive compounds described herein, and physiologically acceptable salts thereof, have pharmacological properties when administered in therapeutically effective amounts individually or in combination for providing a physiological response useful to treat fibrosis of the liver, lung or kidney, prostatic fibrosis and related inflammatory conditions including lower urinary tract symptoms, and benign prostatic hyperplasia.

Typically, a “therapeutically effective amount” of an inventive compound is believed to range from about 0.01 mg/day to about 1,000 mg/day.

As used herein, an “individual” refers to a human. An “animal” refers to, for example nonhuman-primates such as squirrel monkeys, rhesus monkeys, marmosets, baboons, veterinary animals, such as rodents, dogs, cats, horses and the like, and farm animals, such as cows, pigs and the like.

In certain embodiments, the compound disclosed in the invention can be used in combination with other acceptable pharmaceutical substances.

In embodiments in which compounds of the disclosure is used in combination with rimonabant (Accomplia, Sanofi-Aventis) or other CB1 antagonists, it will be possible to reduce or even eliminate one or more of these side-effects, particularly nausea. That is, it is possible to reduce the amount of Rimonabant or other CB1 antagonists administered to the individual who has had, is receiving or is about to receive a therapeutically effective amount of the compound of the disclosure. In one embodiment, the amount of rimonabant administered to the individual is reduced by 1.5 to 5-fold compared to the accepted therapeutic amount. The individual is then dosed with a therapeutically effective amount of at least one of the compounds of the disclosure. Of course, it is also possible to increase the length of time between doses of Rimonabant with the same or similar effect.

Accordingly, one embodiment provides for a method for reducing unwanted side-effects (one or more of nausea, dizziness, diarrhea, and anxiety) typically associated with administration of SR141716A (Accomplia™/Rimonabant) or other CB1 antagonists to certain individuals. A particular method involves administering a therapeutically effective amount of at least one of the compounds of the disclosure so as to reduce the side-effects in that individual. As discussed, the method can involve reducing the amount of SR141716A (Accomplia™/Rimonabant) or other CB1 antagonists administered to the individual.

As will be apparent, the compounds of the invention can be used alone or in combination with other CB1 receptor antagonists known to the field. Examples of such agents include SR141716A (Acomplia®/Rimonabant, 5-(4-chlorophenyl)-1-(2,4-dichlorophenyl)-4-methyl-N-(piperidin-1-yl)-1H-pyrazole-3-carboxamide), Xenical® (Orlistat, (S)-(S)-1-((2S,3S)-3-hexyl-4-oxooxetan-2-yl)tridecan-2-yl 2-formamido-4-methylpentanoate), Meridia® (Sibutramine, 1-(1-(4-chlorophenyl)cyclobutyl)-N,N,3-trimethylbutan-1-amine, hydrochloride monohydrate), SR147778 (Surinabant, 5-(4-bromophenyl)-1-(2,4-dichlorophenyl)-4-ethyl-N-(piperidin-1-yl)-1H-pyrazole-3-carboxamide), AVE-1625 (Drinabant, N-[1-[bis(4-chlorophenyl)methyl]-3-azetidinyl]-N-(3,5-difluorophenyl)-methanesulfonamide), CP-945,598 (Otenabant, 1-[8-(2-Chlorophenyl)-9-(4-chlorophenyl)-9H-purin-6-yl]-4-(ethylamino)piperidine-4-carboxamide), E-6776 (Rosonabant, 5-(4-chlorophenyl)-1-(2,4-dichlorophenyl)-N-(piperidin-1-yl)-4,5-dihydro-1H-pyrazole-3-carboxamide), MK-0364 (Taranabant, N-((2S,3S)-4-(4-chlorophenyl)-3-(3-cyanophenyl)butan-2-yl)-2-methyl-2-((5-(trifluoromethyl)pyridin-2-yl)oxy)propanamide), SLV-319 (Ibipinabant, (S,E)-3-(4-chlorophenyl)-N′-((4-chlorophenyl)sulfonyl)-N-methyl-4-phenyl-4,5-dihydro-1H-pyrazole-1-carboximidamide), V24343, Qsymia (Qnexa, Phentermine/topiramate, 2-methyl-1-phenylpropan-2-amine and 2,3:4,5-Bis-O-(1-methylethylidene)-p-D-fructopyranose sulfamate), Contrave (Bupropion/naltrexone, 2-(tert-butylamino)-1-(3-chlorophenyl)propan-1-one and 17-(cyclopropylmethyl)-4,5α-epoxy-3,14-dihydroxymorphinan-6-one), Empatic (Bupropion/zonisamide, 2-(tert-butylamino)-1-(3-chlorophenyl)propan-1-one and benzo[d]isoxazol-3-ylmethanesulfonamide), lorcaserin (Belviq, (1R)-8-chloro-1-methyl-2,3,4,5-tetrahydro-1H-3-benzazepine), and Phentermine (2-methyl-1-phenylpropan-2-amine).

Compounds of the invention can also be used in combination with a potassium channel opener, opioid antagonist, anticonvulsant agent, contraceptive agent, antipsychotic agent, anticonstipation agent, nicotine receptor agonist or partial agonist, CB2 agonist, melanin-concentrating hormone receptor antagonist, anti-psychotic agents, peroxisome proliferator-activated receptor modulator, inhibitors of the BET (bromodomain and extra terminal) domain protein, farnesoid X receptor agonists, ghrelin antagonists, GLP-1 agonist, fatty acid amide hydrolase inhibitor, an intestinal-acting microsomal triglyceride transfer protein inhibitor, a dipeptidyl-peptidase IV inhibitor, angiotensin-converting-enzyme inhibitor, a statin, a sterol absorption inhibitor (β-lactam), Beta-3 adrenergic agonist, a biguanide, Sodium glucose transport (SGLT2) antagonist, CCR2 and CCR5 receptor antagonists, cyclooxygenase-2 inhibitor, renin inhibitor, monoamine oxidase inhibitor, CETP inhibitor, ACAT inhibitor, DGAT-1 inhibitor, Mitochondrial Transfer Protein inhibitor, noradrenalin-serotonin-dopamine reuptake inhibitor or a lipase inhibitor.

In one embodiment, less than five compounds of the disclosure, preferably one or two of same is used in combination with less than five of the known CB1 antagonists, preferably one or two of same.

In one embodiment, the compound disclosed in the invention could in itself act as a drug with a combination effect. For example, compounds disclosed in the invention could dually act as a CB1 antagonist as well as 11β-hydroxy steroid dehydrogenase-1 inhibitor. In certain embodiments, the compound could act dually as a CB1 antagonist as well as a nitric oxide donor or a nitric oxide synthase inhibitor.

By “physiologically acceptable salts” is meant, salts typically useful for pharmaceutical applications including acid addition salts and basic salts. Examples of acid addition salts are hydrochloride salts, hydrobromide salts, methane sulfonate salts etc. Examples of basic salts are salts where the cation is selected from alkali metals, such as sodium and potassium, alkaline earth metals, such as calcium, and ammonium ions. Other examples of physiologically acceptable salts can be found in “Remington's Pharmaceutical Sciences” 17. Ed. Alfonso R. Gennaro (Ed.), Mark Publishing Company, Easton, Pa., U.S.A., 1985 and more recent editions, and in Encyclopedia of Pharmaceutical Technology.

Polymorphic forms show improved physiochemical properties and stability. In one embodiment, the compounds disclosed in the invention could exist in various solid forms. The solid forms can be crystalline and amorphous forms, but not limited to, solvates, hydrates, hydrolyzable esters and N-oxides of the compounds defined in the specification. These solid forms can be obtained by treating either the free base or their salts at a certain adjusted pH and certain temperature with an solvent or a combination of solvents. The solvents can be and not limited to a hydrocarbon solvent such as toluene, xylene, hexanes, heptane, or petroleum ether, alcohol such as methanol, ethanol, n-butanol, n-propanol and 2-propanol, di-isopropyl ether, ethyl-acetate, dichloromethane, acetic acid, acetone, tetrahydrofuran, dichloromethane, and water.

In certain embodiments, the application discloses a novel process of manufacturing compounds of formula I-VII wherein a prefinal step involving a purified carboxylate obtained from a purified acid derivative is reacted with hydrazine monohydrate and the final reaction involving the hydrazide derivative reacting with divinyl sulfone in the presence of a protic solvent.

In another embodiment the purified acid derivative is directly reacted with a substituted hydrazine such as 1-aminopiperidine, 4-aminomorpholine, 4-aminothiomorpholine 1,1-dioxide or 4-aminothiomorpholine 1,1-dioxide-2,2,3,3,5,5,6,6-d8 in the presence of a peptide coupling reagent such as N,N,N′,N′-tetramethyl-O-(benzotriazol-1-yl)uronium tetrafluoroborate or (1-cyano-2-ethoxy-2-oxoethylidenaminooxy)dimethylamino-morpholino-carbenium hexafluorophosphate in the presence of an amine such as triethyl amine, diisopropyl amine or diisoproyl ethylamine and in a solvent such as dichloromethane, an ethereal solvent such as tetrahydofuran, a polar aprotic solvent such as dimethylformamide, or a more environmentally acceptable and sustainable alternative such as dimethyl carbonate, ethyl acetate or methyltetrahydrofuran.

In one embodiment, in order to improve the dehepaticability of the compound disclosed in the present invention for the required physiological effect, a “pro-drug” of the same can be made available. For example, the pro-drug can be a obtained by conjugation of the parent drug with polyethylene glycol (PEG).

In one embodiment, the compounds in the present invention could exist as enantiomers, diastereomers, geometric isomers, racemates, tautomers, rotamers, atropisomers or metabolites.

In some embodiments, the compounds have at least one fluorine atom incorporated at or adjacent to a site that is susceptible to metabolism. The fluorine atom can be part or adjacent to a substituent group as defined.

In one embodiment, some compounds disclosed in the invention can be “neutral antagonists”. These agents are said to have no effect on intrinsic receptor activity at least in certain test systems. However, these agents may be able to block receptor binding and activation, usually by a competitive agonist.

In some embodiments, it would be desirable to have antagonists that exhibit essentially no CB1 receptor activity and which block or significantly reduce receptor activation by a suitable agonist. It would be further desirable to have neutral antagonists of the CB1 receptor that can be used to prevent, treat, or reduce the severity of symptoms of certain medical conditions. It would be especially desirable to have neutral antagonists that exhibit no or minimal side-effects in vivo.

In another embodiment, the compound disclosed in the present invention may act preferentially at the CB1 receptors located in the periphery. In certain embodiments, the compounds do not penetrate the blood-brain-barrier, have restricted penetration or have slow penetration. In certain embodiments, peripherally acting compounds that are excluded from the CNS may have advantages over centrally acting compounds, for example, reduced psychotropic adverse effects.

A compound acting on the CB1 receptors located in the periphery could be behave either as a neutral antagonist, an inverse agonist or a partial antagonist.

A compound acting on the CB1 receptors located centrally, could behave as a neutral antagonist, an inverse agonist or a partial antagonist.

In another embodiment, the compounds disclosed in the invention could act either as inverse agonists with no or reduced side effects. In other embodiments, the compounds could act as partial agonists with no or reduced side effects.

The CB1 cannabinoid receptor binding affinities (Ki) for some of the compounds disclosed in the invention range between 0.1 nM and less than 100 nM. For example, CB1 cannabinoid receptor binding affinity (Ki) for compound 1 is 1 nM and the CB2 cannabinoid receptor binding affinity (Ki) is greater than 1000 nM. The CB1 selectivity for some of the compounds range from 5 to greater than 5000.

The compounds of the present invention can be administered by a variety of known methods, including orally, rectally, or by parenteral routes (e.g., intramuscular, intravenous, subcutaneous, nasal or topical). The form in which the compounds are administered will be determined by the route of administration. Such forms include, mixtures for oral and rectal administration, liquid mixtures for oral, intravenous, intramuscular, subcutaneous, ocular, intranasal, inhalation-based and transdermal administration and slow microcarriers for rectal, intramuscular or intravenous administration. Suitable physiologically acceptable vehicles for inclusion with mixtures include, for example, saline, sterile water, Ringer's solution and isotonic sodium chloride solutions. The specific dosage level of active ingredient will depend upon a number of factors, including, for example, biological activity of the particular preparation, age, body weight, sex and general health of the individual being treated.

The disclosure also provides a drug composition formulated for inhalation comprising a surface-active agent or an exogenous surfactant and a compound disclosed in the invention to provide a pulmonary active drug. The surface-active agent has an affinity for the human alveolar/gas interface and comprises at least a portion of a mammalian pulmonary surfactant or a mimic thereof. In certain embodiments, this exogenous surfactant may act as a carrier of the pulmonary active drug and in some embodiments the carrier will facilitate the drug to reach the edematous areas of the lung while improving the bioavailability.

The disclosure also provides a method of treating a subject suffering from or at risk of suffering from a lung disease such as fibrosis, neonatal respiratory distress syndrome (RDS) and acute RDS (ARDS) that may occur due to COVID-19 comprising administering to the subject a mixture comprising a drug for lung treatment and a surface active agent by inhalation in an amount effective to induce a drug effect in the lungs.

In certain embodiments, a compound may be formulated with lavage-derived surfactants, minced lung surfactants or synthetic surfactants and may contain in various ratios a combination of dipalmitoyl phosphatidylcholine, unsaturated phosphatidylcholine, phosphatidylglycerol, other phospholipids including phosphatidylserine, phosphatidylethanolamine, phosphatidylinositol, lyso-phosphatidylcholine; and sphingomyelin, a neutral lipid such as Cholesterol and free fatty acids, in combination with surfactant proteins SP-B and SP-C. In some cases, synthetic additives such as Hexadecanol, Tyloxapol recombinant SP-C, KL4 peptide, SP-B and SP-C analogs that can mimic the native peptide sequence can be used instead.

In another embodiment, the compounds of the present disclosure can also comprise isotopes at one or more of their atoms. For example, the compounds can be radiolabeled with isotopes, such as ²H (deuterium written as D), ³H (tritium written as T), ¹¹C (carbon-11), ¹³C (carbon-13), ¹⁴C (carbon-14), ¹⁵O (oxygen-15), ¹⁷O (oxygen-17), ¹⁸O (oxygen-18), ¹³N (nitrogen-13), ¹⁵N (nitrogen-15), ¹⁸F (fluorine-18), ⁷⁵Br (bromine-75), ⁷⁶Br (bromine-76), ⁷⁷Br (bromine-77), ⁸²Br (bromine-82), ¹²³I (iodine-123), ¹²⁴I (iodine-24), ¹²⁵1I (iodine-125) or ¹³¹I (iodine-131), ³⁶Cl (chlorine-36), ³⁵S (sulphur-35), ⁶⁴Cu (Copper-64), ⁶⁸Ga (Gallium-68), ⁸⁹Zr (Zirconium-89), ^{99m}Tc (Technetium-99m), ¹¹¹In (Indium-111) and ¹⁷⁷Lu (Lutetium-35). The present disclosure encompasses all isotopic variations of the described compounds, whether natural or unnatural, radioactive or not.

An isotope is one of two or more species of the same element. Each isotope of an element will have the same number of protons in its nucleus, the same atomic number and the same position in the Periodic Table. However, each isotope of that element will have a different number of neutrons in its nucleus and therefore a different mass than other isotopes of that species. The term nuclide is sometimes used synonymously with the term isotope. As used herein a natural isotope has an atomic mass corresponding most closely with the atomic mass shown for that element in the Periodic Table. As used herein an unnatural isotope has an atomic mass that is further removed from the atomic mass shown for that element in the Periodic Table than the natural isotope. For example, protium (hydrogen-1 or ¹H) is the natural isotope of hydrogen and deuterium (hydrogen-2 or ²H) and tritium (hydrogen-3 or ³H) are all unnatural isotopes of hydrogen.

In some embodiments, the compounds have at least one isotope atom incorporated at or adjacent to a site that is susceptible to metabolism. The isotope atom can be part or adjacent to a substituent group as defined.

In some embodiments, the compounds have at least one deuterium atom incorporated at or adjacent to a site that is susceptible to metabolism. The deuterium atom can be part or adjacent to a substituent group as defined.

In a particular embodiment, some of the halogen containing analogs, for example those analogs comprising iodide and fluoride, are potential radioactive probes for imaging in vivo the distribution of cannabinoid receptors. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

Some of the radioactive isotope containing analogs have potential as radiopharmaceutical analogs (disclosed analogs that have been labeled with radioactive isotopes). These radiopharmaceuticals can be administered to individuals or animals and the emitted radiation can be measured. The majority of these diagnostic tests involve the formation of an image using a camera suitable to detect the emitted radiation. Positron emission tomography (PET) is one nuclear medicine tomographic imaging technique, which produces a three-dimensional image or map of functional processes in a patient's body. To conduct the PET scan, a short-lived radiopharmaceutical analog that decays by emitting a positron is administered into the subject (usually by injection into the blood stream). There is a waiting period while the radiopharmaceutical analog becomes concentrated in tissues of interest such as a cannabinoid receptor. After the waiting period the patient is placed in an imaging scanner. The scanner collects multiple images and a computer is used to apply an algorithm to the multiple images and provide a three dimensional image. Single photon emission computed tomography (SPECT) is another nuclear medicine tomographic imaging technique. To conduct the SPECT scan, a short-lived radiopharmaceutical analog that decays to produce a gamma ray is administered into the subject. There is a waiting period while the radiopharmaceutical analog becomes concentrated in tissues of interest such as a cannabinoid receptor. After the waiting period the patient is placed in an imaging scanner and SPECT imaging is performed by using a gamma camera to acquire multiple two-dimensional images from multiple angles. A computer is then used to apply an algorithm to the multiple images to provide a three-dimensional image.

Some of the radioactive isotope containing analogs have potential as theranostics or theragnostics wherein the compounds disclosed in the invention can be used as combinations as therapeutics and diagnostics.

As used herein, an “inflammatory condition” refers to a condition involving hepatic, renal, pulmonary disease or prostrate.

In a particular embodiment, the hepatic disease may be alcoholic or non-alcoholic steatohepatitis.

In a particular embodiment, the hepatic disease may be acute or chronic viral hepatitis including hepatitis C virus.

In a particular embodiment, the hepatic disease may be alcoholic or non-alcoholic cirrhosis.

In a particular embodiment, the hepatic disease may be liver cancer.

In a particular embodiment, the pulmonary disease may be idiopathic, genetic, radiation-induced or environmental agent induced, including chemicals.

In a particular embodiment, the renal disease may be chronic kidney disease including diabetic nephropathy In a particular embodiment, the “inflammatory condition” can be relate to fibrosis of the lung, liver or kidney.

In a particular embodiment, fibrosis can be a condition related to the liver, lung, kidney or prostrate.

In a particular embodiment, the condition can be prostatic fibrosis and related inflammatory conditions including lower urinary tract symptoms.

In a particular embodiment, the inflammatory condition can be immune inflammatory disease such as benign prostatic hyperplasia. An “animal” refers to, for example nonhuman-primates such as squirrel monkeys, rhesus monkeys, marmosets, baboons, veterinary animals, such as rodents, dogs, cats, horses and the like, and farm animals, such as cows, pigs and the like.

Several embodiments of the invention are further described in the Specification. It will be recognized that one or more features of any embodiments disclosed herein may be combined and/or rearranged within the scope of the invention to produce further embodiments that are also within the scope of the invention.

Still other objects and advantages of the invention will become apparent by those of skill in the art from the disclosure herein, which are simply illustrative and not restrictive. Thus, other embodiments will be recognized by the ordinarily skilled artisan without departing from the spirit and scope of the invention.

The disclosure is further illustrated by the following examples, which are not to be construed as limiting this disclosure in scope or spirit to the specific procedures described in this disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Compound Synthesis

All reagents and solvents used for chemical synthesis were purchased from Sigma-Aldrich, TCI Chemicals, Fisher Scientific, Acros or Alfa Aesar. The palladium catalysts were purchased from Sigma-Aldrich or TCI Chemicals. 1H NMR (500 MHz) was recorded on a Varian Inova spectrometer. Chemical shifts (δ) are reported in parts per million and are referenced to CDCl₃ for 7.26. Multiplicities are indicated as br (broadened), s (singlet), d (doublet), t (triplet) or m (multiplet). Coupling constants (J) are reported in hertz (Hz). Thin layer chromatography (TLC) was performed on Merck-Millipore 210-270 μm TLC silica gel plates, (60 Å) and coated with a F254 fluorescent indicator. Flash column chromatography was performed on a Biotage Isolera Spektra system with UV collections at 254 and 280 nm using Luknova flash columns preloaded with normal phase silica gel (50 μm). All moisture sensitive reactions were performed under an atmosphere of high-purity argon while using oven-dried glassware. The intermediates and final compounds were characterized using a combination of 1H NMR and LC/MS techniques. The LC/MS analysis (11 minute run) was performed as using a Waters MicroMass ZQ system (electrospray ionization mode) equipped with a Waters 2525 binary gradient module, a Waters 2996 photodiode array detector, a Waters 2424 ELS detector, two Waters 515 HPLC pump, a fluidics organizer and a pump control module II. Compounds were analyzed with gradient elution using acetonitrile/water as the mobile phase and an XTerra MS C18 or an XTerra MS C8, 4.6 mm×50 mm column (5 μm). Melting-points were recorded on a Fisher Scientific apparatus.

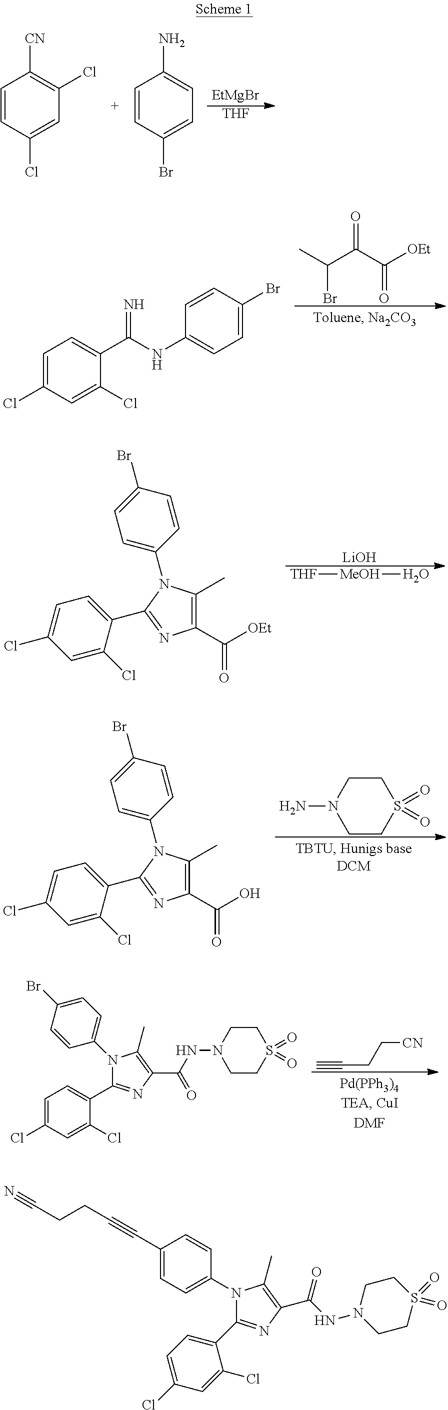

### Example 1

Method 1

### Step A1

### N-(4-bromophenyl)-2,4-dichlorobenzimidamide

To a magnetically stirred solution of EtMgBr (3.3 mL, 3M in diethyl ether, 10 mmol) in THF (30 mL) 4-bromoaniline (1.72 g, 10 mmol) was slowly added portion wise. After the solution was stirred for 30 min., 2,4-dichlorobenzonitrile (1.72 g, 10 mmol) was added. The resulting solution was stirred at room temperature (RT) overnight. The reaction mixture was quenched with water and extracted with ethyl acetate. The combined extracts were dried over anhydrous MgSO₄, filtered and evaporated under reduced pressure to give the benzimidamide as an off-white solid (2.45 g, 71.2%).

### Step B1

### Ethyl 1-(4-bromophenyl)-2-(2,4-dichlorophenyl)-5-methyl-1H-imidazole-4-carboxylate

To a magnetically stirred solution of above amidine intermediate from step A1 (2.45 g, 7 mmol) in 30 mL anhydrous toluene were added ethyl 3-bromo-2-oxobutanoate (1.48 g, 7 mmol) and Na₂CO₃ (0.74 g, 7 mmol). The contents were stirred at 100° C. for 12 hours. The reaction was brought to RT. The reaction mixture was quenched with water and extracted with ethyl acetate. The combined extracts were dried over anhydrous MgSO₄, filtered and evaporated under reduced pressure. Purification by column chromatography gave the ester as pale white solid (1.5 g, 46.4%).

### Step C1

### 1-(4-bromophenyl)-2-(2,4-dichlorophenyl)-5-methyl-1H-imidazole-4-carboxylic acid

The ester (10 g, 19.9 mmoles) obtained from step B1 was taken in 500 ml single neck flask and to it 300 ml of 7:2:1 mixture of THF-methanol-water along with solid lithium hydroxide (2.5 g, 104.6 mol) was added. The mixture was refluxed for 12 hours. The solvents were removed totally and to the residue 200 ml of DCM was added. To that 100 ml of water was added and the mixture was acidified to pH˜2 using concentrated HCl. The organic layer was separated, washed with 100 ml of brine, dried over sodium sulphate and concentrated to give the acid. This was taken directly to the next step (9.4 g, 100%).

### Step D1

### 1-(4-bromophenyl)-2-(2,4-dichlorophenyl)-N-(1,1-dioxidothiomorpholino)-5-methyl-1H-imidazole-4-carboxamide

The acid (7.5 g, 15.8 mmol) obtained from step C1 was taken in a 500 ml single neck flask equipped with a nitrogen inlet and to it 200 ml of DCM, 4-aminothiomorpholine-1,1-dioxide (2.61 g, 17.4 mmol), TBTU (5.59 g, 17.4 mmol) and DIPEA (2.25 g, 17.4 mmol) were added and the contents were stirred for 1 hour. To the reaction mixture, 100 ml of water was added and the contents were acidified to pH-2 using concentrated HCl. The organic layer was separated, washed with brine, dried over sodium sulphate and concentrated to give the amide (3 g, 31.2%).

### Step E1

### 1-(4-(4-cyanobut-1-yn-1-yl)phenyl)-2-(2,4-dichlorophenyl)-N-(1,1-dioxidothiomorpholino)-5-methyl-1H-imidazole-4-carboxamide (compound 1)

To a stirred solution of amide obtained from step D1 (1, 1.1 g, 2 mol) in DMF (30 ml), under argon was added 4-cyano-1-butyne (888 mg, 6 mmol), and Hünig's base (1.9 g, 20 mmol). The reaction mixture was degassed by introducing a steady stream of argon into the solution for 5 min and to this was added tetrakis(triphenylphosphine)palladium (0) (231 mg, 10 mol %) and CuI (76 mg, 20 mol %). The resulting mixture was stirred for 3 h at room temperature. The solvent from the reaction mixture was removed in vacuo at 70 deg C. and the residue was dissolved in dichloromethane (100 ml) and washed with deionized water (2×˜50 mL). The organic layer was separated, dried over anhydrous MgSO₄, filtered and the filtrate was removed in vacuo. The residue obtained was purified by flash column chromatography on silica gel (n-hexane/AcOEt=1/1) to provide 2 as a white solid (511 mg, 45%); m.p 176-178 deg C.;

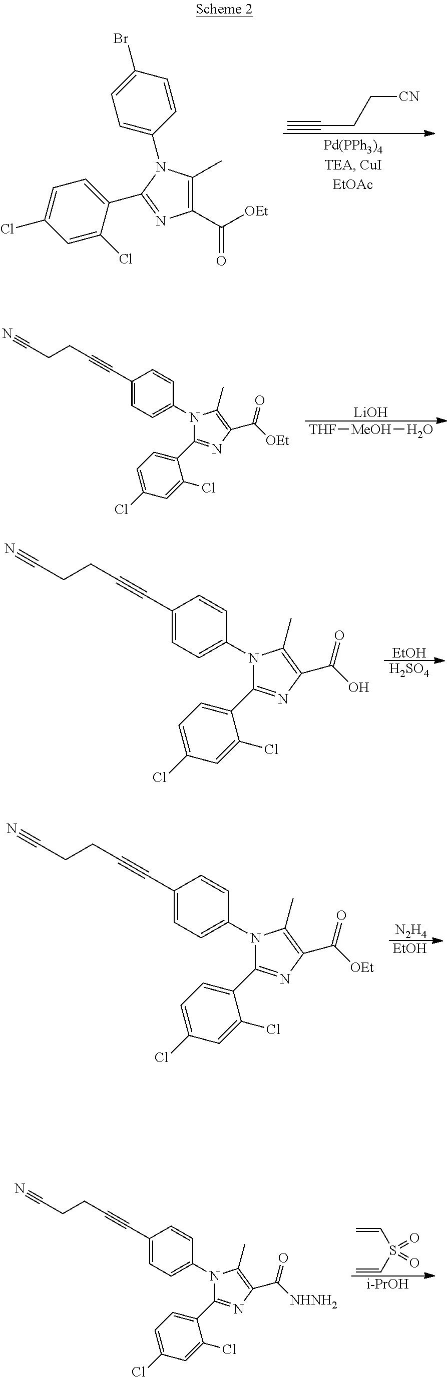

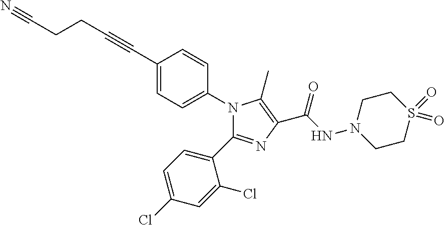 [SMILES: Cc1c(C(=O)NN2CCS(=O)(=O)CC2)nc(-c2ccc(Cl)cc2Cl)n1-c1ccc(C#CCCC#N)cc1]

Method 2

### Step A2

### Ethyl-1-(4-(4-cyanobut-1-yn-1-yl)phenyl)-2-(2,4-dichlorophenyl)-5-methyl-1H-imidazole-4-carboxylate

To a stirred solution of bromo ester obtained from method 1, step B1 (18 g) in ethyl acetate (400 ml), under argon was added 4-cyano-1-butyne (10 g), and diisopropyl amine (7.8 ml). The reaction mixture was degassed by introducing a steady stream of argon into the solution for 5 min and to this was added tetrakis(triphenylphosphine)palladium (0) (1.8 g) and copper(I) iodide (400 mg). The resulting mixture was refluxed for 3 h. The solvent was washed with deionized water (2×˜100 mL). The organic layer was separated, dried over anhydrous MgSO₄, filtered and the filtrate was removed in vacuo. The residue obtained was carried to the next reaction without further purification.

### Step B2

### 1-(4-(4-Cyanobut-1-yn-1-yl)phenyl)-2-(2,4-dichlorophenyl)-5-methyl-1H-imidazole-4-carboxylic acid

The ester (10 g, 19.9 mmoles) obtained from step A2 was taken in 500 ml single neck flask and to it 300 ml of 7:2:1 mixture of THF-methanol-water along with solid lithium hydroxide (2.5 g, 104.6 mol) was added. The mixture was refluxed for 12 hours. The solvents were removed and to the residue 200 ml of DCM was added. To that 100 ml of water was added and the water layer containing the lithium salt of the product was separated. The water layer was repeatedly washed with DCM (2×100), the water layer separated and was acidified to pH˜2 using concentrated HCl. The organic layer was separated, washed with 100 ml of brine, dried over sodium sulphate and concentrated to give the purified acid free of any impurities. This purified acid was dried over MgSO₄, filtered and the filtrate was passed through a short bed of silica gel, and the organic layer was concentrated and the residue was taken directly to the next step (9.4 g, 100%).

### Step C2

### Ethyl-1-(4-(4-cyanobut-1-yn-1-yl)phenyl)-2-(2,4-dichlorophenyl)-5-methyl-1H-imidazole-4-carboxylate

The acid (7.5 g, 15.8 mmol) obtained from step B2 was taken in a 500 ml single neck flask equipped with a nitrogen inlet and to it 200 ml of anhydrous ethyl alcohol. To this was added few drops of sulfuric acid and the mixture was refluxed for 12 hours. The solvents were removed, and the residue was dissolved in dichloromethane (100 ml) and washed with deionized water (2×-100 mL). The organic layer was separated, dried over anhydrous MgSO₄, filtered and the solvent was evaporated in vacuo to provide the ester as an off-white solid (7 g, 87%).

### Step D2

### 1-(4-(4-Cyanobut-1-yn-1-yl)phenyl)-2-(2,4-dichlorophenyl)-5-methyl-1H-imidazole-4-carbohydrazide

The ester (7 g, 15.4 mol) obtained from step C2 was taken in a 500 ml single neck flask equipped with a nitrogen inlet and to it 50 ml of anhydrous ethyl alcohol followed by 20 g of hydrazine hydrate are added and the mixture is heated at reflux for 3 hours. The reaction medium is concentrated in vacuo with 10% residual solvent remaining. The combined residue is dissolved in dichloromethane (150 ml) and washed with deionized water (2×-100 mL). The organic layer was separated, dried over anhydrous MgSO₄, filtered and the filtrate was removed in vacuo. The residue obtained was purified by crystallization from ethyl acetate and hexane to provide the hydrazide as a white solid (5.2 g, 76%).

### Step E2

### 1-(4-(4-cyanobut-1-yn-1-yl)phenyl)-2-(2,4-dichlorophenyl)-N-(1,1-dioxidothiomorpholino)-5-methyl-1H-imidazole-4-carboxamide (compound 1)

To a solution of the hydrazide from step D2 (5 g, 11.4 mmol) in iPrOH (6 mL) was added divinyl sulfone (1.35 g, 11.4 mmol) dropwise and the contents were stirred overnight at room temperature. The solids obtained were filtered and washed with iPrOH (2×25 ml) and dried to provide the title compound (3.5 g, 55%).

¹H NMR (500 MHz, CDCl₃) δ ppm 8.34 (s, 1H), 7.42 (d, J=8.30 Hz, 2H), 7.34 (s, 1H), 7.22-7.29 (m, 2H), 7.03 (d, J=8.30 Hz, 2H), 3.45-3.64 (m, 4H), 3.27 (m, 4H), 2.79 (t, J=1.00 Hz, 2H), 2.65 (t, J=1.00 Hz, 2H), 2.47 (s, 3H); ES m/z 556.09 (M⁺+H), t_{R} 4.5 min.

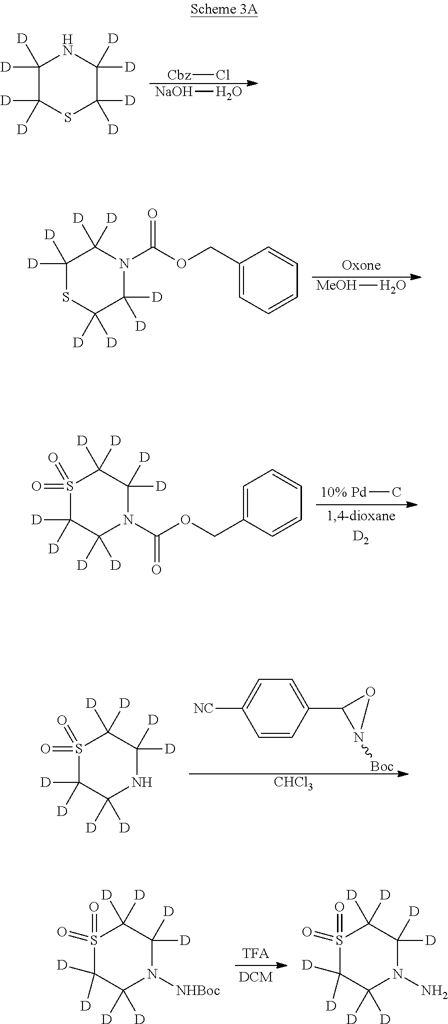

### Example 2

### Step A

### Benzyl thiomorpholine-4-carboxylate-d8

To a solution of thiomorpholine-2,2,3,3,5,5,6,6-d8 (2 g) prepared as in WO2008070619, taken in 1 N NaOH (11.6 mL) was added benzyl chloroformate (1.66 mL) under ice water cooling and the mixture was stirred at ambient temperature for 2 hours. The solution was neutralized with 1 N HCl and extracted with EtOAc twice. The combined organic layer was washed with water and brine, dried over MgSO₄ and evaporated in vacuo. The residue was purified by silica gel column chromatography to give benzyl thiomorpholine-4-carboxylate-d8 as a colorless Solid (4.8 g).

### Step B

### Benzyl thiomorpholine-4-carboxylate-2,2,3,3,5,5,6,6-d8 1,1-dioxide

To a solution of benzyl thiomorpholine-4-carboxylate-d8 (4.8 g) in methanol (30 mL) and H2O (20 mL) was added oxone (16.2 g) under ice water cooling and the mixture was stirred at ambient temperature for 2 hours. The solution was evaporated in vacuo and partitioned between EtOAc and water. The aqueous layer was extracted with EtOAc. The combined organic layer was washed with water and brine, dried over MgSO₄ and evaporated in vacuo. The residue was purified by silica gel column chromatography eluting with a mixture of hexane and EtOAc to give the title compound (3.8 g).

### Step C

### Thiomorpholine 1,1-dioxide-d9

To a solution of benzyl thiomorpholine-4-carboxylate-2,2,3,3,5,5,6,6-d8 1,1-dioxide (3.8 g) in methanol (32 mL) and 1,4-dioxane (8 mL) was added Palladium, 10 wt. % on activated carbon (380 mg) at ambient temperature. The mixture was stirred at ambient temperature for 4 hours under an environment of D2. The mixture was filtered and evaporated in vacuo to give title compound (2.22 g).

### Step D

### tert-Butyl (1,1-dioxidothiomorpholino-2,2,3,3,5,5,6,6-d8)carbamate

To a solution of thiomorpholine 1,1-dioxide-d9 (2 g) in CHCl₃ (20 mL) was treated at 0 deg C. by a solution of tert-butyl 3-(4-cyanophenyl)-1,2-oxaziridine-2-carboxylate (3.4 g) in CHCl₃ (20 mL). At the end of the addition the cooling bath was removed, and the contents were allowed to stir overnight. The solvent was evaporated, and the residue obtained was carried to the next step directly.

### Step E

### 4-Aminothiomorpholine 1,1-dioxide-2,2,3,3,5,5,6,6-d8

To a stirred solution of crude tert-butyl (1,1-dioxidothiomorpholino-2,2,3,3,5,5,6,6-d8)carbamate obtained in step D in dichloromethane (25 ml) at 0° C. was added trifluoroacetic acid (15 ml). After stirred at room temperature for 1 hour, the mixture was evaporated to give an oil. Diethyl ether (100 ml) was added, and the white precipitate was separated. The upper solution was then removed by decanting. This procedure was repeated three times to ensure complete removal of excess trifluoroacetic acid. The remaining white solids were dried in vacuo to give the title compound in quantitative yield.

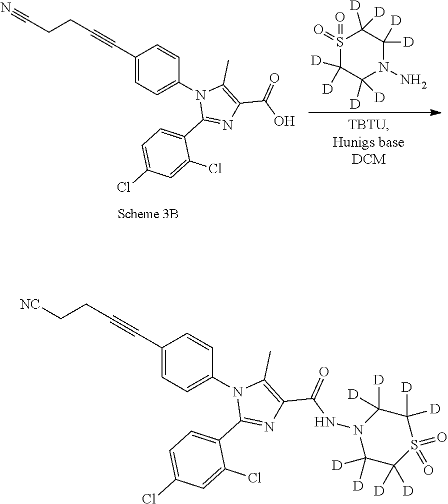 [SMILES: Cc1c(C(=O)O)nc(-c2ccc(Cl)cc2Cl)n1-c1ccc(C#CCCC#N)cc1 ; [2H]C1([2H])N(N)C([2H])([2H])C([2H])([2H])S(=O)(=O)C1([2H])[2H] ; [2H]C1([2H])N(NC(=O)c2nc(-c3ccc(Cl)cc3Cl)n(-c3ccc(C#CCCC#N)cc3)c2C)C([2H])([2H])C([2H])([2H])S(=O)(=O)C1([2H])[2H]]

### Step F

### 1-(4-(4-Cyanobut-1-yn-1-yl)phenyl)-2-(2,4-dichlorophenyl)-N-(1,1-dioxidothiomorpholino-2,2,3,3,5,5,6,6-d8)-5-methyl-1H-imidazole-4-carboxamide

The acid (7.5 g, 15.8 mmol) obtained from example 1, method 2, step B2 was taken in a 500 ml 1 neck flask equipped with a nitrogen inlet and to it 200 ml of DCM, 4-aminothiomorpholine 1,1-dioxide-2,2,3,3,5,5,6,6-d8 (2.61 g, 16.4 mmol), TBTU (5.59 g, 17.4 mmol) and DIPEA (2.25 g, 17.4 mmol) were added and the contents were stirred for 1 hour. To the reaction mixture, 100 ml of water was added, and the contents were acidified to pH-2 using concentrated HCl. The organic layer was separated, washed with brine, dried over sodium sulphate and concentrated to give the title compound (3 g); ES m/z 554.14 (M⁺+H), tR 4.5 min.

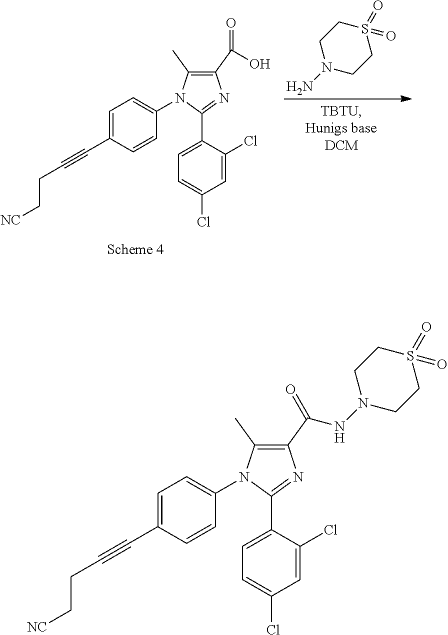 [SMILES: Cc1c(C(=O)O)nc(-c2ccc(Cl)cc2Cl)n1-c1ccc(C#CCCC#N)cc1 ; NN1CCS(=O)(=O)CC1 ; Cc1c(C(=O)NN2CCS(=O)(=O)CC2)nc(-c2ccc(Cl)cc2Cl)n1-c1ccc(C#CCCC#N)cc1]

### Example 3

The acid (15.8 mmol) obtained from step B2 scheme 2 was taken in a 500 ml 1 neck flask equipped with a nitrogen inlet and to it 200 ml of DCM, 4-aminothiomorpholine 1,1-dioxide (16.4 mmol), TBTU (17.4 mmol) and DIPEA (17.4 mmol) were added and the contents were stirred for 1 hour (scheme 4). To the reaction mixture, 100 ml of water was added, and the contents were acidified to pH-7 using 2N HCl. The organic layer was separated, washed with brine, dried over sodium sulphate and concentrated to provide 1-(4-(4-cyanobut-1-yn-1-yl)phenyl)-2-(2,4-dichlorophenyl)-N-(1,1-dioxidothiomorpholino)-5-methyl-1H-imidazole-4-carboxamide in 79% yields.

### Example 4

### Membrane Preparations from Tissue Culture Sources

HEK293 cells expressing hCB1, hCB2 or mCB2 receptor are used for membrane preparations according to the method described in J Neurochem 1999, 72, (5), 2032-8, herein incorporated by reference in its entirety. The resulting pellet is resuspended in 10 mM Tris-chloride, pH 7.4 with 5 mM MgCl₂ and 2 mM EDTA (TME), and stored at −80° C. for no longer than two months. Protein content is assayed by using the Bio-Rad DC protein assay according to the manufacturer's protocol.

### Example 5

### Membrane Preparations from Tissue Sources

Frozen rat brains (CB1 source) are obtained from Pel-Freeze Biologicals (Rogers, Ak.) and stored at −80° C. until use. Membranes are prepared according to the method described in Brain Res 1981, 226, (1-2), 107-18 and adapted as previously reported in J Med Chem 1994, 37, (23), 3867-70 and Life Sci 1995, 56, (23-24), 1957-62; each herein incorporated by reference in its entirety.

### Example 6

### rCB1, hCB2, and mCB2 Binding Assays

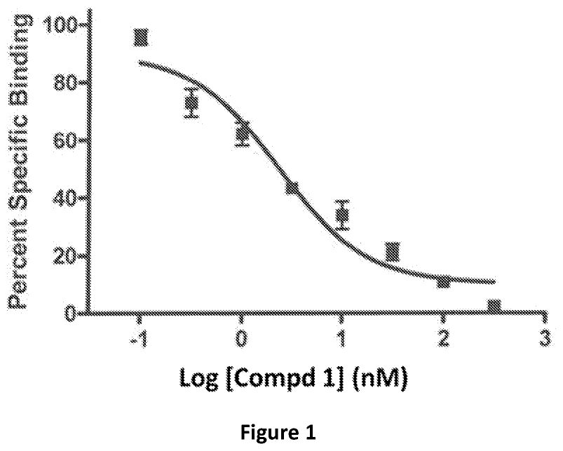

The compound was tested for its ability to bind to CB1 and CB2 receptors using rat brain or HEK293 cell membranes expressing hCB2 and mCB2 membrane preparations, respectively, as described in J Med Chem 1999, 42, (4), 769-776, J Med Chem 1994, 37, (23), 3867-70 and Life Sci 1995, 56, (23-24), 1957-62 (each herein incorporated by reference in its entirety) via competition-equilibrium binding using [³H]CP-55,940. The results were analyzed using nonlinear regression to determine the actual IC₅₀ of the ligand (Prizm by GraphPad Software, Inc.) and the Ki values are calculated from the IC₅₀ as described in Biochemical Pharmacology 1973, 22, (23), 3099-3108; herein incorporated by reference in its entirety. Competition binding for Compound 1 toward CB1 is shown in FIG. 1 (Ki˜1 nM).

### Example 7

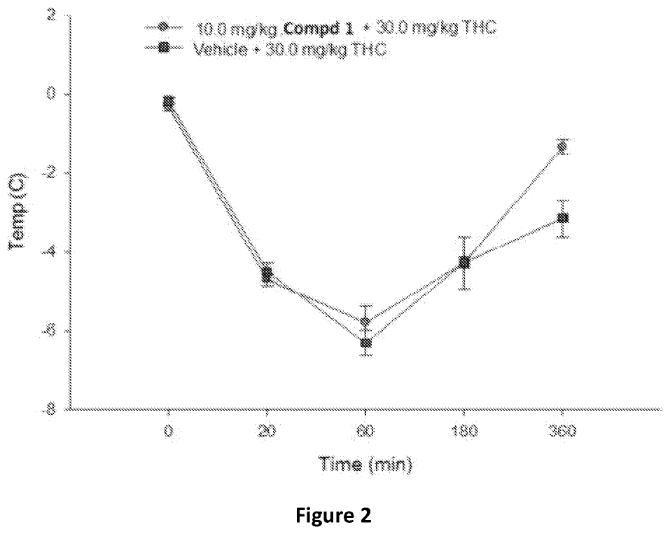

Male CD-1 mice (Charles River, Wilmington, Mass., USA) aged 4-6 weeks weighing between 25-35 gm were chosen for in vivo CB1 antagonism testing. The mice were housed 4 per cage with free access to food and water. The temperature of the vivarium was 25° C. with a non-reversed 12 hrs light/dark cycle (lights on 7 am). Mice were habituated to the vivarium for at least one week before any experiments were performed. All experimental procedures were pre-approved by The Animal Care and Use Committee of Northeastern University, Boston, Mass., USA. The “Principles of Animal Laboratory Care” (National Institute of Health 1996) was followed. The test compound (10 mg/kg) was dissolved in 4% dimethyl sulfoxide, followed by 8% Tween-80 and 8% propylene glycol. Saline was added slowly to this solution with sonication prior to administration. Δ⁹-THC was initially dissolved in 2% dimethyl sulfoxide, followed by 4% Tween-80 and 4% propylene glycol. Saline was added slowly to this drug solution with sonication prior to administration. All administrations were i.p. at a volume of 10 ml/kg. Temperature recordings took place during the light cycle between 11 am-7 μm. The testing room was kept at ambient temperatures varying between 22° C.-24° C. The animals were acclimated to the test room for 30 min before the 1st recording (baseline) followed by a 2nd recording 15 min later (time point zero), immediately after which the CB1 R antagonist was administered. Δ⁹-THC (30 mg/kg) was given 15 min later and the 3rd recording occurred 20 min thereafter, i.e., time point 35 min. The remaining recordings were taken at 75, 195 and 375 min post antagonist administration. Core temperature was measured using a rectal probe of a digital laboratory thermometer, RET-3-ISO, type T thermocouple (Physitemp Instruments Inc., Clifton, N.J.). The probe was inserted 2 cm into the rectum for ≈20 to 30 seconds at which time the rectal temperature readings had stabilized. FIG. 2 shows the inability of 10 mg/kg of compound 1 to antagonize the hypothermic effects induced by 30 mg/kg of the CB1 agonist Δ⁹-THC, indicating that Compound 1 is largely peripheral and is excluded from the CNS.

### Example 8

Timed pregnant female C57BL/6 mice (Jackson Laboratories, USA) were obtained and housed with a cardboard hut for birthing and nursing of pups (enrichment), in an AAALAC-accredited vivarium, prefilled with corn cob bedding on a 12-hour light-dark at 68-74° F. and 30-70% humidity. Pregnant female mice were fed water and a standard rodent diet (Teklad irradiated 2918) ad libitum consisting of 18% protein, 5% fat, and 5% fiber. Three days post birth, pups from all litters received streptozotocin (STZ), i.p. at 200 mg/kg, to induce a mild to moderate hyperglycemia and insulin insufficiency. Random blood glucose was measured at weaning when pups were 25 days old. At weaning, mice were fed water and a standard rodent diet (Teklad irradiated 2918) ad libitum consisting of 18% protein, 5% fat, and 5% fiber. At 4 weeks of age, male mice were randomized into 7 study groups based on a random blood glucose (RBG) between 200-750 mg/dl and were switched from the standard rodent diet to the modified high fat diet (HFD), Research Diets D09100310 for the remainder of the study. Following 3 weeks of HFD feeding, test article and vehicle control dose administration was initiated and continued for up to 9 weeks. Body weights were measured at least twice a week from dose initiation through study end/termination. HFD was changed weekly with the old food being discarded. Animals had random blood glucose measured every 3 weeks and at termination. Clinical observations were performed daily from receipt/birth and continued through study end. Compound 1 and rimonabant were stored at −20° C. prior to drug preparation. Following three weeks of modified diet feeding, dose initiation of the compounds began and continued for up to 9 weeks. Test compounds were dissolved in 2% dimethyl sulfoxide, followed by 8% Tween-80 and 8% propylene glycol. Saline was added slowly to this solution with sonication prior to administration. Animals were dosed at 10 mg/kg via oral gavage (PO), daily, for up to 9 weeks.

Livers were scored for both NAS and NASH parameters. Steatosis, lobular inflammation, ballooning degeneration and fibrosis were observed in both H&E and PSR stained sections of the control and the therapeutic treatment groups. Fibrosis was observed across all study groups. Following 9 weeks of treatment, the fibrosis scores of Compound 1 and rimonabant treatment groups were found to be statistically significant when compared to the control group. Steatosis was observed in all study groups and decreased following 9 weeks of treatment with both rimonabant and Compound 1. Lobular inflammation was present across all NASH study groups thus indicating inflammatory cell infiltration. Following 9 weeks of treatment with Compound 1, lobular inflammation was reduced. Animals treated with Compound 1 showed a near significant reduction in lobular inflammation. The NAFLD/NAS activity score was found to be lowered across all treatment groups when compared to the control group; The NASH scores were lowered in all treatment groups. Pharmacological intervention using Compound 1 markedly attenuated or delayed the inflammation and fibrosis and increased animal survival as compared to rimonabant.

In more preferred embodiments, the compounds of claim 1 are selected from
1-(4-chlorophenyl)-2-(2,4-dichlorophenyl)-N-(1,1-dioxidothiomorpholino)-5-methyl-1H-imidazole-4-carboxamide
ethyl 4-(2-(2,4-dichlorophenyl)-4-((1,1-dioxidothiomorpholino)carbamoyl)-5-methoxy-1H-imidazol-1-yl)benzoate
ethyl 4-(2-(4-chloro-2-fluorophenyl)-4-((1,1-dioxidothiomorpholino)carbamoyl)-5-methoxy-1H-imidazol-1-yl)benzoate
2-(2,4-dichlorophenyl)-N-(1,1-dioxidothiomorpholino)-1-(4-iodophenyl)-5-methyl-1H-imidazole-4-carboxamide
ethyl 4-(2-(2,4-dichlorophenyl)-4-((1,1-dioxidothiomorpholino)carbamoyl)-5-methyl-1H-imidazol-1-yl)benzoate
ethyl 4-(2-(2,4-dichlorophenyl)-4-((1,1-dioxidothiomorpholino)carbamoyl)-5-ethyl-1H-imidazol-1-yl)benzoate
ethyl 4-(2-(2,4-dichlorophenyl)-4-((1,1-dioxidothiomorpholino)carbamoyl)-5-(hydroxymethyl)-1H-imidazol-1-yl)benzoate
2-(2,4-dichlorophenyl)-N-(1,1-dioxidothiomorpholino)-1-(4-(4-isothiocyanatobut-1-yn-1-yl)phenyl)-5-methyl-1H-imidazole-4-carboxamide
4-(4-(2-(2,4-dichlorophenyl)-4-((1,1-dioxidothiomorpholino)carbamoyl)-5-methyl-1H-imidazol-1-yl)phenyl)but-3-yn-1-yl nitrate
2-(2,4-dichlorophenyl)-N-(1,1-dioxidothiomorpholino)-1-(4-(4-isothiocyanatobut-1-yn-1-yl)phenyl)-5-methoxy-1H-imidazole-4-carboxamide
1-(4-(4-cyanobut-1-yn-1-yl)phenyl)-2-(2,4-dichlorophenyl)-N-(1,1-dioxidothiomorpholino)-5-methyl-1H-imidazole-4-carboxamide
2-(4-chloro-2-fluorophenyl)-1-(4-(4-cyanobut-1-yn-1-yl)phenyl)-N-(1,1-dioxidothiomorpholino)-5-methyl-1H-imidazole-4-carboxamide
2-(4-chloro-2-fluorophenyl)-N-(1,1-dioxidothiomorpholino)-1-(4-(5-hydroxypent-1-yn-1-yl)phenyl)-5-methyl-1H-imidazole-4-carboxamide
2-(4-chloro-2-fluorophenyl)-N-(1,1-dioxidothiomorpholino)-1-(4-(5-hydroxypent-1-yn-1-yl)phenyl)-5-methoxy-1H-imidazole-4-carboxamide
ethyl 6-(4-(2-(4-chloro-2-fluorophenyl)-4-((1,1-dioxidothiomorpholino)carbamoyl)-5-methoxy-1H-imidazol-1-yl)phenyl)hex-5-ynoate
2-(4-chloro-2-fluorophenyl)-1-(4-(5-cyanopent-1-yn-1-yl)phenyl)-N-(1,1-dioxidothiomorpholino)-5-methoxy-1H-imidazole-4-carboxamide
2-(2,4-dichlorophenyl)-N-(1,1-dioxidothiomorpholino)-1-(4-(4-hydroxybut-1-yn-1-yl)phenyl)-5-methyl-1H-imidazole-4-carboxamide
2-(2,4-dichlorophenyl)-N-(1,1-dioxidothiomorpholino)-1-(4-(4-hydroxybut-1-yn-1-yl)phenyl)-5-methoxy-1H-imidazole-4-carboxamide
1-(4-(4-cyanobut-1-yn-1-yl)phenyl)-2-(2,4-dichlorophenyl)-N-(1,1-dioxidothiomorpholino)-5-methoxy-1H-imidazole-4-carboxamide
4-(4-(2-(2,4-dichlorophenyl)-4-((1,1-dioxidothiomorpholino)carbamoyl)-5-methoxy-1H-imidazol-1-yl)phenyl)but-3-yn-1-yl nitrate
1-(4-(5-amino-5-oxopent-1-yn-1-yl)phenyl)-2-(2,4-dichlorophenyl)-N-(1,1-dioxidothiomorpholino)-5-methoxy-1H-imidazole-4-carboxamide
1-(4-(5-amino-5-oxopent-1-yn-1-yl)phenyl)-2-(2,4-dichlorophenyl)-N-(1,1-dioxidothiomorpholino)-5-methyl-1H-imidazole-4-carboxamide
1-(4-(4-cyanobut-1-yn-1-yl)phenyl)-2-(2,4-dichlorophenyl)-N-(1,1-dioxidothiomorpholino-2,2,3,3,5,5,6,6-d8)-5-methyl-1H-imidazole-4-carboxamide
2-(2,4-dichlorophenyl)-N-(1,1-dioxidothiomorpholino-2,2,3,3,5,5,6,6-d8)-1-(4-(4-hydroxybut-1-yn-1-yl)phenyl)-5-methyl-1H-imidazole-4-carboxamide
4-(4-(2-(2,4-dichlorophenyl)-4-((1,1-dioxidothiomorpholino-2,2,3,3,5,5,6,6-d8)carbamoyl)-5-methyl-1H-imidazol-1-yl)phenyl)but-3-yn-1-yl nitrate
4-(4-(2-(2,4-dichlorophenyl)-4-((1,1-dioxidothiomorpholino-2,2,3,3,5,5,6,6-d8)carbamoyl)-5-methoxy-1H-imidazol-1-yl)phenyl)but-3-yn-1-yl nitrate
1-(4-(4-cyanobut-1-yn-1-yl)phenyl)-2-(2,4-dichlorophenyl)-N-(1,1-dioxidothiomorpholino-2,2,3,3,5,5,6,6-d8)-5-methoxy-1H-imidazole-4-carboxamide
2-(2,4-dichlorophenyl)-N-(1,1-dioxidothiomorpholino-2,2,3,3,5,5,6,6-d8)-1-(4-(4-hydroxybut-1-yn-1-yl)phenyl)-5-methoxy-1H-imidazole-4-carboxamide
1-(4-(5-amino-5-oxopent-1-yn-1-yl)phenyl)-2-(2,4-dichlorophenyl)-N-(1,1-dioxidothiomorpholino-2,2,3,3,5,5,6,6-d8)-5-methoxy-1H-imidazole-4-carboxamide
1-(4-(5-amino-5-oxopent-1-yn-1-yl)phenyl)-2-(2,4-dichlorophenyl)-N-(1,1-dioxidothiomorpholino-2,2,3,3,5,5,6,6-d8)-5-methyl-1H-imidazole-4-carboxamide
1-(4-(4-cyanobut-1-yn-1-yl)phenyl)-2-(2,4-dichlorophenyl)-N-(1,1-dioxidothiomorpholino)-5-(methoxymethyl)-1H-imidazole-4-carboxamide
2-(2,4-dichlorophenyl)-N-(1,1-dioxidothiomorpholino)-1-(4-(4-hydroxybut-1-yn-1-yl)phenyl)-5-(methoxymethyl)-1H-imidazole-4-carboxamide
4-(4-(2-(2,4-dichlorophenyl)-4-((1,1-dioxidothiomorpholino)carbamoyl)-5-(methoxymethyl)-1H-imidazol-1-yl)phenyl)but-3-yn-1-yl nitrate
1-(4-(5-amino-5-oxopent-1-yn-1-yl)phenyl)-2-(2,4-dichlorophenyl)-N-(1,1-dioxidothiomorpholino)-5-(methoxymethyl)-1H-imidazole-4-carboxamide
1-(4-(4-cyanobut-1-yn-1-yl)phenyl)-2-(2,4-dichlorophenyl)-N-(1,1-dioxidothiomorpholino)-5-((methoxy-d3)methyl)-1H-imidazole-4-carboxamide
2-(2,4-dichlorophenyl)-N-(1,1-dioxidothiomorpholino)-1-(4-(4-hydroxybut-1-yn-1-yl)phenyl)-5-((methoxy-d3)methyl)-1H-imidazole-4-carboxamide
1-(4-(5-amino-5-oxopent-1-yn-1-yl)phenyl)-2-(2,4-dichlorophenyl)-N-(1,1-dioxidothiomorpholino)-5-((methoxy-d3)methyl)-1H-imidazole-4-carboxamide
4-(4-(2-(2,4-dichlorophenyl)-4-((1,1-dioxidothiomorpholino)carbamoyl)-5-((methoxy-d3)methyl)-1H-imidazol-1-yl)phenyl)but-3-yn-1-yl nitrate
3-((4-(2-(2,4-dichlorophenyl)-4-((1,1-dioxidothiomorpholino)carbamoyl)-5-methoxy-1H-imidazol-1-yl)phenyl)ethynyl)pyridine 1-oxide
3-((4-(2-(2,4-dichlorophenyl)-4-((1,1-dioxidothiomorpholino)carbamoyl)-5-methyl-1H-imidazol-1-yl)phenyl)ethynyl)pyridine 1-oxide
2-(2,4-dichlorophenyl)-N-(1,1-dioxidothiomorpholino)-5-methyl-1-(4-(pyridin-3-ylethynyl)phenyl)-1H-imidazole-4-carboxamide
2-(2,4-dichlorophenyl)-N-(1,1-dioxidothiomorpholino)-5-methoxy-1-(4-(pyridin-3-ylethynyl)phenyl)-1H-imidazole-4-carboxamide
2-(2,4-dichlorophenyl)-N-(1,1-dioxidothiomorpholino-2,2,3,3,5,5,6,6-d8)-5-methyl-1-(4-(pyridin-3-ylethynyl)phenyl)-1H-imidazole-4-carboxamide
2-(2,4-dichlorophenyl)-N-(1,1-dioxidothiomorpholino-2,2,3,3,5,5,6,6-d8)-5-methoxy-1-(4-(pyridin-3-ylethynyl)phenyl)-1H-imidazole-4-carboxamide
tert-butyl 5-(4-(2-(2,4-dichlorophenyl)-4-((1,1-dioxidothiomorpholino)carbamoyl)-5-methyl-1H-imidazol-1-yl)phenyl)pent-4-ynoate
ethyl 5-(4-(2-(2,4-dichlorophenyl)-4-((1,1-dioxidothiomorpholino)carbamoyl)-5-methyl-1H-imidazol-1-yl)phenyl)pent-4-ynoate
1-(4-(5-cyanopent-1-yn-1-yl)phenyl)-2-(2,4-dichlorophenyl)-N-(1,1-dioxidothiomorpholino)-5-methyl-1H-imidazole-4-carboxamide
5-(4-(2-(2,4-dichlorophenyl)-4-((1,1-dioxidothiomorpholino)carbamoyl)-5-methyl-1H-imidazol-1-yl)phenyl)pent-4-yn-1-yl nitrate
5-(4-[4-cyanobut-1-ynyl]phenyl)-1-(2,4-dichlorophenyl)-4-methyl-N-(1,1-dioxo-thiomorpholino)-1H-pyrazole-3-carboxamide
1-(2-chloro-4-fluorophenyl)-5-(4-(4-cyanobut-1-yn-1-yl)phenyl)-N-(1,1-dioxidothiomorpholino)-4-ethyl-1H-pyrazole-3-carboxamide;
5-(4-chlorophenyl)-1-(2,4-dichlorophenyl)-N-(1,1-dioxidothiomorpholino)-4-methyl-1H-pyrazole-3-carboxamide;
5-(4-bromophenyl)-1-(2,4-dichlorophenyl)-N-(1,1-dioxidothiomorpholino)-4-methyl-1H-pyrazole-3-carboxamide;
1-(2,4-dichlorophenyl)-N-(1,1-dioxidothiomorpholino)-5-(4-iodophenyl)-4-methyl-1H-pyrazole-3-carboxamide;
5-(4-chlorophenyl)-1-(2,4-dichlorophenyl)-N-(1,1-dioxidothiomorpholino)-4-ethyl-1H-pyrazole-3-carboxamide;
5-(4-bromophenyl)-1-(2,4-dichlorophenyl)-N-(1,1-dioxidothiomorpholino)-4-ethyl-1H-pyrazole-3-carboxamide;
1-(2,4-dichlorophenyl)-N-(1,1-dioxidothiomorpholino)-4-ethyl-5-(4-iodophenyl)-1H-pyrazole-3-carboxamide;
1-(2-chloro-4-fluorophenyl)-5-(4-chlorophenyl)-N-(1,1-dioxidothiomorpholino)-4-ethyl-1H-pyrazole-3-carboxamide;
5-(4-bromophenyl)-1-(2-chloro-4-fluorophenyl)-N-(1,1-dioxidothiomorpholino)-4-ethyl-1H-pyrazole-3-carboxamide;
1-(2-chloro-4-fluorophenyl)-N-(1,1-dioxidothiomorpholino)-4-ethyl-5-(4-iodophenyl)-1H-pyrazole-3-carboxamide; and
4-(4-(1-(2,4-dichlorophenyl)-3-((1,1-dioxidothiomorpholino)carbamoyl)-4-methyl-1H-pyrazol-5-yl)phenyl)but-3-yn-1-yl nitrate.
5-(4-(5-(2,4-dichlorophenyl)-2-((1,1-dioxidothiomorpholino)amino)thiazol-4-yl)phenyl)pent-4-ynenitrile
4-((5-(2,4-dichlorophenyl)-4-(4-(4-hydroxybut-1-yn-1-yl)phenyl)thiazol-2-yl)amino)thiomorpholine 1,1-dioxide
4-(4-(5-(2,4-dichlorophenyl)-2-((1,1-dioxidothiomorpholino)amino)thiazol-4-yl)phenyl)but-3-yn-1-yl nitrate
5-(4-(5-(2,4-dichlorophenyl)-2-((1,1-dioxidothiomorpholino)amino)oxazol-4-yl)phenyl)pent-4-ynenitrile
4-((5-(2,4-dichlorophenyl)-4-(4-(4-hydroxybut-1-yn-1-yl)phenyl)oxazol-2-yl)amino)thiomorpholine 1,1-dioxide
4-(4-(5-(2,4-dichlorophenyl)-2-((1,1-dioxidothiomorpholino)amino)oxazol-4-yl)phenyl)but-3-yn-1-yl nitrate
4-((5-(2,4-dichlorophenyl)-4-(4-(4-isothiocyanatobut-1-yn-1-yl)phenyl)oxazol-2-yl)amino)thiomorpholine 1,1-dioxide
4-((5-(2,4-dichlorophenyl)-4-(4-(4-isothiocyanatobut-1-yn-1-yl)phenyl)thiazol-2-yl)amino)thiomorpholine 1,1-dioxide
4-((1-(2,4-dichlorophenyl)-5-(4-(4-isothiocyanatobut-1-yn-1-yl)phenyl)-1H-1,2,4-triazol-3-yl)amino)thiomorpholine 1,1-dioxide
4-((1-(2,4-dichlorophenyl)-5-(4-(4-hydroxybut-1-yn-1-yl)phenyl)-1H-1,2,4-triazol-3-yl)amino)thiomorpholine 1,1-dioxide
4-(4-(1-(2,4-dichlorophenyl)-3-((1,1-dioxidothiomorpholino)amino)-1H-1,2,4-triazol-5-yl)phenyl)but-3-yn-1-yl nitrate
5-(4-(1-(2,4-dichlorophenyl)-3-((1,1-dioxidothiomorpholino)amino)-1H-1,2,4-triazol-5-yl)phenyl)pent-4-ynenitrile
5-(4-(4-cyanobut-1-yn-1-yl)phenyl)-1-(2,4-dichlorophenyl)-N-(1,1-dioxidothiomorpholino)-4-methyl-4,5-dihydro-1H-pyrazole-3-carboxamide
5-(4-(4-cyanobut-1-yn-1-yl)phenyl)-1-(2,4-dichlorophenyl)-N-(1,1-dioxidothiomorpholino)-4,5-dihydro-1H-pyrazole-3-carboxamide
1-(2,4-dichlorophenyl)-N-(1,1-dioxidothiomorpholino)-5-(4-(4-hydroxybut-1-yn-1-yl)phenyl)-4,5-dihydro-1H-pyrazole-3-carboxamide
4-(4-(1-(2,4-dichlorophenyl)-3-((1,1-dioxidothiomorpholino)carbamoyl)-4,5-dihydro-1H-pyrazol-5-yl)phenyl)but-3-yn-1-yl nitrate
1-(2,4-dichlorophenyl)-N-(1,1-dioxidothiomorpholino)-5-(4-(4-isothiocyanatobut-1-yn-1-yl)phenyl)-4,5-dihydro-1H-pyrazole-3-carboxamide
(R)-5-(4-(4-cyanobut-1-yn-1-yl)phenyl)-1-(2,4-dichlorophenyl)-N-(1,1-dioxidothiomorpholino)-4,5-dihydro-1H-pyrazole-3-carboxamide
(S)-5-(4-(4-cyanobut-1-yn-1-yl)phenyl)-1-(2,4-dichlorophenyl)-N-(1,1-dioxidothiomorpholino)-4,5-dihydro-1H-pyrazole-3-carboxamide
(R)-1-(2,4-dichlorophenyl)-N-(1,1-dioxidothiomorpholino)-5-(4-(4-hydroxybut-1-yn-1-yl)phenyl)-4,5-dihydro-1H-pyrazole-3-carboxamide
(S)-1-(2,4-dichlorophenyl)-N-(1,1-dioxidothiomorpholino)-5-(4-(4-hydroxybut-1-yn-1-yl)phenyl)-4,5-dihydro-1H-pyrazole-3-carboxamide
(R)-1-(2,4-dichlorophenyl)-N-(1,1-dioxidothiomorpholino)-5-(4-(4-isothiocyanatobut-1-yn-1-yl)phenyl)-4,5-dihydro-1H-pyrazole-3-carboxamide
(S)-1-(2,4-dichlorophenyl)-N-(1,1-dioxidothiomorpholino)-5-(4-(4-isothiocyanatobut-1-yn-1-yl)phenyl)-4,5-dihydro-1H-pyrazole-3-carboxamide

In a preferred embodiment, a compound of claim 1 can preferentially acts on one or more of the following model systems or pathways, without acting on certain others, to treat a human disease or condition.

CB 1 Cannabinoid 1 receptor

CB2 Cannabinoid 2 receptor

Monocyte chemoattractant pratein-1 {MCP-1/CCL2) is a chemoattractant cytokine (chemokine) that mediates recruitment of monocytes and T cells into sites of inflammation. MCP-1 is categorized as an inflammation-related activity in the 3C system modeling Th1 vascular inflammation. MCP-1 in the 3C system is regulated by the following pathways: HDAC. Histamine H1 R. IKK2.

Vascular Cell Adhesion Molecule 1 (VCAM-1/CD106) is a cell adhesion molecule that mediates adhesion of monocytes and T cells to endothelial cells. VCAM-1 is categorized as an inflammation-related activity in the 3C system modeling Th1 vascular inflammation. VCAM-1 in the 3C system is regulated by the following pathways: HDAC. Histamine H1 R. IKK2.

Thrombomodulin/CD141 is a cell surface receptor for complement factor 3b with anti-coagulant anti-inflammatory and cytoprotective activities during the process of fibrinolysis. coagulation and thrombosis. Thrombomodulin is categorized as a hemostasis-related activity in the 3C system modeling Th1 vascular inflammation.

Tissue Factor/CD142 is a cell surface receptor for coagulation factor VII that promotes the format ion of thrombin during the process of vascular thrombosis and coagulation. Tissue Factor is categorized as a hemostasis-related activity in the 3C system modeling Th1 vascular inflammation. TF in the 3C system is regulated by the following pathways: HDAC. IKK2.

Intercellular Adhesion Molecule 1 (ICAM-1/CD54) is a cell adhesion molecule that mediates leukocyte-endothelial cell adhesion and leukocyte recruitment.

ICAM-1 is categorized as an inflammation-related activity in the 3C system modeling Th1 vascular inflammation. ICAM-1 in the 3C system is regulated by the following pathways: IKK2.

E-Selectin/CD62E is a cell adhesion molecule expressed only on endothelial cells that mediates leukocyte-endothelial cell interactions. E-Selectin is categorized as an inflammation-related activity in the 3C system modeling Th1 vascular inflammation. E-selectin in the 3C system is regulated by the following pathways: IKK2.

Urokinase plasminogen activator receptor {uPAR/CD87) is a cell surface receptor for urokinase plasminogen activator (LIPA) involved in the regulation of pericellular proteolysis. cell migration, cancer cell invasion, and angiogenesis. uPAR is categorized as a t issue remodeling-related activity in the 3C system modeling Th1 vascular inflammation. uPAR in the 3C system is regulated by the following pathways: HDAC, HMG-CoA Reductase, Histamine H1 R Microtubule, P13K. mTOR.

Interleukin-8 {IL-8/CXCLB) is a chemokine that mediates neutrophil recruitment into acute inflammatory sites. IL-8 is categorized as an inflammation-related activity in the 3C system modeling Th1 vascular inflammation. IL-8 in the 3C system is regulated by the following pathways: IKK2, p38 MAPK.

Monokine induced by gamma interferon (MIG/CXCL9) is a chemokine that mediates T cell recruitment. MIG is categorized as an inflammation-related activity in the 3C system modeling Th1 vascular inflammation. MIG in the 3C system is regulated by the following pathways: HDAC. Histamine H1 R. IKK2, JAK.

HLA-DR is a cell surface heterodimer involved in antigen presentation. HLA-DR binds peptides and presents them to the T cell receptor and is involved in T cell activation and immune responses. HLA-DR is categorized as an immunomodulatory-related activity in the 3C system modeling Th1 vascular inflammation. HLADR in the 3C system is regulated by the following pathways: HDAC. Histamine H 1 R. IKK2. JAK. P13K. RAR/RXR. p38 MAPK.

Proliferation in the 3C system is a measure of endothelial cell proliferation which is important to the process of wound healing and angiogenesis. Proliferation in the 3C system is regulated by the following pathways: EGFR. HDAC, HMG-CoA Reductase. Histamine H1 R. IKK2, MEK. Microtubule, P13K. Src, mTOR.

SRB in the 3C system is a measure of the total protein content of venular endothelial cells. Cell viability of adherent cells is measured by Sulforhodamine B {SRB} staining, a method that determines cell density by measuring total protein content of test wells. SRB in the 3C system is regulated by the following pathways: HMG-CoA Reductase, Histamine H1 R Microtubule, PI3K.

Monocyte chemoattractant protein-1 (MCP-1/CCL2) is a chemoattractant cytokine {chemokine) that regulates the recruitment of monocytes and T cells into sites of inflammation. MCP-1 is categorized as an inflammation-related activity in the 4H system modeling Th2 vascular inflammation. MCP-1 in the 4H system is regulated by the following pathways: HMG-CoA Reductase, Histamine H1 R.

Eotaxin-3/CCL26 is a chemokine that mediates recruitment of eosinophils and basophils into sites of t issue inflammation. Eotaxin-3 is categorized as an inflammation-related activity in the 4H system modeling Th2 vascular inflammation. Eotaxin 3 in the 4H system is regulated by the following pathways: HDAC Histamine H1 R, IKK2, JAK. RAR/RXR.

Vascular Cell Adhesion Molecule 1 (VCAM-1/CD106) is a cell adhesion molecule that mediates adhesion of monocytes and T cells to endothelial cells. VCAM-1 is categorized as an inflammation-related activity in the 4H system modeling Th2 vascular inflammation. VCAM-1 in the 4H system is regulated by the following pathways: Calcineurin. HDAC. Histamine H1 R. IKK2, JAK. RAR/RXR.

P-Selectin/CD62P is a cell adhesion molecule that mediates platelet-endothelial cell and leukocyte-endothelial cell interactions. P-Selectin is categorized as an inflammation-related activity in the 4H system modeling Th2 vascular inflammation. P-selectin in the 4H system is regulated by the following pathways: JAK P13K.

Urokinase plasminogen activator receptor {uPAR/CD87) is a cell surface receptor for urokinase plasminogen activator (uPA) involved in the regulation of pericellular proteolysis, cell migration, cancer cell invasion, and angiogenesis. uPAR is categorized as a tissue remodeling-related activity in the 4H system modeling Th2 vascular inflammation. uPAR in the 4H system is regulated by the following pathways: EGFR HDAC Histamine H1 R MEK Microtubule.

SRB in the 4H system is a measure of the total protein content of venular endothelial cells. Cell viability of adherent cells is measured by Sulforhodamine B {SRB} staining, a method that determines cell density by measuring total protein content of test wells. SRB in the 4H system is regulated by the following pathways: Histamine H1 R.

Vascular endothelial growth factor receptor 2 (VEGFR2) in the 4H system modeling Th2 vascular inflammation is a cell surface tyrosine kinase receptor for VEGFA and is involved in angiogenesis, endothelial cell proliferation and vascular permeability.

Monocyte chemoattractant protein-1 (MCP-1/CCL2) is a chemoattractant cytokine (chemokine) that regulates the recruitment of monocytes and T cells into sites of inflammation. MCP-1 is categorized as an inflammation-related activity in the LPS system modeling monocyte-driven Th1 vascular inflammation. MCP-1 in the LPS system is regulated by the following pathways: HDAC. Histamine H1 R. IKK2, p38 MAPK.

Vascular Cell Adhesion Molecule 1 (VCAM-1/CD106) is a cell adhesion molecule that mediates adhesion of monocytes and T cells to endothelial cells. VCAM-1 is categorized as an inflammation-related activity in the LPS system modeling monocyte-driven Th1 vascular inflammation. VCAM-1 in the LPS system is regulated by the following pathways: HDAC. IKK2.

Thrombomodulin/CD141 is a cell surface receptor for complement factor 3b with anti-coagulant anti-inflammatory and cytoprotective activities during the process of fibrinolysis, coagulation and thrombosis. Thrombomodulin is categorized as a hemostasis-related activity in the LPS system modeling monocyte driven Th1 vascular inflammation. TM in the LPS system is regulated by the following pathways: IKK2, RAR/RXR. p38 MAPK.

Tissue Factor/CD142 is a cell surface receptor for coagulation factor VII that promotes the formation of thrombin during the process of thrombosis and coagulation. Tissue Factor is categorized as a hemostasis-related activity in the LPS system modeling monocyte-driven Th1 vascular inflammation. TF in the LPS system is regulated by the following pathways: IKK2. p38 MAPK.

CO40 is a cell surface adhesion receptor and costimulatory receptor for T cell activation that is expressed on antigen presenting cells, endothelial cells. Smooth muscle cells, fibroblasts and epithelial cells. CD40 is categorized as an immunomodulatory-related activity in the LPS system modeling monocyte-driven Th1 vascular inflammation. CD40 in the LPS system is regulated by the following pathways: Histamine H1 R IKK2, PI3K. RAR/RXR Src, mTOR.

E-Selectin/CD62E is a cell adhesion molecule expressed only on endothelial cells that mediates leukocyte-endothelial cell interactions. E-Selectin is categorized as an inflammation-related activity in the LPS system modeling monocyte-driven Th1 vascular inflammation. E-selectin in the LPS system is regulated by the following pathways: HDAC. IKK2. p38 MAPK.

CD69 is a cell surface activation antigen. CD69 is categorized as an immunomodulatory-related activity in the LPS system modeling monocyte-driven Th1 vascular inflammation. CD69 in the LPS system is regulated by the following pathways: Histamine H1 R IKK2.

Interleukin-8 {IL-8/CXCL8) is a chemokine that mediates neutrophil recruitment into acute inflammatory sites. IL-8 is categorized as an inflammation-related activity in the LPS system modeling monocyte-driven Th1 vascular inflammation. IL-8 in the LPS system is regulated by the following pathways: IKK2, p38 MAPK.

Interleukin-1 alpha {IL-1a) is a secreted proinflammatory cytokine involved in endothelial cell activation and neutrophil recruitment. IL-1a is categorized as an inflammation-related activity in the LPS system modeling monocyte-driven Th1 vascular inflammation. IL-1a in the LPS system is regulated by the following pathways: HDAC, HMG-CoA Reductase. IKK2. p38 MAPK.

Macrophage colony-stimulating factor (M-CSF) is a secreted and cell surface cytokine that mediates macrophage differentiation. M-CSF is categorized as a tissue remodeling-related activity in the LPS system modeling monocyte-driven Th1 vascular inflammation. M-CSF in the LPS system is regulated by the following pathways: HDAC. IKK2. RAR/RXR, p38 MAPK.

Prostaglandin E2 {PGE2) is an immunomodulatory lipid mediator involved in muscle contractility, inflammatory pain and kidney function. Secreted PGE2 (sPGE2) is categorized as an inflammation-related activity in the LPS system modeling monocyte-driven Th1 vascular inflammation. sPGE2 in the LPS system is regulated by the following pathways: IKK2. MEI<. PKC (c+n). RAR/RXR. Vitamin DR. mTOR. p38 MAPK.

SRB in the LPS system is a measure of the total protein content of venular endothelial cells and PBMC. Cell viability of adherent cells is measured by Sulforhodamine B (SRB) staining. a method that determines cell density by measuring total protein content of test wells. SRB in the LPS system is regulated by the following pathways: HMG-CoA Reductase, Histamine H1 R IKK2.

Tumor necrosis factor alpha {TNFa) is a secreted proinflammatory cytokine involved in Th1 vascular inflammation. Secreted TNFa (sTNFa) is categorized as an inflammation-related activity in the LPS system modeling monocyte-driven Th1 vascular inflammation. sTNFa in the LPS system is regulated by the following pathways: EGFR, Glucocorticoid R. HDAC. Histamine H1 R. IKK2. MEK. PDE4, P13K. PKC (c+n). RAR/RXR, Src. TNF-alpha. Vitamin DR. p38 MAPK.

Monocyte chemoattractant protein-1 (MCP-1/CCL2) is a chemoattractant cytokine (chemokine) that regulates the recruitment of monocytes and T cells into sites of inflammation. MCP-1 is categorized as an inflammation-related activity in the SAg system modeling T cell-driven Th1 vascular inflammation. MCP-1 in the SAg system is regulated by the following pathways: Calcineurin. HDAC. IKK2. JAK. ME<. P13K. PKC (c+n). Src.

CD38 is a cell surface enzyme and marker of cell activation that is involved in T cell activation/co-stimulation and chemotaxis. CD38 is categorized as an immunomodulatory-related activity in the SAg system modeling T cell-d riven Th1 vascular inflammation. CD38 in the SAg system is regulated by the following pathways: Calcineur in. HDAC. IKK2. JAK. MEI<. P13K. PKC (c+n). RAR/RXR. Src.

CD40 is a cell surface adhesion receptor and costimulatory receptor for T cell activation that is expressed on antigen presenting cells. endothelial cells, smooth muscle cells, fibroblasts and epithelial cells. CD40 is categorized as an immunomodulatory-related activity in the SAg system modeling T cell-driven Th1 vascular inflammation. CD40 in the SAg system is regulated by the following pathways: Calcineurin, HMG-CoA Reductase, Histamine H1 R IKK2, JAK. PI3K RAR/RXR, Sr c. mTOR.

E-Selectin/CD62E is a cell adhesion molecule expressed only on endothelial cells that mediates leukocyte-endothelial cell interactions. E-Selectin is categorized as an inflammation-related activity in the SAg system modeling T cell-driven Th1 vascular inflammation. E-selectin in the SAg system is regulated by the following pathways: Calcineurin. Glucocorticoid R. HMG-CoA Reductase. Histamine H1 R. IKK2. JAK. MEI<. PDE4, P13K. PKC (c+n). RAR/RXR. Src. TNF-alpha.

CD69 is a cell surface activation antigen that is induced early during immune activation and is involved in lymphocyte proliferation and activation. CD69 is categorized as an immunomodulatory-related activity in the SAg system modeling T cell-driven Th1 vascular inflammation. CD69 in the SAg system is regulated by the following pathways: Calcineurin. EGFR. HMG-CoA Reductase. Histamine H1 R. IKK2. JAi<. MEI<. PKC (c+n). Src.

Interleukin-a (IL-8/CXCL8) is a chemokine that mediates neutrophil recruitment into acute inflammatory sites. IL-8 is categorized as an inflammation-related activity in the SAg system modeling T cell-driven Th1 vascular inflammation. IL-8 in the SAg system is regulated by the following pathways: Calcineurin. HMGCoA Reductase. IKK2. JAK. ME<. PKC (c+n). Src. TNF-alpha. p38 MAPK.

Monokine induced by gamma interferon (MIG/CXCL9) is a chemokine that mediates T cell recruitment. MIG is categorized as an inflammation-related activity in the SAg system modeling T cell-driven Th1 vascular inflammation. MIG in the SAg system is regulated by the following pathways: Calcineurin, HDAC. IKK2, JAK. RAR/RXR, Sr c.

PBMC Cytotoxicity in the SAg system is a measure of the cell death of PBMC. Cell viability of non-adherent cells is measured by alamarBlueA® staining. A method based on a cell permeable compound that emits fluorescence after entering cells. The number of living cells is proportional to the amount of fluorescence produced. Pcyto in the SAg system is regulated by the following pathways: Histamine H1 R IKK2.

Proliferation in the SAg system is a measure of T cell proliferation which is the critical event driving both adaptive immunity as well as many auto-immune diseases (RA PsA MS, IBD etc). Prolif in the SAg system is regulated by the following pathways: Calcineurin, EGFR HDAC HMG-CoA Reductase, Histamine H1 R IKK2. JAK. MEI<. Microtubule. P13K. PKC (c+n). RAR/RXR. Sr c. mTOR.

SRB in the SAg system is a measure of the total protein content of venular endothelial cells. Cell viability of adherent cells is measured by Sulforhodamine B (SRB) staining a method that determines cell density by measuring total protein content of test wells. SRB in the SAg system is regulated by the following pathways: HMG-CoA Reductase, Histamine H1 R.

B cell proliferation is a critical event driving both adaptive immunity (antibody production) as well as auto-immune diseases where B cells are key disease players (Lupus, MS, RA etc). Inhibition of B cell proliferation is considered an immune suppressive effect. Prolif in the BT system is regulated by the following pathways: Calcineur in. EGFR. HDAC. HMG-CoA Reductase. Histamine H 1 R. IKK2. JAK. Microtubule. P13K. PKC (c+n). Sr c. mTOR.

PBMC Cytotoxicity in the BT system is a measure of the cell death of PBMC. Cell viability of non-adherent cells is measured by alamarBlueA® staining. a method based on a cell permeable compound that emits fluorescence after entering cells. The number of living cells is proportional to the amount of fluorescence produced. Pcyto in the BT system is regulated by the following pathways: Calcineurin. Glucocorticoid R. HDAC. Histamine H1 R. IKK2. P13K.

Secreted IgG {sIgG) is produced by B cells and is the main type of antibody found in blood and extracellular fluid that mediates the immune response against pathogens. sIgG is categorized as an immunomodulatory-related activity in the BT system modeling T cell dependent B cell activation. sIgG in the BT system is regulated by the following pathways: Calcineur in. EGFR. HDAC. HMG-CoA Reductase. Histamine H1 R. IKK2. JAK. Microtubule. PDE4. P13K. PKC (c+n). Src. Vitamin D R. mTOR. p38 MAPK.

Interleukin-17A {Il-17A) is a proinflammatory cytokine produced by T cells that induces cytokine production and mediates monocyte and neutrophil recruitment to sites of inflammation. secreted Il-17A {s-17A) is categorized as an immunomodulatory-related activity in the BT system modeling T cell dependent B cell activation. sIl-17A in the BT system is regulated by the following pathways: Calcineurin, EGFR Glucocorticoid R HDAC HMG-CoA Reductase, Histamine H1 R IKK2. JAK. MEK. Microtubule. P13K. PKC (c+n). Src. Vitamin DR. mTOR, p38 MAPK.

Interleukin-17F {IL-17F) is a proinflammatory cytokine produced by T cells that induces cytokine, chemokine and adhesion molecule production and mediates neutrophil recruitment to sites of inflammation. Secreted IL-17F (s Il-17F) is categorized as an immunomodulatory-related activity in the BT system modeling T cell dependent B cell activation. sIL-17F in the BT system is regulated by the following pathways: Calcineurin. EGFR. Glucocorticoid R. HDAC. HMG-CoA

Reductase. Histamine H1 R. JAK MEK Microtubule. PKC (c+n). RAR/RXR. Src. TNF-alpha. Vitamin DR. mTOR. p38 MAPK.

Interleukin-2 (IL-2) is a secreted proinflammatory cytokine produced by T cells that regulates lymphocyte proliferation and promotes T cell differentiation.

Secreted IL-2 (IL-2) is categorized as an immunomodulatory-related activity in the BT system modeling T cell dependent B cell activation. sIL-2 in the BT system is regulated by the following pathways: Calcineurin. EGFR. Glucocorticoid R. HDAC. Histamine H1 R. IKK2. JAK MEK P13K PKC (c+n). RAR/RXR. Src. Vitamin D R. mTOR. p38 MAPK.

Interleukin-6 (IL-6) is a secreted proinflammatory cytokine and acute phase reactant. Secreted IL-6 (s IL-6) is categorized as an immunomodulatory-related activity in the BT system modeling T cell dependent B cell activation. sIL-6 in the BT system is regulated by the following pathways: Calcineurin, EGFR. Glucocorticoid R. HDAC. Histamine H1 R. IKK2, JAK MEK P13K PKC (c+n), Src. TNF-alpha, Vitamin D R. mTOR. p38 MAPK.

Tumor necrosis factor alpha (TNFa) is a secreted proinflammatory cytokine involved in Th1 inflammation. Secreted TNFa {sTNFa) is categorized as an inflammation-related activity in the BT system modeling T cell dependent B cell activation. sTNFa in the BT system is regulated by the following pathways: Calcineurin. EGFR. Glucocorticoid R. HDAC. HMG-CoA Reductase. Histamine H1 R. IKK2, JAK MEK P13K PKC (c+n). RAR/RXR. Src. TNF-alpha. Vitamin D R. mTOR.

Monocyte chemoattractant protein-1 (MCP-1/CCL2) is a chemoattractant cytokine (chemokine) that regulates the recruitment of monocytes and T cells into sites of inflammation. MCP-1 is categorized as an inflammation-related activity in the BF4T system modeling Th2 airway inflammation.

Eotaxin-3/CCL26 is a chemokine that mediates recruitment of eosinophils and basophils into t issue sites. Eotaxin-3 is categorized as an inflammation-related activity in the BF4T system modeling Th2 airway inflammation. Eotaxin 3 in the BF4T system is regulated by the following pathways: EGFR. RAR/RXR.

Vascular Cell Adhesion Molecule 1 {VCAM-1/CD106} is a cell adhesion molecule that mediates adhesion of monocytes and T cells to endothelial cells. VCAM-1 is categorized as an inflammation-related activity in the BF4T system modeling Th2 airway inflammation.

Intercellular Adhesion Molecule 1 (ICAM-1/CD54) is a cell adhesion molecule that mediates leukocyte-endothelial cell adhesion and leukocyte recruitment.

ICAM-1 is categorized as an inflammation-related activity in the BF4T system modeling Th2 airway inflammation.

CD90 is a cell surface glycoprotein that mediates cell-cell and cell-matrix interactions. CD90 is categorized as a tissue remodeling-related activity in the BF4T system modeling Th2 airway inflammation.

Interleukin-8 (Il-8/CXCL8) is a chemokine that mediates neutrophil recruitment into acute inflammatory sites. Il-8 is categorized as an inflammation-related activity in the BF4T system modeling Th2 airway inflammation. IL-8 in the BF4T system is regulated by the following pathways: IKK2.

Interleukin-1 alpha {Il-1a} is a secreted proinflammatory cytokine involved in endothelial cell activation and neutrophil recruitment. Secreted Il-1a (s Il-1a) is categorized as an inflammation-related activity in the BF4T system modeling Th2 airway inflammation.

Keratin 8/18 is an intermediate filament heterodimer of fibrous structural proteins involved in Epithelial cell death. EMT. COPD. Lung Inflammation. Keratin 8/18 is categorized as a tissue remodeling-related activity in the BF4T system modeling Th2 airway inflammation.

Matrix metalloproteinase-1 (MMP-1) is an interstitial collagenase that degrades collagens I. II and III and is involved in the process of tissue remodeling. MMP-1 is categorized as a tissue remodeling-related activity in the BF4T system modeling Th2 airway inflammation.

Matrix metalloproteinase-3 (MMP-3) is an enzyme involved in tissue remodeling that can activate other MMPs (MMP-1, MMP-7 and MMP-9) and degrade collagens (II, III, IV, IX and X}, proteoglycans, fibronectin, laminin and elastin. MMP-3 is categorized as a tissue remodeling-related activity in the BF4T system modeling Th2 airway inflammation.

Matrix metalloproteinase-9 (MMP-9) is a gelatinase B that degrades collagen IV and gelatin and is involved in airway matrix remodeling. MMP-9 is categorized as a tissue remodeling-related activity in the BF4T system modeling Th2 airway inflammation.

Plasminogen activator inhibitor-1 {PAI-I) is a serine proteinase inhibitor and inhibitor of tissue plasminogen activator (tPA) and urokinase (LIPA) and is involved in tissue remodeling and fibrinolysis. PAI-I is categorized as a t issue remodeling-related activity in the BF4T system modeling Th2 airway inflammation.

SRB in the BF4T system is a measure of the total protein content of bronchial epithelial cells and dermal fibroblasts. Cell viability of adherent cells is measured by Sulforhodamine B (SRB) staining. a method that determines cell density by measuring total protein content of test wells.

Tissue plasminogen activator (tPA) is a serine protease that catalyzes the cleavage of plasminogen to plasmin and regulates clot degradation. tPA is involved in fibrinolysis, cell migration and tissue remodeling. tPA is categorized as a tissue remodeling-related activity in the BF4T system modeling Th2 airway inflammation.

Urokinase plasminogen activator (uPA) is a serine protease with thrombolytic activity. Triggers fibrinolysis and extracellular matrix degradation. uPA is categorized as a tissue remodeling-related activity in the BF4T system modeling Th2 airway inflammation.

Intercellular Adhesion Molecule 1 (ICAM-1/CD54) is a cell adhesion molecule that mediates leukocyte-endothelial cell adhesion and leukocyte recruitment.

ICAM-1 is categorized as an inflammation-related activity in the BE3C system modeling Th1 lung inflammation.

Urokinase plasminogen activator receptor {uPAR/CD87) is a cell surface receptor for urokinase plasminogen activator (uPA) involved in the regulation of pericellular proteolysis. cell migration. cancer cell invasion. and angiogenesis. uPAR is categorized as a tissue remodeling-related activity in the BE3C system modeling Th1 lung inflammation. uPAR in the BE3C system is regulated by the following pathways: EGFR. HDAC. IKK2. JAK Src.

Interferon-gamma-inducible protein 10 (IP-10/CXCL10) is a chemokine that mediates T cell. monocyte and dendritic cell chemotaxis. IP-10 is categorized as an inflammation-related activity in the BE3C system modeling Th1 lung inflammation. IP-10 in the BE3C system is regulated by the following pathways: IKK2. JAK RAR/RXR.

Interferon-inducible T Cell Alpha Chemoattractant (I-TAC/CXCL1 1) is a chemokine that mediates T cell and monocyte chemotaxis. 1-TAC is categorized as an inflammation-related activity in the BE3C system modeling Th1 lung inflammation. I-TAC in the BE3C system is regulated by the following pathways: HDAC. IKK2. JAK.

Interleukin-8 (Il-8/CXCL8) is a chemokine that mediates neutrophil recruitment into acute inflammatory sites. IL-8 is categorized as an inflammation-related activity in the BE3C system modeling Th1 lung inflammation. IL-8 in the BE3C system is regulated by the following pathways: IKK2.

Monokine induced by gamma interferon (MIG/CXCL9) is a chemokine that mediates T cell recruitment. MIG is categorized as an inflammation-related activity in the BE3C system modeling Th1 lung inflammation. MIG in the BE3C system is regulated by the following pathways: JAK.

Epidermal growth factor receptor (EGFR) is a cell surface receptor for epidermal growth factor involved in cell proliferation, cell differentiation, tissue remodeling and tumor growth. EGFR is categorized as a tissue remodeling-related activity in the BE3C system modeling Th1 lung inflammation.

HLA-OR is a cell surface heterodimer involved in antigen presentation. HLA-OR binds and presents peptides to T cell receptors and is involved in T cell activation and immune responses. HLA-DR is categorized as an immunomodulatory-related activity in the BE3C system modeling Th1 lung inflammation. HLADR in the BE3C system is regulated by the following pathways: JAK.

Interleukin-1 alpha (Il-1 a} is a secreted proinflammatory cytokine involved in endothelial cell activation and neutrophil recruitment. secreted Il-1 a (s Il-1 a) is categorized as an inflammation-related activity in the BE3C system modeling Th1 lung inflammation. IL-1a in the BE3C system is regulated by the following pathways: EGFR. Src.

Keratin 8/18 is an intermediate filament heterodimer of fibrous structural proteins involved in Epithelial cell death, EMT, COPD. Lung Inflammation. Keratin 8/18 is categorized as a tissue remodeling-related activity in the BE3C system modeling Th1 lung inflammation.

Matrix metalloproteinase-1 {MMP-1) is an interstitial collagenase that degrades collagens I, II and III and is involved in the process of tissue remodeling. MMP-1 is categorized as a tissue remodeling-related activity in the BE3C system modeling Th1 lung inflammation. MMP-1 in the BE3C system is regulated by the following pathways: EGFR. MEK. Src.

Matrix metalloproteinase-9 (MMP-9) is a gelatinase B that degrades collagen IV and gelatin and is involved in airway matrix remodeling. MMP-9 is categorized as a tissue remodeling-related activity in the BE3C system modeling Th1 lung inflammation. MMP-9 in the BE3C system is regulated by the following pathways: EGFR. IKK2, Microtubule, PI3K.

Plasminogen activator inhibitor-1 (PAI-I} is a serine proteinase inhibitor and inhibitor of tissue plasminogen activator (tPA) and urokinase (uPA) and is involved in tissue remodeling and fibrinolysis. PAI-I is categorized as a t issue remodeling-related activity in the BE3C system modeling Th1 lung inflammation. PAI-1 in the BE3C system is regulated by the following pathways: EGFR. MEK. Src.

SRB in the BE3C system is a measure of the total protein content of bronchial epithelial cells. Cell viability of adherent cells is measured by Sulforhodamine B {SRB) staining. a method that determines cell density by measuring total protein content of test wells.

Tissue plasminogen activator (tPA) is a serine protease that catalyzes the cleavage of plasminogen to plasmin and regulates clot degradation. tPA is involved in cell migration, t issue remodeling and fibrinolysis. tPA is categorized as a tissue remodeling-related activity in the BE3C system modeling Th1 lung inflammation. tPA in the BE3C system is regulated by the following pathways: EGFR. HDAC. HMG-CoA Reductase.

Urokinase plasminogen activator {uPA) is a serine protease with thrombolytic activity. Triggers fibrinolysis and extracellular matrix degradation. uPA is categorized as a tissue remodeling-related activity in the BE3C system modeling Th1 lung inflammation. uPA in the BE3C system is regulated by the following pathways: EGFR. HDAC, IKK2. JAK. Src.

Monocyte chemoattractant protein-1 (MCP-1/CCL2) is a chemoattractant cytokine {chemokine) that regulates the recruitment of monocytes and T cells into sites of inflammation. MCP-1 is categorized as an inflammation-related activity in the CASM3C system modeling Th1 vascular smooth muscle inflammation. MCP-1 in the CASM3C system is regulated by the following pathways: Glucocorticoid R. HDAC. IKK2.

Vascular Cell Adhesion Molecule 1 (VCAM-1/CD106) is a cell adhesion molecule that mediates adhesion of monocytes and T cells to endothelial cells. VCAM-1 is categorized as an inflammation-related activity in the CASM3C system modeling Th1 vascular smooth muscle inflammation. VCAM-1 in the CASM3C system is regulated by the following pathways: EGFR. HDAC. JAK.

Thrombomodulin/CD141 is a cell surface receptor for complement factor 3b with anti-coagulant anti-inflammatory and cytoprotective activities during the process of fibrinolysis, coagulation, and thrombosis. Thrombomodulin is categorized as a hemostasis-related activity in the CASM3C system modeling ThI vascular smooth muscle inflammation. TM in the CASM3C system is regulated by the following pathways: Glucocorticoid R HMG-CoA Reductase, IKK2, RAR/RXR Vitamin DR. Tissue Factor/CD142 is a cell surface receptor for coagulation factor VII that promotes the formation of thrombin during the process of thrombosis and coagulation in the vascular smooth muscle environment. Tissue Factor is categorized as a hemostasis-related activity in the CASM3C system modeling Th1 vascular smooth muscle inflammation. TF in the CASM3C system is regulated by the following pathways: Glucocorticoid R HDAC.

Urokinase plasminogen activator receptor (uPAR/CD87) is a cell surface receptor for urokinse plasminogen activator {uPA) involved in the regulation of pericellular proteolysis, cell migration, cancer cell invasion. and angiogenesis. uPAR is categorized as a tissue remodeling-related activity in the CASM3C system modeling Th1 vascular smooth muscle inflammation. uPAR in the CASM3C system is regulated by the following pathways: EGFR Glucocorticoid R HDAC, Histamine H1 R. JAK.

Interleukin-a {IL-8/CXCL8) is a chemokine that mediates neutrophil recruitment into acute inflammatory sites. IL-8 is categorized as an inflammation-related activity in the CASM3C system modeling Th1 vascular smooth muscle inflammation. IL-8 in the CASM3C system is regulated by the following pathways: Glucocorticoid R. IKK2.

Monokine induced by gamma interferon {M IG/CXCL9) is a chemokine that mediates T cell recruitment. MIG is categorized as an inflammation-related activity in the CASM3C system modeling Th1 vascular smooth muscle inflammation. MIG in the CASM3C system is regulated by the following pathways: HDAC, IKK2. JAK.

HLA-DR is a cell surface heterodimer involved in antigen presentation and is involved in T cell activation and immune responses. HLA-DR is categorized as an immunomodulatory-related activity in the CASM3C system modeling Th1 vascular smooth muscle inflammation. HLA-DR in the CASM3C system is regulated by the following pathways: HDAC. JAK. p38 MAPK.

Interleukin-6 {IL-6) is a secreted proinflammatory cytokine and acute phase reactant. Secreted IL-6 (sIl-6) is categorized as an inflammation-related activity in the CASM3C system modeling Th1 vascular smooth muscle inflammation. IL-6 in the CASM3C system is regulated by the following pathways: Glucocorticoid R HDAC. IKK2. Vitamin DR.

Low density lipoprotein receptor {LDLR) in the CASM3C system modeling Th1 vascular smooth muscle inflammation is a cell surface receptor involved in cholesterol regulation that mediates endocytosis of low-density lipoprotein {LDL).

Macrophage colony-stimulating factor {M-CSF) is a secreted and cell surface cytokine that mediates macrophage differentiation. M-CSF is categorized as an immunomodulatory-related activity in the CASM3C system modeling Th1 vascular smooth muscle inflammation. M-CSF in the CASM3C system is regulated by the following pathways: Glucocorticoid R. JAK.

Plasminogen activator inhibitor-1 (PAI-I} is a serine proteinase inhibitor and inhibitor of tissue plasminogen activator (tPA) and urokinase {uPA) and is involved in tissue remodeling and fibrinolysis. PAI-I is categorized as a t issue remodeling-related activity in the CASM3C system modeling Th1 vascular smooth muscle inflammation.

Proliferation in the CASM3C system is a measure of coronary artery smooth muscle cell proliferation which is important to the process of vascular biology and restenosis. Proliferation in the CASM3C system is regulated by the following pathways: EGFR. HDAC. HMG-CoA Reductase, Histamine H1 R. IKK2. Microtubule, PI3K. RAR/RXR. mTOR.

Serum Amyloid A {SAA) is a member of the apolipoprotein family that is an acute phase reactant. SAA is categorized as an inflammation-related activity in the CASM3C system modeling Th1 vascular smooth muscle inflammation. SAA in the CASM3C system is regulated by the following pathways: Glucocorticoid R HDAC.

SRB in the CASM3C system is a measure of the total protein content of coronary artery smooth muscle cells. Cell viability of adherent cells is measured by Sulforhodamine B (SRB) staining. a method that determines cell density by measuring total protein content of test wells. SRB in the CASM3C system is regulated by the following pathways: Histamine H1 R.

Monocyte chemoattractant protein-1 (MCP-1/CCL2) is a chemoattractant cytokine (chemokine) that regulates the recruitment of monocytes and T cells into sites of inflammation. MCP-1 is categorized as an inflammation-related activity in the HDF3CGF system modeling Th1 inflammation involved in wound healing and matrix remodeling of the skin. MCP-1 in the HDF3CGF system is regulated by the following pathways: IKK2, PI 3K. RAR/RXR. Src.

Vascular Cell Adhesion Molecule 1 (VCAM-1/CD106) is a cell adhesion molecule that mediates adhesion of monocytes and T cells to endothelial cells. VCAM-1 is categorized as an inflammation-related activity in the HDF3CGF system modeling Th1 inflammation involved in wound healing and matrix remodeling of the skin. VCAM-1 in the HDF3CGF system is regulated by the following pathways: IKK2. JAK. MEK. RAR/RXR. Src. Vitamin D R.

Intercellular Adhesion Molecule 1 {ICAM-1/CD54} is a cell adhesion molecule that mediates leukocyte-endothelial cell adhesion and leukocyte recruitment. ICAM-1 is categorized as an inflammation-related activity in the HDF3CGF system modeling Th1 inflammation involved in wound healing and matrix remodeling of the skin. ICAM-1 in the HDF3CGF system is regulated by the following pathways: RAR/RXR.

Collagen I is involved in tissue remodeling and fibrosis, and is the most common fibrillar collagen that is found in skin. bone, tendons, and other connective tissues. Collagen I is categorized as a tissue remodeling-related activity in the HDF3CGF system modeling Th1 inflammation involved in wound healing and matrix remodeling of the skin.

Collagen III is an extracellular matrix protein and fibrillar collagen found in extensible connective tissues (skin, lung and vascular system) and is involved in cell adhesion, cell migration, t issue remodeling. Collagen III is categorized as a tissue remodeling-related activity in the HDF3CGF system modeling Th1 inflammation involved in wound healing and matrix remodeling of the skin. Collagen III in the HDF3CGF system is regulated by the following pathways: RAR/RXR.

Interferon-gamma-inducible protein 1 o {IP-10/CXCL 1 0) is a chemokine that mediates T cell, monocyte and dendritic cell chemotaxis. IP-1 o is categorized as an inflammation-related activity in the HDF3CGF system modeling Th1 inflammation involved in wound healing and matrix remodeling of the skin. IP-1 o in the HDF3CGF system is regulated by the following pathways: HDAC. Histamine H1 R. IKK2. JAK. RAR/RXR. Src.

Interferon-inducible T Cell Alpha Chemoattractant (I-TAC/CXCL 11) is a chemokine that mediates T cell and monocyte chemotaxis. I-TAC is categorized as an inflammation-related activity in the HDF3CGF system modeling Th1 inflammation involved in wound healing and matrix remodeling of the skin. I-TAC in the HDF3CGF system is regulated by the following pathways: HDAC, JAK. RAR/RXR.

Interleukin-8 (IL-8/CXCL8) is a chemokine that mediates neutrophil recruitment into acute inflammatory sites. IL-8 is categorized as an inflammation-related activity in the HDF3CGF system modeling Th1 inflammation involved in wound healing and matrix remodeling of the skin. IL-8 in the HDF3CGF system is regulated by the following pathways: IKK2.

Monokine induced by gamma interferon {MIG/CXCL9) is a chemokine that mediates T cell recruitment. MIG is categorized as an inflammation-related activity in the HDF3CGF system modeling Th1 inflammation involved in wound healing and matrix remodeling of the skin. MIG in the HDF3CGF system is regulated by the following pathways: HDAC. JAK. RAR/RXR. Src.

Epidermal growth factor receptor (EGFR) is a cell surface receptor for epidermal growth factor involved in cell proliferation, cell differentiation, tissue remodeling and tumor growth. EGFR is categorized as a tissue remodeling-related activity in the HDF3CGF system modeling Th1 inflammation involved in wound healing and mat r ix remodeling of the skin. EGFR in the HDF3CGF system is regulated by the following pathways: EGFR.

Macrophage colony-stimulating factor {M-CSF) is a secreted and cell surface cytokine that mediates macrophage differentiation. M-CSF is categorized as a tissue remodeling-related activity in the HDF3CGF system modeling Th1 inflammation involved in wound healing and matrix remodeling of the skin. M-CSF in the HDF3CGF system is regulated by the following pathways: IKK2, JAK. MEK. RAR/RXR. Sr c.

Matrix metalloproteinase-1 {MMP-1) is an interstitial collagenase that degrades collagens I, II and III and is involved in the process of tissue remodeling. MMP-1 is categorized as a tissue remodeling-related activity in the HDF3CGF system modeling Th1 inflammation involved in wound healing and matrix remodeling of the skin. MMP-1 in the HDF3CGF system is regulated by the following pathways: MEK. RAR/RXR. Src.

Plasminogen activator inhibitor-1 (PAI-I) is a serine proteinase inhibitor and inhibitor of tissue plasminogen activator (tPA) and urokinase (LIPA) and is involved in tissue remodeling and fibrinolysis. PAI-I is categorized as a tissue remodeling-related activity in the HDF3CGF system modeling Th1 inflammation involved in wound healing and matrix remodeling of the skin. PAI-1 in the HDF3CGF system is regulated by the following pathways: EGFR. Histamine H1 R. MEK. PDE4. P13K. RAR/RXR. Src. mTOR.

Proliferation in the HDF3CGF system is a measure of dermal fibroblast proliferation which is important to the process of wound healing and fibrosis. Proliferation in the HDF3CGF system is regulated by the following pathways: EGFR. HDAC. HMG-CoA Reductase. Histamine H1 R. IKK2. Microtubule, P13K. RAR/RXR. Src. mTOR.

SRB in the HDF3CGF system is a measure of the total protein content of dermal fibroblasts. Cell viability of adherent cells is measured by Sulforhodamine B {SRB) staining. a method that determines cell density by measuring total protein content of test wells. SRB in the HDF3CGF system is regulated by the following pathways: IKK2. RAR/RXR.

TIMP-1 is a tissue inhibitor of matrix metalloprotease-7 (MMP-7) and other MMPs and is involved in tissue remodeling. angiogenesis and fibrosis. TIMP-1 is categorized as a tissue remodeling-related activity in the HDF3CGF system modeling Th1 inflammation involved in wound healing and matrix remodeling of the skin. TIMP-1 in the HDF3CGF system is regulated by the following pathways: Vitamin D R.

TIMP-2 is a tissue inhibitor of matrix metalloproteases and is involved in tissue remodeling. angiogenesis and fibrosis. TIMP-2 is categorized as a tissue remodeling-related activity in the HDF3CGF system modeling Th1 inflammation involved in wound healing and matrix remodeling of the skin.

Monocyte chemoattractant protein-1 (MCP-1/CCL2) is a chemoattractant cytokine (chemokine) that regulates the recruitment of monocytes and T cells into sites of inflammation. MCP-1 is categorized as an inflammation-related activity in the KF3CT system modeling Th1 cutaneous inflammation. MCP-1 in the KF3CT system is regulated by the following pathways: IKK2.

Intercellular Adhesion Molecule 1 (ICAM-1/CD54) is a cell adhesion molecule that mediates leukocyte-endothelial cell adhesion and leukocyte recruitment. ICAM-1 is categorized as an inflammation-related activity in the KF3CT system modeling Th1 cutaneous inflammation. ICAM-1 in the KF3CT system is regulated by the following pathways: JAK.

Interferon-gamma-inducible protein 1 o {IP-10/CXCL 1 0) is a chemokine that mediates T cell, monocyte and dendritic cell chemotaxis. IP-1 o is categorized as an inflammation-related activity in the KF3CT system modeling Th1 cutaneous inflammation. IP-10 in the KF3CT system is regulated by the following pathways: JAK RAR/RXR. Sr c.

Interleukin-8 (IL-8/CXCL8) is a chemokine that mediates neutrophil recruitment into acute inflammatory sites. IL-8 is categorized as an inflammation-related activity in the KF3CT system modeling Th1 cutaneous inflammation. IL-8 in the KF3CT system is regulated by the following pathways: IKK2.

Monokine induced by gamma interferon (MIG/CXCL9) is a chemokine that mediates T cell recruitment. MIG is categorized as an inflammation-related activity in the KF3CT system modeling Th1 cutaneous inflammation. MIG in the KF3CT system is regulated by the following pathways: JAK. Src.

Interleukin-1 alpha (IL-1a) is a secreted proinflammatory cytokine involved in endothelial cell activation and neutrophil recruitment. Secreted IL-1a {sIl-1a) is categorized as an inflammation-related activity in the KF3CT system modeling Th1 cutaneous inflammation. IL-1a in the KF3CT system is regulated by the following pathways: IKK2. Src.

Matrix metalloproteinase-9 {MMP-9) is a gelatinase B that degrades collagen IV and gelatin and is involved in cutaneous remodeling. MMP-9 is categorized as a tissue remodeling-related activity in the KF3CT system modeling Th1 cutaneous inflammation. MMP-9 in the KF3CT system is regulated by the following pathways: HDAC. Histamine H1 R. IKK2. Src.

Plasminogen activator inhibitor-1 (PAI-I) is a serine proteinase inhibitor and inhibitor of tissue plasminogen activator (tPA) and urokinase (LIPA) and is involved in tissue remodeling and fibrinolysis. PAI-I is categorized as a t issue remodeling-related activity in the KF3CT system modeling Th1 cutaneous inflammation. PAI-1 in the KF3CT system is regulated by the following pathways: P13K. RAR/RXR.

SRB in the KF3CT system is a measure of the total protein content of keratinocytes and dermal fibroblasts. Cell viability of adherent cells is measured by Sulforhodamine B (SRB) staining. a method that determines cell density by measuring total protein content of test wells.

TIMP-2 is a tissue inhibitor of matrix metalloproteases and is involved in tissue remodeling. angiogenesis and fibrosis. TIMP-2 is categorized as a tissue remodeling-related activity in the KF3CT system modeling Th1 cutaneous inflammation. TIMP-2 in the KF3CT system is regulated by the following pathways: IKK2. Src.

Urokinase plasminogen activator (uPA) is a serine protease with thrombolytic activity. Triggers fibrinolysis and extracellular matrix degradation. uPA is categorized as a tissue remodeling-related activity in the KF3CT system modeling Th1 cutaneous inflammation. uPA in the KF3CT system is regulated by the following pathways: HDAC. IKK2. Src.

Alpha-smooth muscle actin {a-SMA) is a protein involved in muscle contraction, cell motility, structure and integrity and is a marker for activated myofibroblast alpha-SM Actin phenotype. a-SMA is categorized as a t issue remodeling-related activity in the MyoF system modeling pulmonary myofibroblast development. aSMA in the MyoF system is regulated by the following pathways: RAR/RXR. Src.

Basic fibroblast growth factor (bFGF) is a pro-fibrotic growth factor that drives fibroblast proliferation, migration and fibronectin synthesis. bFGF is categorized bFGF as a tissue remodeling-related activity in the MyoF system modeling pulmonary myofibroblast development. bFGF in the MyoF system is regulated by the following pathways: HDAC.

Vascular Cell Adhesion Molecule 1 {VCAM-1/CD106} is a cell adhesion molecule that mediates adhesion of monocytes and T cells to endothelial cells. VCAM-1 is categorized as an inflammation-related activity in the MyoF system modeling pulmonary myofibroblast development. VCAM-1 in the MyoF system is regulated by the following pathways: Histamine H 1 R. IKK2. P13K. RAR/RXR. TNF-alpha.

Collagen I is involved in tissue remodeling and fibrosis and is the most common fibrillar collagen that is found in skin, bone, tendons and other connective Collagen I tissues. Collagen I is categorized a tissue remodeling-related activity in the MyoF system modeling pulmonary myofibroblast development. Collagen I in the MyoF system is regulated by the following pathways: RAR/RXR.

Collagen III is an extracellular matrix protein and fibrillar collagen found in extensible connective tissues {skin. lung and vascular system) and is involved in cell Collagen III adhesion, cell migration, tissue remodeling. Collagen III is categorized a t issue remodeling-related activity in the MyoF system modeling pulmonary myofibroblast development. Collagen III in the MyoF system is regulated by the following pathways: HDAC. Histamine H1 R. IKK2, RAR/RXR.

Collagen IV is the major structural component of the basal lamina. Collagen IV is categorized a tissue remodeling-related activity in the MyoF system modeling pulmonary myofibroblast development.

Interleukin-8 {IL-8/CXCLB) is a chemokine that mediates neutrophil recruitment into acute inflammatory sites. IL-8 is categorized as an inflammation-related activity in the MyoF system modeling pulmonary myofibroblast development. IL-8 in the MyoF system is regulated by the following pathways: Glucocorticoid R IKK2. TNF-alpha, Vitamin D R, p38 MAPK.

Decorin is a proteoglycan that is a component of connective tissue and is involved in collagen and matrix assembly. Decorin is categorized as a tissue remodeling-related activity in the MyoF system modeling pulmonary myofibroblast development.

Matrix metalloproteinase-1 (MMP-1) is an interstitial collagenase that degrades collagens I. II and III and is involved in the process of t issue remodeling. MMP-1 is categorized as a tissue remodeling-related activity in the MyoF system modeling pulmonary myofibroblast development. MMP-1 in the MyoF system is regulated by the following pathways: IKK2. Microtubule.

Plasminogen activator inhibitor-1 {PAI-1) is a serine proteinase inhibitor and inhibitor of tissue plasminogen activator (tPA) and urokinase (LIPA) and is involved in tissue remodeling and fibrinolysis. PAI-I is categorized as a tissue remodeling-related activity in the MyoF system modeling pulmonary myofibroblast development. PAI-1 in the MyoF system is regulated by the following pathways: P13K. Src.

SRB in the MyoF system is a measure of the total protein content of lung fibroblasts. Cell viability of adherent cells is measured by Sulforhodamine B {SRB) staining. a method that determines cell density by measuring total protein content of test wells. SRB in the MyoF system is regulated by the following pathways: HOAC, Histamine H1 R, IKK2. RAR/RXR.

TIMP-1 is a tissue inhibitor of matrix metalloprotease-7 (MMP-7) and other MMPs and is involved in tissue remodeling. angiogenesis and fibrosis. TIMP-1 is categorized as a tissue remodeling-related activity in the MyoF system modeling pulmonary myofibroblast development. TIMP-1 in the MyoF system is regulated by the following pathways: Glucocorticoid R.

Monocyte chemoattractant protein-1 (MCP-1/CCL2) is a chemoattractant cytokine {chemokine) that regulates the recruitment of monocytes and T cells into sites of inflammation. MCP-1 is categorized as an inflammation-related activity in the /Mphg system modeling macrophage-driven Th1 vascular inflammation. MCP-1 in the IMphg system is regulated by the following pathways: Glucocorticoid R. IKK2.

Macrophage inflammatory protein 1 alpha (MIP-1 a/CCL3) is a pro-inflammatory chemokine that mediates leukocyte recruitment to sites of inflammation. MIP-1 a is categorized as an inflammation-related activity in the /Mphg system modeling macrophage-driven Th1 vascular inflammation. MIP-1 a in the IMphg system is regulated by the following pathways: Glucocorticoid R. IKK2.

Vascular Cell Adhesion Molecule 1 (VCAM-1/CD106) is a cell adhesion molecule that mediates adhesion of monocytes and T cells to endothelial cells. VCAM-1 is categorized as an inflammation-related activity in the /Mphg system modeling macrophage-driven Th1 vascular inflammation. VCAM-1 in the IMphg system is regulated by the following pathways: IKK2. TNF-alpha.

CD40 is a cell surface adhesion receptor and costimulatory receptor for T cell activation that is expressed on antigen presenting cells, endothelial cells, smooth muscle cells, fibroblasts and epithelial cells. CD40 is categorized as an immunomodulatory-related activity in the /Mphg system modeling macrophage-driven Th1 vascular inflammation. CD40 in the IMphg system is regulated by the following pathways: IKK2.

E-Selectin/CD62E is a cell adhesion molecule expressed only on endothelial cells that mediates leukocyte-endothelial cell interactions. E-Selectin is categorized as an inflammation-related activity in the /Mphg system modeling macrophage-driven Th1 vascular inflammation. E-selectin in the IMphg system is regulated by the following pathways: Glucocorticoid R. HMG-CoA Reductase, IKK2, TNF-alpha, p38 MAPK.

C069 is a cell surface activation antigen that is induced early during immune activation and is involved in macrophage activation. C069 is categorized as an immunomodulatory-related activity in the /Mphg system modeling macrophage-driven Th1 vascular inflammation.

Interleukin-8 (IL-8/CXCL8) is a chemokine that mediates neutrophil recruitment into acute inflammatory sites. IL-8 is categorized as an inflammation-related activity in the /Mphg system modeling macrophage-driven Th1 vascular inflammation. IL-8 in the IMphg system is regulated by the following pathways: Glucocorticoid R. IKK2, Vitamin D R. p38 MAPK_

Interleukin-1 alpha (IL-1 a} is a secreted proinflammatory cytokine involved in endothelial cell activation and neutrophil recruitment. Secreted IL-1 a (s Il-1 a) is categorized as an inflammation-related activity in the /Mphg system modeling macrophage-driven Th1 vascular inflammation. IL-1a in the IMphg system is regulated by the following pathways: Glucocorticoid R. IKK2.

Macrophage colony-stimulating factor (M-CSF) is a secreted and cell surface cytokine that mediates macrophage differentiation. M-CSF is categorized as an immunomodulatory-related activity in the /Mphg system modeling macrophage-driven Th 1 vascular inflammation.

Interleukin-10 {IL-10) is a secreted anti-inflammatory cytokine. Secreted IL-10 {sIl-10) is categorized as an immunomodulatory-related activity in the /Mphg system modeling macrophage-driven Th1 vascular inflammation. sIl-10 in the IMphg system is regulated by the following pathways: EGFR Glucocorticoid R HDAC, Histamine H1 R, JAK. MEK. PDE4, RAR/RXR, Src. TNF-alpha, mTOR, p38 MAPK.

SRB in the /Mphg system is a measure of the total protein content of venular endothelial cells and macrophages. Cell viability of adherent cells is measured by Sulforhodamine B (SRB) staining. a method that determines cell density by measuring total protein content of test wells.

SRB-Mphg in the /Mphg system is a measure of the total protein content of macrophages alone. Cell viability of adherent cells is measured by Sulforhodamine B (SRB) staining a method that determines cell density by measuring total protein content of test wells.

In some embodiments, the compounds disclosed in the invention are used to treat a disease or condition by immunomodulation wherein the said compound acts on one or more of the biological targets selected from CB2, sIL-10, CD40, M-CSF, CD40, sIL-17A, sIgG or sIL-17F.

In some embodiments, the compounds disclosed in the invention are used to treat a disease or condition by immunomodulation wherein the said compound decreases pro-inflammatory cytokines and acts on one or more of the biological targets selected from CB2, TNFα, VCAM-1, MCP-1, IL-6, IL-31, IL-17, IL-12, IL-1α, IL-1β, I-TAC or MIG.

In some embodiments, the compounds disclosed in the invention are used to treat a disease or condition by affecting hemostasis wherein the said compounds acts on one or more of the biological targets selected from TF or VEGFR2.

In some embodiments, the compounds disclosed in the invention are antiproliferative wherein the said compounds are antiproliferative to human B cells, T cells, fibroblasts or endothelial cells. some embodiments, the compounds disclosed in the invention affect tissue remodeling activity wherein the said compounds acts on one or more of the biological targets selected from PAI-1, MMP1, EGFR, uPAR, tPA or MMP9.

In some embodiments, the compounds disclosed in the invention are anti-fibrotic wherein the said compounds acts on one or more of the biological targets selected from PAI-1, TIMP-1, type I and type III collagens or αSMA.

1. A compound represented by the following structural Formula II, a pharmaceutically acceptable salt, solvate, hydrate, polymorph, enantiomer, diastereomer, geometric isomer, racemate, tautomer, rotamer, atropisomer, isotopic variation, or N-oxide thereof: 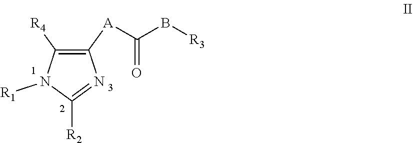
wherein:
A is N(R5) or a direct bond,
B is N(R5),
R5 is hydrogen, OH, alkyl or substituted alkyl,
Z5 is optionally present and if present is —C(═O)—
R1 is -T-(CH₂)ₘ-Q-(CH₂)ₙ—Z,
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C;
Z is selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
X₄, X₅, and X₆ each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀, wherein
X₈ and X₁₀ are each independently H or alkyl, wherein
m is an integer from 0 to 7
j is an integer from 0 to about 6, or
k is an integer from 0 to about 2; or
R1 is -T-(CH₂)ₘ-Q-(CH₂)ₙ—Z;
m and n independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₘ-Q-(CH₂)ₙ—Z;
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₘ-Q-(CH₂)ₙ—Z;
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C; and
Z is selected from the group consisting of 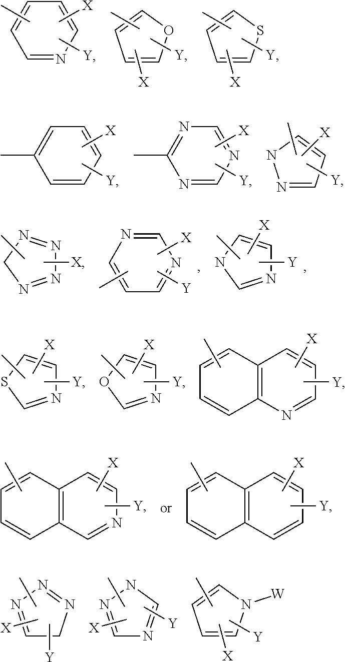
wherein X and Y are each independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
X₄, X₅, and X₆ each independently selected from H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀ wherein
X₈ and X₁₀ are each independently H or alkyl, wherein m is an integer from 0 to 7;
j is an integer from 0 to about 6,
k is an integer from 0 to about 2,
W is H or alkyl; or
R1 is -T-(CH₂)ₘ-Q-(CH₂)ₙ—Z;
m and n independently comprises an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-(CH₂)ₘ-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n are independently an integer from 0 to about 7;
Q is CH═CH or C≡C;
Z comprises 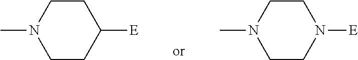
E is selected from the group consisting of a C1 to about C4, linear or branched alkyl group, a phenyl group, a substituted phenyl group, a benzyl group or a substituted benzyl group; or
R1 is -T-(CH₂)ₘ-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n are independently an integer from 0 to about 7;
Q is CH═CH or C≡C; and
Z is selected from the group consisting of 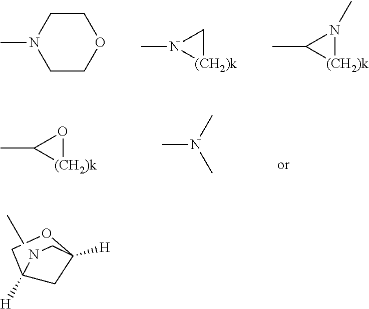
k is an integer from 1 to about 5, and
A₁ and A₂ are each independently selected from the group consisting of a C1 to about C4 alkyl group, a phenyl group or a substituted phenyl group; or
R1 is —(CH₂)ₙ—Z;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6, or
k is an integer from 0 to about 2; or
R1 is —(CH₂)ₙ—Z;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of 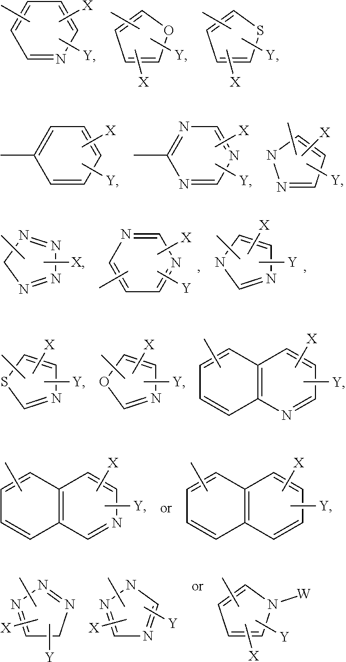
wherein X and Y are independently selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀ wherein X₉ and X₁₀ are each independently H or alkyl,
wherein m is an integer from 0 to 7,
j is an integer from 0 to about 6, or
k is an integer from 0 to about 2 W is H or alkyl; or
R1 is —(CH₂)ₙ—Z;
n is an integer from 0 to about 7; and
Z is selected from the group consisting of a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R1 is —(CH₂)ₙ—Z;
n is selected from the group consisting of an integer from 0 to about 7; and
Z comprises an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 is —(CH₂)ₘ-Q₁-(CH₂)ₙ—Z;
Q₁ is selected from the group consisting of NH, O, S, —CH═CH—, —C≡C—, —CO, SO₂ and OSO₂;
m is an integer from 1 to about 7;
n is an integer from 0 to about 7; and
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, or —CX₉═CHX₁₀ wherein X₉ and X₁₀ each independently H or alkyl,
wherein m is an integer from 0 to 7,
j is an integer from 0 to about 6, or
k is an integer from 0 to about 2; or
R1 is -Q₂-(CH₂)ₙ—Z;
Q₂ is optionally present and if present is selected from the group consisting of —CH₂—NH, —CH₂—O, —CH₂—S, —CH₂—SO₂ or —CH₂—OSO₂;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
X₁ and X₂ are each independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀ wherein
X₉ and X₁₀ are each independently H or alkyl,
wherein m is an integer from 0 to 7,
j is an integer from 0 to about 6,
k is an integer from 0 to about 2; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a bicyclic ring, a heterobicyclic ring, a tricyclic ring, a heterotricyclic ring, a polycyclic ring and a heteropolycyclic ring; or
R1 is —(CH₂)ₘ-Q₁-(CH₂)ₙ—Z;
Q₁ is selected from the group consisting of NH, O, S, CH═CH, C≡C, CO, SO₂ or OSO₂;
m is an integer from 1 to about 7;
n is an integer from 0 to about 7;
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 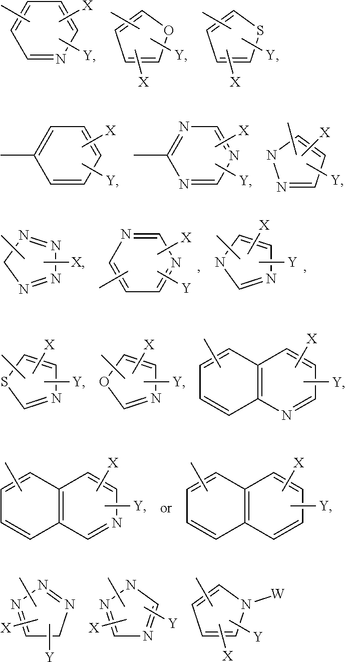
wherein X and Y are each independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀ wherein
X₉ and X₁₀ are each independently H or alkyl,
wherein m is an integer from 0 to 7,
j is an integer from 0 to about 6,
k is an integer from 0 to about 2,
W comprises H or alkyl; or
R1 is —(CH₂)ₘ-Q₁-(CH₂)ₙ—Z;
Q₁ is selected from the group consisting of NH, O, S, CH═CH, C≡C, CO, SO₂ or OSO₂;
m is an integer from 1 to about 7;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 is —(CH₂)ₘ-Q₁-(CH₂)ₙ—Z;
Q₁ is selected from the group consisting of NH, O, S, CH═CH, C≡C, CO, SO₂ or OSO₂;
m is an integer from 1 to about 7;
n is an integer from 0 to about 7; and
Z is selected from the group consisting of 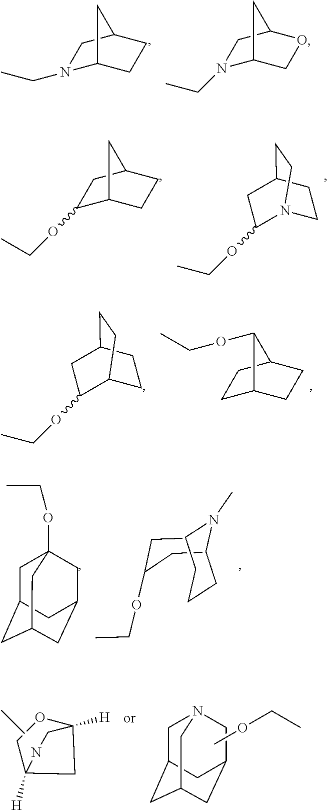
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀ wherein
X₉ and X₁₀ are each independently H or alkyl,
wherein m is an integer from 0 to 7,
j is an integer from 0 to about 6,
k is an integer from 0 to about 2; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of 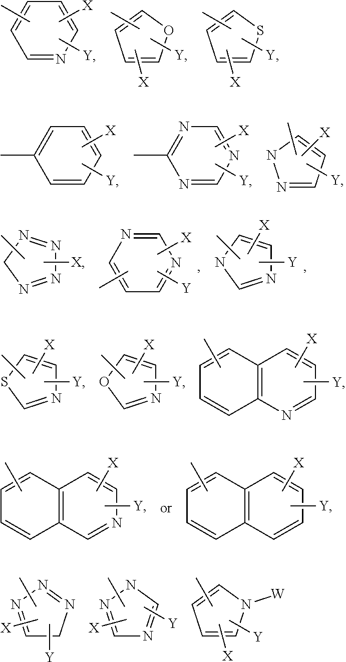
wherein X and Y are independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀ wherein
X₉ and X₁₀ are independently H or alkyl,
wherein m is an integer from 0 to 7,
j is an integer from 0 to about 6,
k is an integer from 0 to about 2
W is H or alkyl; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-Q-(CH₂)ₙ—Z,
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6, or
k is an integer from 0 to about 2; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 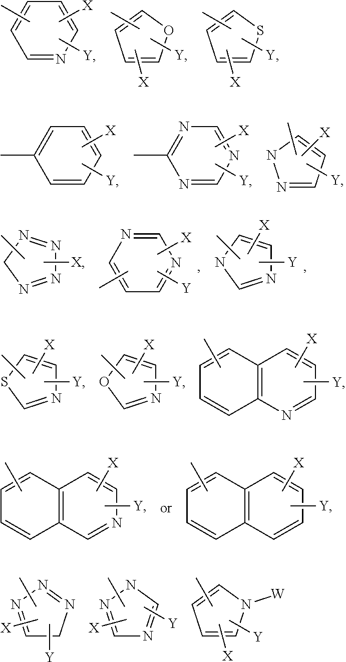
wherein X and Y are independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6,
k is an integer from 0 to about 2,
W is H or alkyl; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
each n is independently an integer from 0 to about 7;
Q is CH═CH or C≡C;
Z is 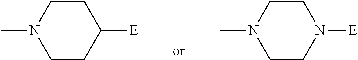
E is selected from the group consisting of a C1 to about C4, linear or branched alkyl group, a phenyl group, a substituted phenyl group, a benzyl group and a substituted benzyl group; or
R1 is -T-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each n is independently an integer from 0 to about 7;
Q is CH═CH or C≡C; and
Z is selected from the group consisting of 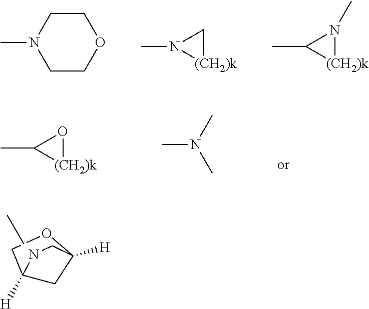
k is an integer from 1 to about 5, and
A₁ and A₂ are each independently selected from the group consisting of a C1 to about C4 alkyl group, a phenyl group and a substituted phenyl group;
R2 is —(CH₂)ₙ—Z;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
X₄, X₅, and X₆ are independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6, or
k is an integer from 0 to about 2; or
R2 is —(CH₂)ₙ—Z;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of 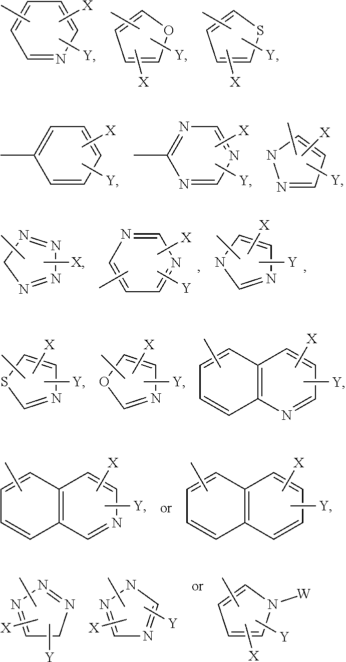
wherein X and Y are each independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6, or
k is an integer from 0 to about 2,
W comprises H or alkyl; or
R2 is —(CH₂)ₙ—Z;
n is an integer from 0 to about 7; and
Z is selected from the group consisting of a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R2 is —(CH₂)ₙ—Z;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 is —(CH₂)ₘ-Q₁-(CH₂)ₙ—Z;
Q₁ is selected from the group consisting of NH, O, S, —CH═CH—, —C≡C—, —CO, SO₂ or OSO₂;
m is an integer from 1 to about 7;
n is an integer from 0 to about 7; and
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
Wherein m is an integer from 0 to 7,
j is an integer from 0 to about 6, or
k is an integer from 0 to about 2; or
R2 is -Q₂-(CH₂)ₙ—Z;
Q₂ is optionally present and if present is selected from the group consisting of —CH₂—NH, —CH₂—O, —CH₂—S, —CH₂—SO₂ or —CH₂—OSO₂;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6,
k is an integer from 0 to about 2; or
R2 is —(CH₂)ₘ-Q₁-(CH₂)ₙ—Z;
Q₁ is selected from the group consisting of NH, O, S, CH═CH, C≡C, CO, SO₂ or OSO₂;
m is an integer from 1 to about 7;
n is an integer from 0 to about 7;
Z is selected from the group consisting of a bicyclic ring, a heterobicyclic ring, a tricyclic ring, a heterotricyclic ring, a polycyclic ring or a heteropolycyclic ring; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ or OSO₂;
m is an integer from 1 to about 7;
n is an integer from 0 to about 7;
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ or OSO₂;
m is an integer from 1 to about 7;
n is an integer from 0 to about 7; and
Z is selected from the group consisting of 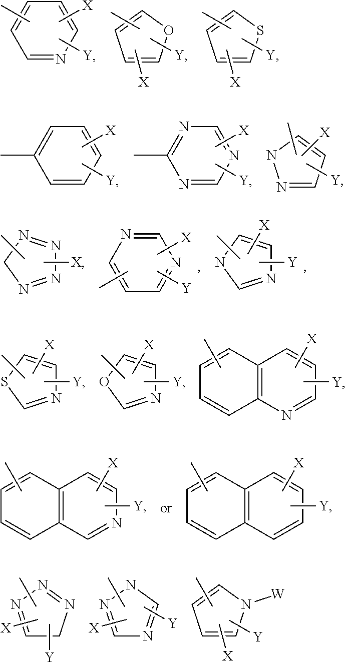
wherein X and Y are each independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6,
k is an integer from 0 to about 2
W is H or alkyl; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 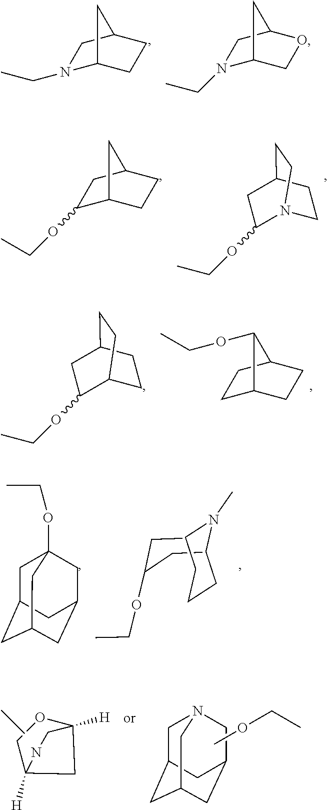
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6,
k is an integer from 0 to about 2; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of 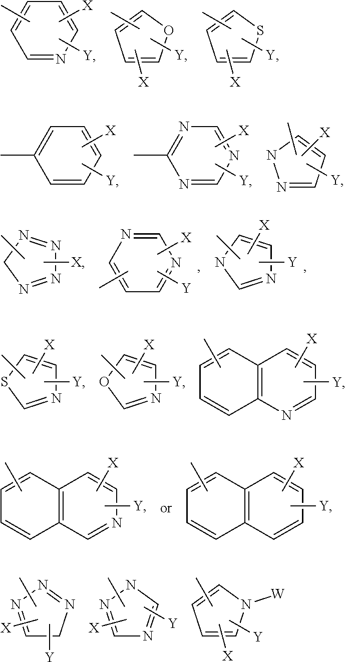
wherein X and Y are each independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, or —CX₉═CHX₁₀ wherein
X₉ and X₁₀ are each independently H or alkyl,
wherein m is an integer from 0 to 7,
j is an integer from 0 to about 6,
k is an integer from 0 to about 2,
W comprises H or alkyl; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R2 is -T-(CH₂)ₘ-Q₁-(CH₂)ₙ—Z;
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ is selected from the group consisting of NH, O, S, CH═CH, C≡C, CO, SO₂ and OSO₂; and
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, or O-alkyl-X₇ wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of a H, alkyl, carbocyclic ring, heterocyclic ring, aromatic ring, heteroaromatic ring, and —CX₉═CHX₁₀, wherein
X₉ and X₁₀ are each independently H or alkyl,
wherein m is an integer from 0 to 7,
j is an integer from 0 to about 6,
k is an integer from 0 to about 2; or
R2 is -T-(CH₂)ₘ-Q₁-(CH₂)ₙ—Z;
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₘ-Q₁-(CH₂)ₙ—Z;
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ is selected from the group consisting of NH, O, S, CH═CH, C≡C, CO, SO₂ and OSO₂; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₘ-Q₁-(CH₂)ₙ—Z;
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ is selected from the group consisting of N, O, S, CH═CH, C≡C, CO, SO₂ and OSO₂; and
Z is selected from the group consisting of 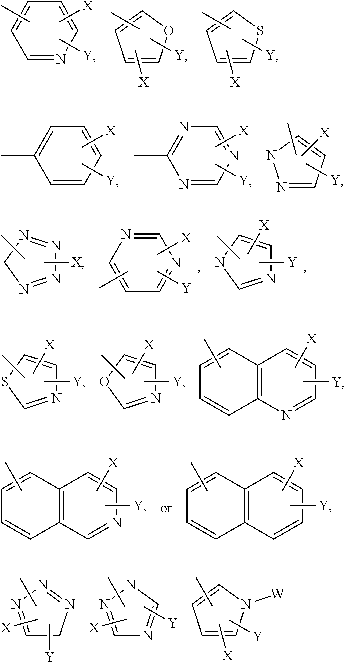
wherein X and Y are each independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl,
wherein m is an integer from 0 to 7,
j is an integer from 0 to about 6,
k is an integer from 0 to about 2,
W is H or alkyl; or
R2 is -T-(CH₂)ₘ-Q₁-(CH₂)ₙ—Z;
m and n are independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ is selected from the group consisting of NH, O, S, CH═CH, C≡C, CO, SO₂ and OSO₂; and
Z is selected from the group consisting of an unsaturated ring having 5 ring atoms and 0 to 2 independently selected heteroatoms as ring members fused to an unsaturated ring having 5 ring atoms and 0 to 2 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members, or
R2 is -T-(CH₂)ₘ-Q₁-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n are independently an integer from 0 to about 7;
Q₁ is selected from the group consisting of NH, O, S, CH═CH, C≡C, CO, SO₂ and OSO₂; and
Z is selected from the group consisting of 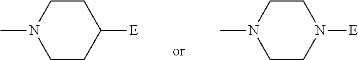
wherein
E is selected from the group consisting of a C1 to about C4, linear or branched alkyl group, a phenyl group, a substituted phenyl group, a benzyl group and a substituted benzyl group; or
R2 is -T-(CH₂)ₘ-Q₁-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
m and n are independently an integer from 0 to about 7;
Q₁ is selected from the group consisting of NH, O, S, CH═CH, C≡C, CO, SO₂ and OSO₂;
Z is selected from the group consisting of 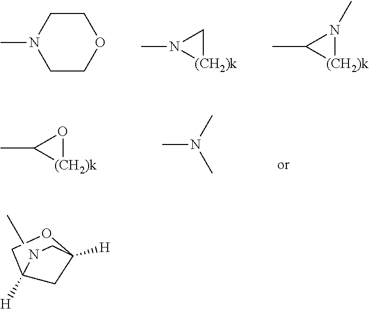
k is an integer from 1 to about 5,
A₁ and A₂ each independently selected from the group consisting of a C1 to about C4 alkyl group, a phenyl group and a substituted phenyl group;
R3 is 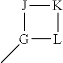
wherein G is selected from the group consisting of CH, C(CH₃), C(CN) and N,
L, K and J are each independently selected from the group consisting of (CH₂)ₙ, (CH₃)₂, C═O, 0, —CHOH, C(CH₃)OM₁, C(CH₂)ₙ(X)Y, NM₁, SO₂ SO and S,
and at least one of L, K or J is SO₂,
n is an integer from 0 to about 7;
M₁ is selected from the group consisting of H, alkyl, and C(O)M₂, wherein
M₂ is selected from the group consisting of H, alkyl, NM₃M₄, and OM₅, and M₃, M₄ and M₅ are independently H, OH or alkyl,
and X and Y are each independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
X₉ and X₁₀ are each independently H or alkyl,
wherein m is an integer from 0 to 7,
j is an integer from 0 to about 6, or
k is an integer from 0 to about 2;
R4 is selected from the group consisting of
H, halogen, CF₃, CF₂H, N₃, NCS, CN, NO₂, NX₁X₂, OH, ONO₂, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
X₉ and X₁₀ are each independently H or alkyl,
wherein m is an integer from 0 to 7,
j is an integer from 0 to about 6, or
k is an integer from 0 to about 2; or
R4 is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring or a heteropolycyclic ring; or
R4 is selected from the group consisting of 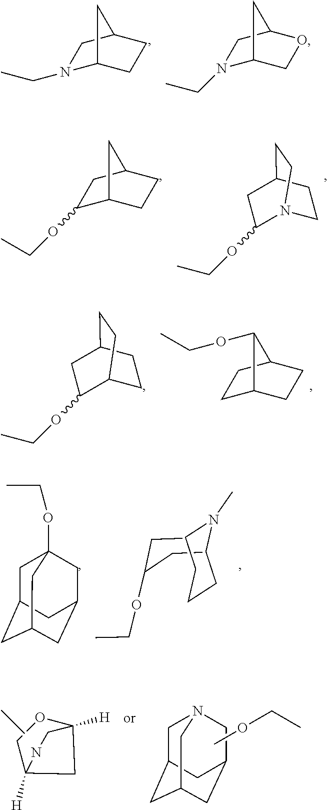
R4 is —(CH₂)_{d}—Z;
d is an integer from 1 to about 6;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
X₁ and X₂ are each independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
X₉ and X₁₀ are each independently H or alkyl,
wherein m is an integer from 0 to 7,
j is an integer from 0 to about 6,
k is an integer from 0 to about 2; or
R4 is —(CH₂)_{d}—Z;
d is an integer from 1 to about 6; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 is —(CH₂)_{d}—Z;
d is an integer from 1 to about 6; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, COX₈, wherein
X₈ is selected from the group consisting of H, alkyl, carbocyclic ring, heterocyclic ring, aromatic ring, heteroaromatic ring, or —CX₉═CHX₁₀ wherein
X₉ and X₁₀ are each independently H or alkyl,
wherein m is an integer from 0 to 7,
j is an integer from 0 to about 6, or
k is an integer from 0 to about 2; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 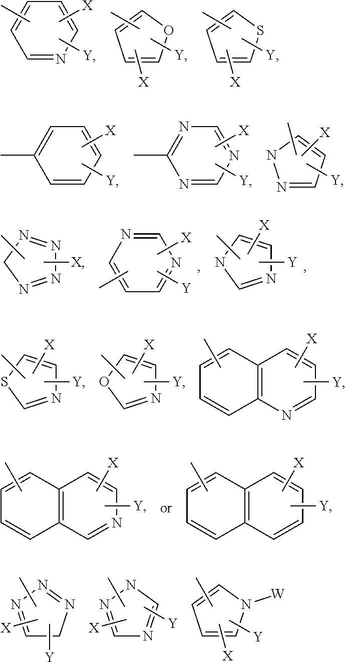
wherein X and Y are each independently selected from the group consisting of
H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO₂-alkyl, alkoxy, alkyl, alcohol, alkylmercapto, alkylamino, di-alkylamino, alkylsulfinyl or alkylsulfonyl, CX₄X₅X₆, —CH═CHX₈, —C≡CX₈;
X₁ and X₂ are each independently H or alkyl, or
X₁ and X₂ together form part of a heterocyclic ring having about 4 to about 7 ring members and optionally one additional heteroatom selected from O, N or S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
X₄, X₅, and X₆ are each independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, or —CX₉═CHX₁₀, wherein
X₉ and X₁₀ are each independently H or alkyl
wherein m is an integer from 0 to 7,
j is an integer from 0 to about 6,
k is an integer from 0 to about 2, and
W comprises H or alkyl.

2-7. (canceled)

8. The compound of claim 1, a pharmaceutically acceptable salt, solvate, hydrate, polymorph, enantiomer, diastereomer, geometric isomer, racemate, tautomer, rotamer, atropisomer, isotopic variation, or N-oxide thereof, wherein R3 is represented by Formula VIII:

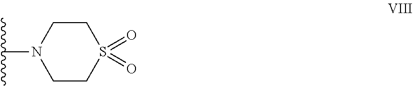 [SMILES: *N1CCS(=O)(=O)CC1]

9. The use of a compound of claim 35, wherein the compound is used to treat an inflammatory condition related to liver disease selected from hepatitis C, alcoholic or non-alcoholic steatohepatitis, fibrosis, alcoholic or non-alcoholic cirrhosis and cancer.

10. The use of a compound of claim 35, wherein the compound is used to treat an inflammatory condition related to lung disease selected from idiopathic, genetic, radiation-induced, environmental agent induced or chemically induced pulmonary fibrosis.

11. The use of a compound of claim 10, wherein the condition is respiratory distress syndrome.

12. The use of a compound of claim 11, wherein the condition is acute respiratory distress syndrome caused by COVID-19.

13. The use of a compound of claim 35, wherein the compound is used to treat an inflammatory condition related to kidney disease selected from diabetic nephropathy, renal fibrosis and cancer.

14. The use of a compound of claim 35, wherein the condition is prostatic fibrosis.

15. The use of a compound in claim 14, wherein the condition is selected from lower urinary tract symptoms and benign prostatic hyperplasia.

16-21. (canceled)

22. The compound of claim 1 a pharmaceutically acceptable salt, solvate, hydrate, polymorph, enantiomer, diastereomer, geometric isomer, racemate, tautomer, rotamer, atropisomer, isotopic variation, or N-oxide thereof, wherein
R3 is represented by Formula VIII: 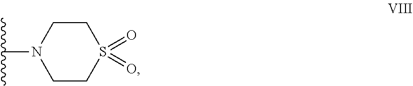 [SMILES: *N1CCS(=O)(=O)CC1]
A is a direct bond, and
B is NH.

23-26. (canceled)

27. The compound of claim 1, a pharmaceutically acceptable salt, solvate, hydrate, polymorph, enantiomer, diastereomer, geometric isomer, racemate, tautomer, rotamer, atropisomer, isotopic variation, or N-oxide thereof, wherein
R3 is represented by the Formula VIII: 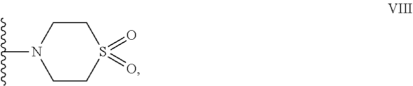 [SMILES: *N1CCS(=O)(=O)CC1]
A is a direct bond,
B is NH,
R1 is aryl or heteroaryl, and
R2 is alkyl, aryl or heteroaryl.

28. The compound of claim 1 a pharmaceutically acceptable salt, solvate, hydrate, polymorph, enantiomer, diastereomer, geometric isomer, racemate, tautomer, rotamer, atropisomer, isotopic variation, or N-oxide thereof, wherein
R3 is represented by Formula VIII: 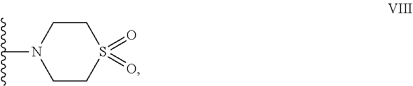 [SMILES: *N1CCS(=O)(=O)CC1]
A is a direct bond,
B is NH,
R1 is aryl or heteroaryl, and
R2 is alkyl, aryl or heteroaryl.

29. The compound of claim 1, a pharmaceutically acceptable salt, solvate, hydrate, polymorph, enantiomer, diastereomer, geometric isomer, racemate, tautomer, rotamer, atropisomer, isotopic variation, or N-oxide thereof, wherein
R3 is represented by Formula VIII: 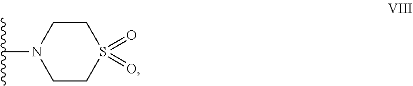 [SMILES: *N1CCS(=O)(=O)CC1]
A is a direct bond,
B is NH,
R2 is alkyl, aryl or heteroaryl,
R1 is -T-Q-(CH₂)ₙ—Z, wherein
T is aryl or heteroaryl
Q is C≡C.

30. The compound of claim 1 a pharmaceutically acceptable salt, solvate, hydrate, polymorph, enantiomer, diastereomer, geometric isomer, racemate, tautomer, rotamer, atropisomer, isotopic variation, or N-oxide thereof, wherein
R3 is represented by Formula VIII: 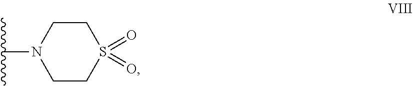 [SMILES: *N1CCS(=O)(=O)CC1]
A is a direct bond,
B is NH,
R2 is alkyl, aryl or heteroaryl,
R1 is -T-Q-(CH₂)ₙ—Z, wherein
T is aryl or heteroaryl, and
Q comprises C≡C.

31. The compound of claim 1, a pharmaceutically acceptable salt, solvate, hydrate, polymorph, enantiomer, diastereomer, geometric isomer, racemate, tautomer, rotamer, atropisomer, isotopic variation, or N-oxide thereof, selected from the group consisting of:
1-(4-chlorophenyl)-2-(2,4-dichlorophenyl)-N-(1,1-dioxidothiomorpholino)-5-methyl-1H-imidazole-4-carboxamide
ethyl 4-(2-(2,4-dichlorophenyl)-4-((1,1-dioxidothiomorpholino)carbamoyl)-5-methoxy-1H-imidazol-1-yl)benzoate
ethyl 4-(2-(4-chloro-2-fluorophenyl)-4-((1,1-dioxidothiomorpholino)carbamoyl)-5-methoxy-1H-imidazol-1-yl)benzoate
2-(2,4-dichlorophenyl)-N-(1,1-dioxidothiomorpholino)-1-(4-iodophenyl)-5-methyl-1H-imidazole-4-carboxamide
ethyl 4-(2-(2,4-dichlorophenyl)-4-((1,1-dioxidothiomorpholino)carbamoyl)-5-methyl-1H-imidazol-1-yl)benzoate
ethyl 4-(2-(2,4-dichlorophenyl)-4-((1,1-dioxidothiomorpholino)carbamoyl)-5-ethyl-1H-imidazol-1-yl)benzoate
ethyl 4-(2-(2,4-dichlorophenyl)-4-((1,1-dioxidothiomorpholino)carbamoyl)-5-(hydroxymethyl)-1H-imidazol-1-yl)benzoate
2-(2,4-dichlorophenyl)-N-(1,1-dioxidothiomorpholino)-1-(4-(4-isothiocyanatobut-1-yn-1-yl)phenyl)-5-methyl-1H-imidazole-4-carboxamide
4-(4-(2-(2,4-dichlorophenyl)-4-((1,1-dioxidothiomorpholino)carbamoyl)-5-methyl-1H-imidazol-1-yl)phenyl)but-3-yn-1-yl nitrate
2-(2,4-dichlorophenyl)-N-(1,1-dioxidothiomorpholino)-1-(4-(4-isothiocyanatobut-1-yn-1-yl)phenyl)-5-methoxy-1H-imidazole-4-carboxamide
1-(4-(4-cyanobut-1-yn-1-yl)phenyl)-2-(2,4-dichlorophenyl)-N-(1,1-dioxidothiomorpholino)-5-methyl-1H-imidazole-4-carboxamide
2-(4-chloro-2-fluorophenyl)-1-(4-(4-cyanobut-1-yn-1-yl)phenyl)-N-(1,1-dioxidothiomorpholino)-5-methyl-1H-imidazole-4-carboxamide
2-(4-chloro-2-fluorophenyl)-N-(1,1-dioxidothiomorpholino)-1-(4-(5-hydroxypent-1-yn-1-yl)phenyl)-5-methyl-1H-imidazole-4-carboxamide
2-(4-chloro-2-fluorophenyl)-N-(1,1-dioxidothiomorpholino)-1-(4-(5-hydroxypent-1-yn-1-yl)phenyl)-5-methoxy-1H-imidazole-4-carboxamide
Ethyl 6-(4-(2-(4-chloro-2-fluorophenyl)-4-((1,1-dioxidothiomorpholino)carbamoyl)-5-methoxy-1H-imidazol-1-yl)phenyl)hex-5-ynoate
2-(4-chloro-2-fluorophenyl)-1-(4-(5-cyanopent-1-yn-1-yl)phenyl)-N-(1,1-dioxidothiomorpholino)-5-methoxy-1H-imidazole-4-carboxamide
2-(2,4-dichlorophenyl)-N-(1,1-dioxidothiomorpholino)-1-(4-(4-hydroxybut-1-yn-1-yl)phenyl)-5-methyl-1H-imidazole-4-carboxamide
2-(2,4-dichlorophenyl)-N-(1,1-dioxidothiomorpholino)-1-(4-(4-hydroxybut-1-yn-1-yl)phenyl)-5-methoxy-1H-imidazole-4-carboxamide
1-(4-(4-cyanobut-1-yn-1-yl)phenyl)-2-(2,4-dichlorophenyl)-N-(1,1-dioxidothiomorpholino)-5-methoxy-1H-imidazole-4-carboxamide
4-(4-(2-(2,4-dichlorophenyl)-4-((1,1-dioxidothiomorpholino)carbamoyl)-5-methoxy-1H-imidazol-1-yl)phenyl)but-3-yn-1-yl nitrate
1-(4-(5-amino-5-oxopent-1-yn-1-yl)phenyl)-2-(2,4-dichlorophenyl)-N-(1,1-dioxidothiomorpholino)-5-methoxy-1H-imidazole-4-carboxamide
1-(4-(5-amino-5-oxopent-1-yn-1-yl)phenyl)-2-(2,4-dichlorophenyl)-N-(1,1-dioxidothiomorpholino)-5-methyl-1H-imidazole-4-carboxamide
1-(4-(4-cyanobut-1-yn-1-yl)phenyl)-2-(2,4-dichlorophenyl)-N-(1,1-dioxidothiomorpholino-2,2,3,3,5,5,6,6-d8)-5-methyl-1H-imidazole-4-carboxamide
2-(2,4-dichlorophenyl)-N-(1,1-dioxidothiomorpholino-2,2,3,3,5,5,6,6-d8)-1-(4-(4-hydroxybut-1-yn-1-yl)phenyl)-5-methyl-1H-imidazole-4-carboxamide
4-(4-(2-(2,4-dichlorophenyl)-4-((1,1-dioxidothiomorpholino-2,2,3,3,5,5,6,6-d8)carbamoyl)-5-methyl-1H-imidazol-1-yl)phenyl)but-3-yn-1-yl nitrate
4-(4-(2-(2,4-dichlorophenyl)-4-((1,1-dioxidothiomorpholino-2,2,3,3,5,5,6,6-d8)carbamoyl)-5-methoxy-1H-imidazol-1-yl)phenyl)but-3-yn-1-yl nitrate
1-(4-(4-cyanobut-1-yn-1-yl)phenyl)-2-(2,4-dichlorophenyl)-N-(1,1-dioxidothiomorpholino-2,2,3,3,5,5,6,6-d8)-5-methoxy-1H-imidazole-4-carboxamide
2-(2,4-dichlorophenyl)-N-(1,1-dioxidothiomorpholino-2,2,3,3,5,5,6,6-d8)-1-(4-(4-hydroxybut-1-yn-1-yl)phenyl)-5-methoxy-1H-imidazole-4-carboxamide
1-(4-(5-amino-5-oxopent-1-yn-1-yl)phenyl)-2-(2,4-dichlorophenyl)-N-(1,1-dioxidothiomorpholino-2,2,3,3,5,5,6,6-d8)-5-methoxy-1H-imidazole-4-carboxamide
1-(4-(5-amino-5-oxopent-1-yn-1-yl)phenyl)-2-(2,4-dichlorophenyl)-N-(1,1-dioxidothiomorpholino-2,2,3,3,5,5,6,6-d8)-5-methyl-1H-imidazole-4-carboxamide
1-(4-(4-cyanobut-1-yn-1-yl)phenyl)-2-(2,4-dichlorophenyl)-N-(1,1-dioxidothiomorpholino)-5-(methoxymethyl)-1H-imidazole-4-carboxamide
2-(2,4-dichlorophenyl)-N-(1,1-dioxidothiomorpholino)-1-(4-(4-hydroxybut-1-yn-1-yl)phenyl)-5-(methoxymethyl)-1H-imidazole-4-carboxamide
4-(4-(2-(2,4-dichlorophenyl)-4-((1,1-dioxidothiomorpholino)carbamoyl)-5-(methoxymethyl)-1H-imidazol-1-yl)phenyl)but-3-yn-1-yl nitrate
1-(4-(5-amino-5-oxopent-1-yn-1-yl)phenyl)-2-(2,4-dichlorophenyl)-N-(1,1-dioxidothiomorpholino)-5-(methoxymethyl)-1H-imidazole-4-carboxamide
1-(4-(4-cyanobut-1-yn-1-yl)phenyl)-2-(2,4-dichlorophenyl)-N-(1,1-dioxidothiomorpholino)-5-((methoxy-d3)methyl)-1H-imidazole-4-carboxamide
2-(2,4-dichlorophenyl)-N-(1,1-dioxidothiomorpholino)-1-(4-(4-hydroxybut-1-yn-1-yl)phenyl)-5-((methoxy-d3)methyl)-1H-imidazole-4-carboxamide
1-(4-(5-amino-5-oxopent-1-yn-1-yl)phenyl)-2-(2,4-dichlorophenyl)-N-(1,1-dioxidothiomorpholino)-5-((methoxy-d3)methyl)-1H-imidazole-4-carboxamide
4-(4-(2-(2,4-dichlorophenyl)-4-((1,1-dioxidothiomorpholino)carbamoyl)-5-((methoxy-d3)methyl)-1H-imidazol-1-yl)phenyl)but-3-yn-1-yl nitrate
3-((4-(2-(2,4-dichlorophenyl)-4-((1,1-dioxidothiomorpholino)carbamoyl)-5-methoxy-1H-imidazol-1-yl)phenyl)ethynyl)pyridine 1-oxide
3-((4-(2-(2,4-dichlorophenyl)-4-((1,1-dioxidothiomorpholino)carbamoyl)-5-methyl-1H-imidazol-1-yl)phenyl)ethynyl)pyridine 1-oxide
2-(2,4-dichlorophenyl)-N-(1,1-dioxidothiomorpholino)-5-methyl-1-(4-(pyridin-3-ylethynyl)phenyl)-1H-imidazole-4-carboxamide
2-(2,4-dichlorophenyl)-N-(1,1-dioxidothiomorpholino)-5-methoxy-1-(4-(pyridin-3-ylethynyl)phenyl)-1H-imidazole-4-carboxamide
2-(2,4-dichlorophenyl)-N-(1,1-dioxidothiomorpholino-2,2,3,3,5,5,6,6-d8)-5-methyl-1-(4-(pyridin-3-ylethynyl)phenyl)-1H-imidazole-4-carboxamide
2-(2,4-dichlorophenyl)-N-(1,1-dioxidothiomorpholino-2,2,3,3,5,5,6,6-d8)-5-methoxy-1-(4-(pyridin-3-ylethynyl)phenyl)-1H-imidazole-4-carboxamide
tert-butyl 5-(4-(2-(2,4-dichlorophenyl)-4-((1,1-dioxidothiomorpholino)carbamoyl)-5-methyl-1H-imidazol-1-yl)phenyl)pent-4-ynoate
ethyl 5-(4-(2-(2,4-dichlorophenyl)-4-((1,1-dioxidothiomorpholino)carbamoyl)-5-methyl-1H-imidazol-1-yl)phenyl)pent-4-ynoate
1-(4-(5-cyanopent-1-yn-1-yl)phenyl)-2-(2,4-dichlorophenyl)-N-(1,1-dioxidothiomorpholino)-5-methyl-1H-imidazole-4-carboxamide
5-(4-(2-(2,4-dichlorophenyl)-4-((1,1-dioxidothiomorpholino)carbamoyl)-5-methyl-1H-imidazol-1-yl)phenyl)pent-4-yn-1-yl nitrate.

32-34. (canceled)

35. Use of a compound of claim 1 to treat, prevent or reverse an inflammatory condition related to liver, lung, kidney or prostrate disease in an individual or animal in need of treatment.

36. Use of a compound of claim 1 to treat, prevent or reverse an fibrosis of the liver, lung, kidney or prostrate disease in an individual or animal in need of treatment.
